(19) United States
(12) Patent Application Publication
(10) Pub. No.: US 2026/0242420 A1
(43) Pub. Date: Aug. 20, 2026

(54) TNFR1-INHIBITORY CYCLIC PEPTIDE HAVING NON-NATURAL AMINO ACID STRUCTURE

(71) Applicant: Asahi Kasei Pharma Corporation, Tokyo (JP)

(72) Inventors: Koichiro ARAI, Tokyo (JP); Kazufumi OKAWA, Tokyo (JP); Arata SASO, Tokyo (JP); Toshihiko SONE, Tokyo (JP); Tatsuya TOMIYAMA, Kawasaki City (JP); Haruaki KURASAKI, Kawasaki City (JP); Naoki OKADA, Kawasaki City (JP); Ayumu MATSUDA, Kawasaki City (JP); Tomoko ASHIZAWA, Kawasaki City (JP); Yoshiaki MASUDA, Kawasaki City (JP); Yutaka KOBAYASHI, Kawasaki City (JP)

(73) Assignee: Asahi Kasei Pharma Corporation, Tokyo (JP)

(21) Appl. No.: 19/644,794

(22) Filed: Apr. 10, 2026

Related U.S. Application Data
(63) Continuation of application No. PCT/JP2025/040566, filed on Nov. 20, 2025.

(30) Foreign Application Priority Data
Nov. 29, 2024 (JP) 2024-208719

(51) Int. Cl. C07K 7/64; A61K 38/00; A61P 37/02

(52) U.S. Cl. CPC C07K 7/64; A61P 37/02; A61K 38/00

(57) ABSTRACT

Provided is a cyclic peptide having a non-natural amino acid structure which selectively binds to TNFR1 and has excellent TNFR1 inhibitory activity. The present invention provides a cyclic peptide represented by the following general formula (1):

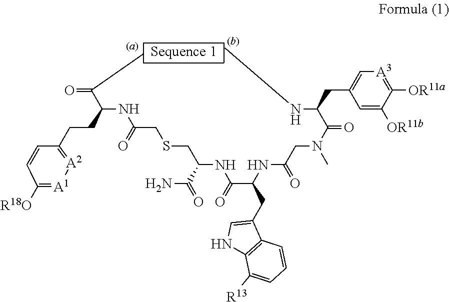

### BACKGROUND OF THE INVENTION

### Field of the Invention

The present invention relates to a cyclic peptide having a non-natural amino acid structure and having TNF receptor-1 (TNFR1) inhibitory activity.

### Description of the Related Art

TNFα (tumor necrosis factor α) was found as a factor inducing the necrosis of tumor cells and has been revealed to be a cytokine related to immunity or inflammation in subsequent research. TNFα not only has the action of directly causing inflammation by itself by simulating the skin or tissues, but also has the action of indirectly causing inflammation by activating macrophages or the like and thereby promoting the production of other inflammatory cytokines such as IL-1 (interleukin-1), IL-6 (interleukin-6), and IL-8 (interleukin-8). Overproduced TNFα in patients with autoimmune diseases is reported to be responsible for the development or aggravation of inflammation or tissue disorders.

Anti-TNFα antibodies, which are widely used as therapeutic drugs for autoimmune diseases, directly bind to TNFα and inhibit the binding of TNFα to a receptor on cell surface, thereby suppressing the production of inflammatory cytokines.

The receptors on cell surface to which TNFα binds include TNFR1 (TNF receptor-1) and TNER2 (TNF receptor-2). The difference between physiological activities of TNFα signals mediated by these receptors has become increasingly evident. For example, the possibility has been pointed out that the action of TNFα mediated by TNFR1 is involved in the initiation or aggravation of inflammatory reaction, while the action of TNFα mediated by TNER2 is involved in defense against viral infection or the suppression of pathological conditions of some diseases such as multiple sclerosis (see, for example, Masaki Inoue, et al., “Development of TNFR1-selective antagonistic TNF-α mutant as new biologics for treatment of immunological disorders”, Journal of Japanese Biochemical Society 93(3): 391-395 (2021)). The anti-TNFα antibodies inhibit both the signals of TNFα mediated by these receptors. Therefore, the anti-TNFα antibodies may suppress inflammatory reaction caused by the signal mediated by TNER1, and even the suppressive effect on defense against viral infection or the suppression of pathological conditions of multiple sclerosis ascribable to the signal mediated by TNER2 (see, for example, Vaisman-Mentesh et al., “The Molecular Mechanisms That Underlie the Immune Biology of Anti-drug Antibody Formation Following Treatment With Monoclonal Antibodies,” Front. Immunol., Volume 11 (2020)).

Accordingly, TNFR1 inhibitors which selectively bind to TNER1 of the TNFα receptors and selectively inhibit the action of TNFα mediated by TNFR1 are expected to have a therapeutic effect on autoimmune diseases (see, for example, Chinese Patent No. 102321155; Fischer et al., “Selective Targeting of TNF Receptors as a Novel Therapeutic Approach”, Front. Cell Dev. Biol., Volume 8, Article 401 (2020); Dong et al., “Targeting of Tumor Necrosis Factor Alpha Receptors as a Therapeutic Strategy for Neurodegenerative Disorders”, Antibodies, Volume 4, 369-408 (2015); and Zhang et al., “Selective inhibition of Tumor necrosis factor receptor-1 (TNFR1) for the treatment of autoimmune diseases”, Cytokine & Growth Factor Reviews, Volume 55, 80-85 (2020)). For example, there are reports on TNFα variants obtained by engineering the TNFR1 ligand TNFα so as to inhibit only a TNFR1 signal (see, for example, International Publication No. WO 2005/066206; Masaki Inoue, et al., “Development of TNFR1-selective antagonistic TNF-α mutant as new biologics for treatment of immunological disorders”, Journal of Japanese Biochemical Society 93(3): 391-395 (2021)), antibody variants that selectively inhibit TNFR1 (see, for example, International Publication No. WO 2022/047243), and cyclic peptides that selectively inhibit TNFR1 (see, for example, International Publication No. WO 2024/163691).

Although research has been made on cyclic peptides capable of binding to various molecules or receptors (see, for example, International Publication No. WO 2017/176608), some points have not yet been solved as to the relationship between the structures and functions or properties of the cyclic peptides in the present situation. In particular, attempts have been made to improve the functionality of cyclic peptides by using artificially synthesized non-natural amino acids (see, for example, U.S. Patent Application Publication No. 2023/0257343; International Publication No. WO 2021/090855; International Publication No. WO 2021/146441; Merz et al., “De novo development of small cyclic peptides that are orally bioavailable”, Nat Chem Biol 20, 624-633 (2024); and Tsukamoto et al., “In vitro display evolution of the PURE system-expressed TNFα-binding unnatural cyclic peptide containing an N-methyl-D-amino acid”, Biochemical and Biophysical Research Communications, Volume 534, 519-525 (2021)), but much remains unclear as to structures that improve the affinity for various targets, membrane permeability, solubility, and the like of cyclic peptides. There exist a plurality of non-natural amino acids that have not been introduced in cyclic peptides.

An object of the present invention is to provide a cyclic peptide having a non-natural amino acid structure which selectively binds to TNFR1 and has excellent TNFR1 inhibitory activity.

### SUMMARY OF THE INVENTION

The present inventors have conducted diligent studies to attain the object and consequently completed the present invention by finding that a cyclic peptide that is constituted by 14 amino acids and has a non-natural amino acid structure at a particular site has excellent TNFR1 selectivity and has excellent TNFR1 inhibitory activity.

Specifically, the present invention is as follows.
[1] A compound represented by the following general formula (1) or a salt thereof:

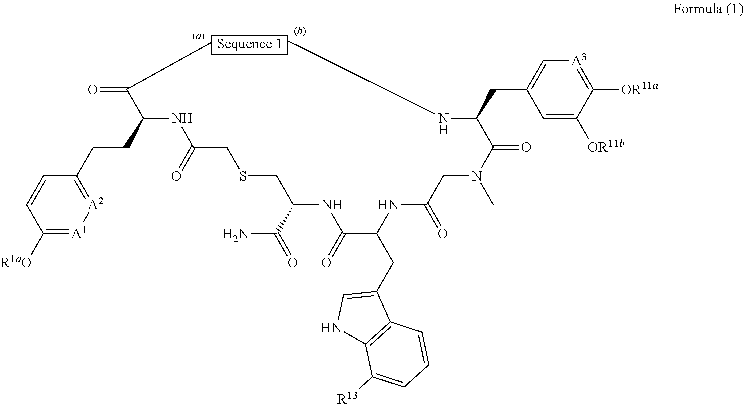

wherein
   the sequence 1 is an amino acid sequence represented by the following general formula (1-1):

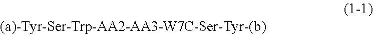

A¹ and A² are each independently CH or N;
R^{1a} is C₁₋₃ alkyl;
A³ is CH or N;
R^{11a} and R^{11b} are each independently C₁₋₃ alkyl;
R¹³ is 5- or 6-membered heteroaryl, and R¹³ is optionally substituted with one or two identical or different substituents selected from a G^{Ar} group;
the G^{Ar} group is the group consisting of —F, —Cl, cyano, C₁₋₃ alkyl, halogeno-C₁₋₃ alkyl, C₁₋₃ alkoxy-C₂₋₃ alkyl, —C(O)OH, and —C(O)NH₂;
AA1 is an amino acid structure represented by the following general formula (2-1):

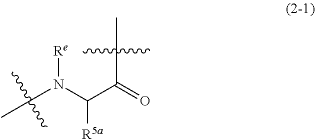

R^{e} is —H or C₁₋₃ alkyl, and C₁₋₃ alkyl of R^{e} is optionally substituted with —C(O)OH;
R^{5a} is —H, C₁₋₄ alkyl, halogeno-C₁₋₃ alkyl, or C₁₋₃ alkoxy-C₁₋₃ alkyl, and R^{5a} is optionally substituted with a substituent selected from a G¹ group;
the G¹ group is the group consisting of hydroxy, —NHR^{5b}, —C(O)OH, and —C(O)NH₂;
R^{5b} is a residue represented by the following general formula (2-1-1);

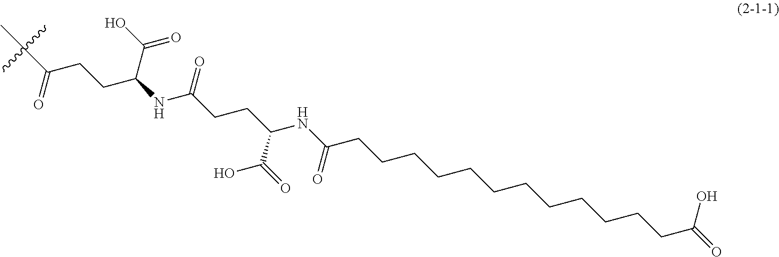

AA2 is an amino acid structure represented by the following general formula (2-2):

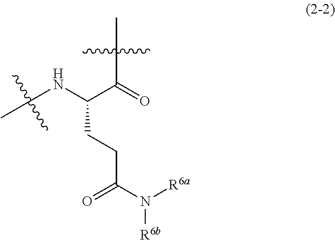

or an amino acid structure represented by the following general formula (2-3):

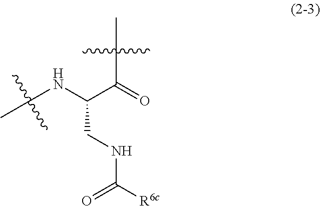

R^{6a} and R^{6b} are each independently —H, C₁₋₄ alkyl, halogeno-C₁₋₄ alkyl, hydroxy-C₁₋₄ alkyl, C₁₋₃ alkoxy-C₁₋₄ alkyl, or a 3- to 6-membered saturated ring group, and each of R^{6a} and R^{6b} is optionally substituted with a substituent selected from a G² group;
the G² group is the group consisting of —NH₂, 5- or 6-membered heteroaryl, and a 4- to 6-membered saturated ring group, and the 4- to 6-membered saturated ring group optionally has an N atom; or
R^{6a} and R^{6b} optionally form a 4- to 6-membered saturated ring together, and the 4- to 6-membered saturated ring is optionally substituted with C₁₋₃ alkyl-NH₂;
R^{6c} is C₁₋₃ alkyl;
AA3 is an amino acid structure represented by the following general formula (2-4):

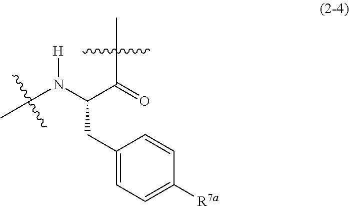

and
   R^{7a} is —H, hydroxy, or —C(O)OH.
[2] The compound or the salt thereof according to [1], wherein R^{1a} is methyl.
[3] The compound or the salt thereof according to [1] or [2], wherein A¹ is CH, A² is N, and R^{1a} is methyl.
[4] The compound or the salt thereof according to any of [1] to [3], wherein both of R^{11a} and R^{11b} are methyl.
[5] The compound or the salt thereof according to any of [1] to [4], wherein A³ is N, and both of R^{11a} and R^{11b} are methyl.
[6] The compound or the salt thereof according to any of [1] to [4], wherein A³ is CH, and both of R^{11a} and R^{11b} are methyl.
[7] The compound or the salt thereof according to any of [1] to [6], wherein the heteroatom of R¹³ is N.
[8] The compound or the salt thereof according to any of [1] to [7], wherein R¹³ has one or two heteroatoms.
[9] The compound or the salt thereof according to any of [1] to [8], wherein R¹³ is pyrrolyl, pyrazolyl, pyridyl, pyrazinyl, pyridazinyl, pyrimidinyl, or imidazolyl.
[10] The compound or the salt thereof according to any of [1] to [9], wherein R¹³ is pyrazolyl, pyridyl, pyridazinyl, or pyrimidinyl.
[11] The compound or the salt thereof according to any of [1] to [10], wherein the G^{Ar} group is the group consisting of —F, C₁₋₃ alkyl, —C(O)OH, and —C(O)NH₂.
[12] The compound or the salt thereof according to any of [1] to [11], wherein the G^{Ar} group is the group consisting of —F, methyl, —C(O)OH, and —C(O)NH₂.
[13] The compound or the salt thereof according to any of [1] to [12], wherein R¹³ is pyrimidinyl.
[14] The compound or the salt thereof according to any of [1] to [13], wherein R¹³ has the following structure:

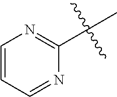 [SMILES: *c1ncccn1]

[15] The compound or the salt thereof according to any of [1] to [12], wherein R¹³ is pyridyl.
[16] The compound or the salt thereof according to any of [1] to [12], wherein R¹³ is pyridyl substituted with one —C(O)OH.
[17] The compound or the salt thereof according to any of [1] to [12], wherein R¹³ is pyridyl substituted with one —C(O)NH₂.
[18] The compound or the salt thereof according to any of [1] to [12], wherein R¹³ is pyridazinyl.
[19] The compound or the salt thereof according to any of [1] to [12], wherein R¹³ is pyrimidinyl substituted with one —F.
[20] The compound or the salt thereof according to any of [1] to [12], wherein R¹³ is pyrazolyl substituted with one methyl.
[21] The compound or the salt thereof according to any of [1] to [20], wherein R^{5a} is —H or C₁₋₃ alkyl, and the G¹ group is —C(O)OH.
[22] The compound or the salt thereof according to any of [1] to [21], wherein R^{e} is methyl, R^{5a} is methyl or ethyl, and the G¹ group is —C(O)OH.
[23] The compound or the salt thereof according to any of [1] to [22], wherein both of R^{e} and R^{5a} are methyl.
[24] The compound or the salt thereof according to any of [1] to [20], wherein R^{e} is ethyl substituted with —C(O)OH, and R^{5a} is —H.
[25] The compound or the salt thereof according to any of [1] to [22], wherein R^{e} is methyl, R^{5a} is butyl substituted with —NHR^{5b}, and R^{5b} is a residue represented by the general formula (2-1-1).
[26] The compound or the salt thereof according to any of [1] to [25], wherein AA2 is an amino acid represented by the general formula (2-2).
[27] The compound or the salt thereof according to any of [1] to [26], wherein each of R^{6a} and R^{6b} is —H.
[28] The compound or the salt thereof according to any of [1] to [26], wherein R^{6a} and R^{6b} form a 4-membered saturated ring together, and the 4-membered saturated ring is substituted with methyl —NH₂.
[29] The compound or the salt thereof according to any of [1] to [26], wherein one of R^{6a} and R^{6b} is —H, and the other is propyl substituted with —NH₂.
[30] The compound or the salt thereof according to any of [1] to [26], wherein one of R^{6a} and R^{6b} is —H, and the other is ethyl substituted with nitrogen-containing 6-membered heteroaryl.
[31] The compound or the salt thereof according to any of [1] to [26], wherein one of R^{6a} and R^{6b} is —H, and the other is ethyl substituted with —NH₂.
[32] The compound or the salt thereof according to any of [1] to [26], wherein one of R^{6a} and R^{6b} is —H, and the other is methyl substituted with a 5-membered saturated ring group having an N atom.
[33] The compound or the salt thereof according to any of [1] to [25], wherein AA2 is an amino acid represented by the general formula (2-3).
[34] The compound or the salt thereof according to any of [1] to [33], wherein Roc is methyl.
[35] The compound or the salt thereof according to any of [1] to [34], wherein R^{7a} is hydroxy.
[36] The compound or the salt thereof according to any of [1] to [34], wherein R^{1a} is —C(O)OH.
[37] The compound or the salt thereof according to [1], wherein A¹ is CH, A² is N, R^{1a} is methyl, A³ is N, both of R^{11a} and R^{11b} are methyl, R¹³ is pyrimidinyl, R^{e} is ethyl substituted with —C(O)OH, R^{5a} is —H, AA2 is an amino acid represented by the general formula (2-3), Roc is methyl, and R^{a} is hydroxy.
[38] A TNER1 inhibitor including the compound or the salt thereof according to any of [1] to [37].
[39] A pharmaceutical composition including the compound or the salt thereof according to any of [1] to as an active ingredient.
[40] The pharmaceutical composition according to for treatment of a disease related to the inhibition of TNFR1
[41] The pharmaceutical composition according to [40], wherein the disease is selected from the group consisting of ulcerative colitis, Crohn's disease, rheumatoid arthritis, psoriasis, ankylosing spondylitis, and hidradenitis suppurative.
[42] The pharmaceutical composition according to [40], wherein the disease is Crohn's disease.
[43] The pharmaceutical composition according to any of [39] to [42], wherein the pharmaceutical composition is subcutaneously administered or intravenously administered.
[44] The pharmaceutical composition according to any of to [42], wherein the pharmaceutical composition is subcutaneously administered.
[45] A compound represented by the following formula (3) or a salt thereof:

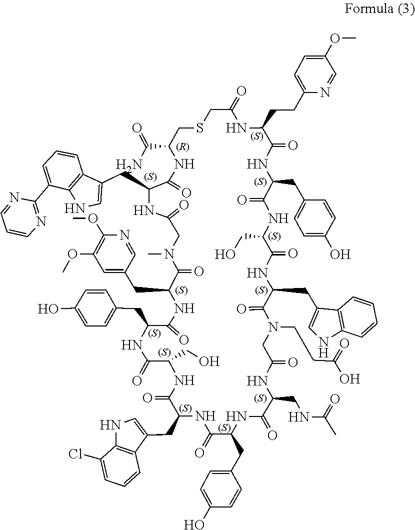

[46] A method for producing a compound or a salt thereof according to any of [1] to [37], the method including:
   providing 14 amino acids constituting the general formula (1), the amino acids being N-terminally protected with a 9-fluorenylmethyloxycarbonyl (Fmoc) group;
   repeating an operation of sequentially coupling the amino acids and removing the Fmoc group from the coupled amino acids to form a peptide; and
   cyclizing the peptide to obtain a compound represented by the general formula (1) or a salt thereof.
[47] A method for treating a disease related to the inhibition of TNFR1, the method including administering a compound or a salt thereof according to any of [1] to [37] to a subject.
[48] The compound or the salt thereof according to any of [1] to for use in the treatment of a disease related to the inhibition of TNFR1.
[49] Use of a compound or a salt thereof according to any of [1] to for production of a therapeutic agent for a disease related to the inhibition of TNER1.
[50] A cyclic peptide selected from:
(1) ClAc—H2PyOMe-Y—S—W-MeA-Qpyrli3RMe-F4COO—W7C—S—Y—F34dOMe-MeG-W72Py-C—NH2;
(2) ClAc—H2PyOMe-Y—S—W-MeA-QaEt-Y—W7C—S—Y-3Py56dOMe-MeG-W72pyrm-C—NH2;
(3) ClAc—H2PyOMe-Y—S—W-MeA-Qpyrli3RMe-F4COO—W7C—S—Y-3Py56dOMe-MeG-W72pyrm-C—NH2;
(4) ClAc—H2PyOMe-Y—S—W-MeA-QaPr—F4COO—W7C—S—Y-3Py56dOMe-MeG-W72pyrm-C—NH2;
(5) ClAc—H2PyOMe-Y—S—W-MeA-DapAc—Y—W7C—S—Y-3Py56dOMe-MeG-W72pyrm-C—NH2;
(6) ClAc—H2PyOMe-Y—S—W-MeA-Qpyrli3RMe-F4COO—W7C—S—Y-3Py56dOMe-MeG-W72Pyrm5F—C—NH2;
(7) ClAc—H2PyOMe-Y—S—W—CeG-DapAc—Y—W7C—S—Y-3Py56dOMe-MeG-W72pyrm-C—NH2;
(8) ClAc—H2PyOMe-Y—S—W-MeA-Q-Y—W7C—S—Y-3Py56dOMe-MeG-W72pyrm-C—NH2;
(9) ClAc—H2PyOMe-Y—S—W-MeA-Q-F4COO—W7C—S—Y-3Py56dOMe-MeG-W72pyrm-C—NH2;
(10) ClAc—H2PyOMe-Y—S—W—CeG-Q-Y—W7C—S—Y-3Py56dOMe-MeG-W72pyrm-C—NH2;
(11) ClAc—H2PyOMe-Y—S—W—CeG-Qpyrli3RMe-Y—W7C—S—Y-3Py56dOMe-MeG-W72pyrm-C—NH2;
(12) ClAc—H2PyOMe-Y—S—W-MeA-Q4Pye-F4COO—W7C—S—Y-3Py56dOMe-MeG-W72pyrm-C—NH2;
(13) ClAc—H2PyOMe-Y—S—W-MeD-DapAc—Y—W7C—S—Y-3Py56dOMe-MeG-W72pyrm-C—NH2;
(14) ClAc—H2PyOMe-Y—S—W-MeA-Qpyrli3RMe-F4COO—W7C—S—Y-3Py56dOMe-MeG-W72Py-C—NH2;
(15) ClAc—H2PyOMe-Y—S—W-MeA-QaMe3Az-F4COO—W7C—S—Y-3Py56dOMe-MeG-W72Py-C—NH2;
(16) ClAc—H2PyOMe-Y—S—W-MeA-QaMe3Az-Y—W7C—S—Y-3Py56dOMe-MeG-W73Pyrd-C—NH2;
(17) ClAc—H2PyOMe-Y—S—W-MeA-DapAc-F4COO—W7C—S—Y-3Py56dOMe-MeG-W73Pyrd-C—NH2;
(18) ClAc—H2PyOMe-Y—S—W-MeA-DapAc—Y—W7C—S—Y-3Py56dOMe-MeG-W73Pyrd-C—NH2;
(19) ClAc—H2PyOMe-Y—S—W-MeA-QaMe3Az-F4COO—W7C—S—Y—F34dOMe-MeG-W72Py5COO—C—NH2;
(20) ClAc—H2PyOMe-Y—S—W-MeA-QaMe3Az-F4COO—W7C—S—Y—F34dOMe-MeG-W72pyrm-C—NH2;
(21) ClAc—H2PyOMe-Y—S—W—CeG-QaMe3Az-F4COO—W7C—S—Y—F34dOMe-MeG-W72pyrm-C—NH2;
(22) ClAc—H3Pyrd6OMe-Y—S—W-MeA-QaMe3Az-F4COO—W7C—S—Y-3Py56dOMe-MeG-W72pyrm-C—NH2;
(23) ClAc—H2PyOMe-Y—S—W-MeE-QaPr—Y—W7C—S—Y-3Py56dOMe-MeG-W72pyrm-C—NH2;
(24) ClAc—H2PyOMe-Y—S—W-MeA-QaPr—F4COO—W7C—S—Y-3Py56dOMe-MeG-W73Pyz1Me-C—NH2;
(25) ClAc—H2PyOMe-Y—S—W-MeA-DapAc—Y—W7C—S—Y-3Py56dOMe-MeG-W72Py5COO—C—NH2;
(26) ClAc—H2PyOMe-Y—S—W-MeA-Qpyrli3RMe-Y—W7C—S—Y-3Py56dOMe-MeG-W72Py5CON—C—NH2;
(27) ClAc—H2PyOMe-Y—S—W-MeA-QaMe3Az-F4COO—W7C—S—Y—F34dOMe-MeG-W72Py5CON—C—NH2;
(28) ClAc—H2PyOMe-Y—S—W-MeA-QaMe3Az-F4COO—W7C—S—Y-3Py56dOMe-MeG-W73Pyrd-C—NH2;
(29) ClAc—H2PyOMe-Y—S—W-MeK (cC12COO-isoE-isoE-)-QaPr-F4COO—W7C—S—Y-3Py56dOMe-MeG-W72pyrm-C—NH2;
(30) ClAc—H2PyOMe-Y—S—W—CeG-DapAc—F4COO—W7C—S—Y-3Py56dOMe-MeG-W72pyrm-C—NH2; and
(31) ClAc—H2PyOMe-Y—S—W—CeG-Q-F4COO—W7C—S—Y-3Py56dOMe-MeG-W72pyrm-C—NH₂,
or a salt thereof.

The present invention can provide a cyclic peptide which selectively binds to TNFR1 and has excellent TNER1 inhibitory activity. The cyclic peptide can be used as a therapeutic agent for autoimmune diseases.

### BRIEF DESCRIPTION OF THE DRAWINGS

FIG. 1 is a graph showing the action of the compound of Example 7 on the amount of mouse IL-6 produced in plasma induced by human TNFα; and

FIG. 2 is a graph showing the action of the compound of Example 7 on enteritis in a TNF-ΔARE/human TNFR1 KI mouse.

### DETAILED DESCRIPTION OF THE INVENTION

Hereinafter, the mode for carrying out the present invention (hereinafter, also referred to as an “embodiment”) will be described. The present invention is not limited to embodiments given below and can be carried out through various changes or modifications made without departing from the spirit of the present invention. The embodiments given below illustrate methods and the like for embodying the technical belief of this invention and are not limited to these examples.

### [Compound of Formula (1)]

The compound according to an embodiment is a cyclic peptide represented by the following general formula (1):

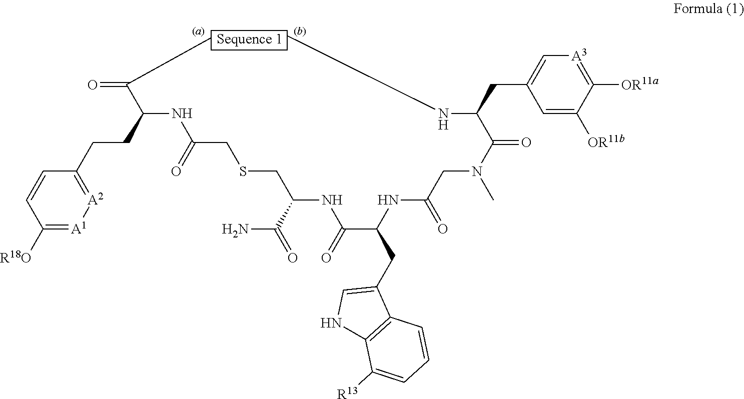

wherein
the sequence 1 is an amino acid sequence represented by the following general formula (1-1):

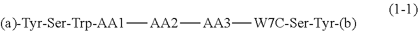

A¹ and A² are each independently CH or N;
R^{1a} is C₁₋₃ alkyl;
A³ is CH or N;
R^{11a} and R^{11b} are each independently C₁₋₃ alkyl;
R¹³ is 5- or 6-membered heteroaryl, and R¹³ is optionally substituted with one or two identical or different substituents selected from a G^{Ar} group;
the G^{Ar} group is the group consisting of —F, —Cl, cyano, C₁₋₃ alkyl, halogeno-C₁₋₃ alkyl, C₁₋₃ alkoxy-C₂₋₃ alkyl, —C(O)OH, and —C(O)NH₂;
AA1 is an amino acid structure represented by the following general formula (2-1):

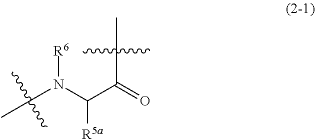

R^{e} is —H or C₁₋₃ alkyl, and C₁₋₃ alkyl of R^{e} is optionally substituted with —C(O)OH;
R^{5a} is —H, C₁₋₄ alkyl, halogeno-C₁₋₃ alkyl, or C₁₋₃ alkoxy-C₁₋₃ alkyl, and R^{5a} is optionally substituted with a substituent selected from a G¹ group;
the G¹ group is the group consisting of hydroxy, —NHR^{5b}, —C(O)OH, and —C(O)NH₂;
R^{5b} is a residue represented by the following general formula (2-1-1);

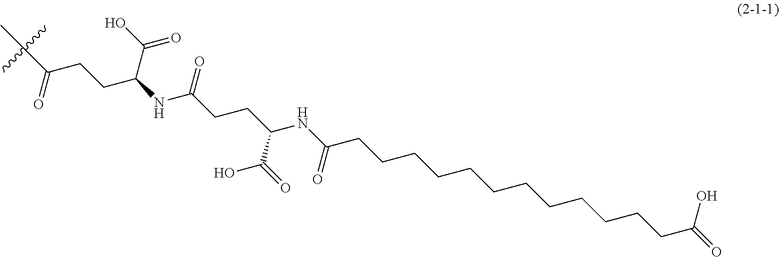

AA2 is an amino acid structure represented by the following general formula (2-2):

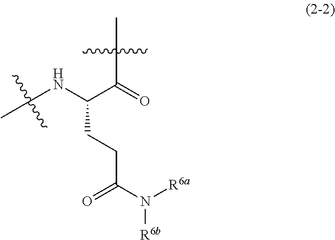

or an amino acid structure represented by the following general formula (2-3):

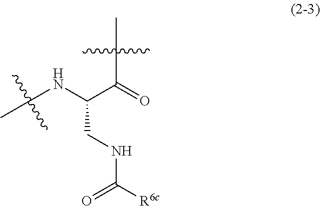

R^{6a} and R^{6b} are each independently —H, C₁₋₄ alkyl, halogeno-C₁₋₄ alkyl, hydroxy-C₁₋₄ alkyl, C₁₋₃ alkoxy-C₁₋₄ alkyl, or a 3- to 6-membered saturated ring group, and each of R^{6a} and R^{6b} is optionally substituted with a substituent selected from a G² group;
the G² group is the group consisting of —NH₂, 5- or 6-membered heteroaryl, and a 4- to 6-membered saturated ring group, and the 4- to 6-membered saturated ring group optionally has an N atom; or
R^{6a} and R^{6b} optionally form a 4- to 6-membered saturated ring together, and the 4- to 6-membered saturated ring is optionally substituted with C₁₋₃ alkyl-NH₂;
R^{6c} is C₁₋₃ alkyl;
AA3 is an amino acid structure represented by the following general formula (2-4):

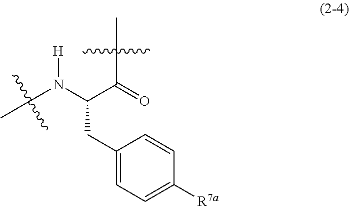

and
R^{7a} is —H, hydroxy, or —C(O)OH.

Absolute three-dimensional configuration has not been determined for most of the exemplary compounds in the present disclosure. However, the configuration is assigned thereto on the basis of similarity to particular compounds having a known chemical configuration (determined by X-ray crystallographic analysis) which are prepared using the same or similar reaction conditions and starting materials and isolated under the same chromatography conditions. Thus, the specific assignment of the configuration structurally expressed herein means that a specific compound prepared is confirmed to excessively have one particular stereoisomer, and is not necessarily construed herein as description about the absolute determination of a three-dimensional chemical structure of the compound unless otherwise specified in presented data.

“Halogeno” used in the present disclosure refers to a halogeno group consisting of a halogen atom. Examples of the “halogen atom” include atoms such as a fluorine atom (F), a chlorine atom (Cl), a bromine atom (Br), and an iodine (I) atom. The halogen atom is preferably a fluorine atom or a chlorine atom, more preferably a fluorine atom.

“C₁₋₃ alkyl” used in the present disclosure refers to a linear or branched alkyl group having one or more and three or less carbon atoms. Examples thereof include a methyl group, an ethyl group, a n-propyl group, and an iso-propyl group. The C₁₋₃ alkyl is preferably a methyl group, an ethyl group, or a n-propyl group, more preferably a methyl group or an ethyl group, further preferably a methyl group.

“C₁₋₄ alkyl” used in the present disclosure refers to a linear or branched alkyl group having one or more and four or less carbon atoms. Examples thereof include the specific examples of the “C₁₋₃ alkyl” described above as well as a n-butyl group, an iso-butyl group, a sec-butyl group, and a tert-butyl group.

In the present disclosure, the alkyl group having one carbon atom is also referred to as a “methyl group”, “methyl”, or “Me”.

In the present disclosure, the alkyl group having two carbon atoms is also referred to as an “ethyl group” or “ethyl”.

“Halogeno-C₁₋₃ alkyl” used in the present disclosure is a C₁₋₃ alkyl group substituted with at least one halogen atom, and “halogeno-C₁₋₄ alkyl” is a C₁₋₄ alkyl group substituted with at least one halogen atom. In this context, the “halogen atom”, the “C₁₋₃ alkyl”, and the “C₁₋₄ alkyl” are as described above. The at least one halogen atom may be the same or different. In the “halogeno-C₁₋₃ alkyl” and the “halogeno-C₁₋₄ alkyl”, the position of the halogen atom introduced in the C₁₋₃ alkyl group and the C₁₋₄ alkyl group is not limited.

“Hydroxy-C₁₋₄ alkyl” used in the present disclosure is a C₁₋₄ alkyl group substituted with at least one hydroxy group (—OH). In this context, the “C₁₋₄ alkyl” is as described above. In the “hydroxy-C₁₋₄ alkyl”, the position of the hydroxy group introduced in the C₁₋₄ alkyl group is not limited.

“C₁₋₃ alkyl-NH₂” used in the present disclosure is a C₁₋₃ alkyl group substituted with —NH₂. In this context, the “C₁₋₃ alkyl” is as described above. In the “C₁₋₃ alkyl-NH₂”, the position of —NH₂ introduced in the C₁₋₃ alkyl group is not limited.

“C₁₋₃ alkoxy” used in the present disclosure means an oxy group bonded to the “C₁₋₃ alkyl” described above. Examples thereof include a methoxy group, an ethoxy group, an n-propoxy group, and an iso-propoxy group. The C₁₋₃ alkoxy is preferably a methoxy group or an ethoxy group, more preferably a methoxy group.

“C₁₋₃ alkoxy-C₁₋₃ alkyl”, “C₁₋₃ alkoxy-C₂₋₃ alkyl”, and “C₁₋₃ alkoxy-C₁₋₄ alkyl” used in the present disclosure mean a C₁₋₃ alkyl group, a C₂₋₃ alkyl group, and a C₁₋₄ alkyl group, respectively, to which the “C₁₋₃ alkoxy” defined above is bonded. The “C₁₋₃ alkyl” and the “C₁₋₄ alkyl” are as described above. The “C₂₋₃ alkyl” refers to a linear or branched alkyl group having two or three carbon atoms. Examples thereof include an ethyl group, a n-propyl group, and an iso-propyl group.

“3- to 6-Membered saturated ring group” used in the present disclosure is a group having a saturated ring constituted by three to six atoms. All the atoms constituting the saturated ring may be carbon atoms, or one or more heteroatoms may be contained in the atoms constituting the saturated ring. The heteroatom is selected from the group consisting of a nitrogen atom (N), a sulfur atom(S) and an oxygen atom (O). Examples of the 3- to 6-membered saturated ring group include a cyclopropyl group, a cyclobutyl group, a cyclopentyl group, a cyclohexyl group, an aziridinyl group, an oxiranyl group, a thiiranyl group, an azetidinyl group, an oxetanyl group, a thietanyl group, a pyrrolidinyl group, a tetrahydrofuranyl group, a tetrahydrothienyl group, a pyrazolidinyl group, an imidazolidinyl group, an oxathiolanyl group, a piperidyl group, a piperazinyl group, a tetrahydropyranyl group, a morpholinyl group, a thiomorpholinyl group, a tetrahydro-2-pyron-yl group, a tetrahydrothiopyranyl group, a piperidin-2-on-yl group, a tetrahydrofuran-2-on-yl group, and a pyrrolidin-2-on-yl group.

“4- to 6-Membered saturated ring” used in the present disclosure is a saturated ring constituted by four to six atoms, and “4- to 6-membered saturated ring group” is a group having the 4- to 6-membered saturated ring. All the atoms constituting the saturated ring may be carbon atoms, or one or more heteroatoms may be contained in the atoms constituting the saturated ring. The heteroatom is selected from the group consisting of a nitrogen atom (N), a sulfur atom(S) and an oxygen atom (O). Examples of the 4- to 6-membered saturated ring group include a cyclobutyl group, a cyclopentyl group, a cyclohexyl group, an azetidinyl group, an oxetanyl group, a thietanyl group, a pyrrolidinyl group, a tetrahydrofuranyl group, a tetrahydrothienyl group, a pyrazolidinyl group, an imidazolidinyl group, an oxathiolanyl group, a piperidyl group, a piperazinyl group, a tetrahydropyranyl group, a morpholinyl group, a thiomorpholinyl group, a tetrahydro-2-pyron-yl group, a tetrahydrothiopyranyl group, a piperidin-2-on-yl group, a tetrahydrofuran-2-on-yl group, and a pyrrolidin-2-on-yl group.

“5- or 6-Membered heteroaryl” in the present disclosure means a monocyclic heteroaryl group that has five or six atoms constituting the ring of a cyclic group and contains one or more heteroatoms in the atoms constituting the ring of the cyclic group. The heteroatom is selected from the group consisting of a nitrogen atom (N), a sulfur atom(S) and an oxygen atom (O). Specific examples thereof include:
1) nitrogen-containing heteroaryl groups such as a pyrrolyl group, an imidazolyl group, a pyrazolyl group, a triazolyl group, a pyridyl group, a pyridazinyl group, a pyrimidinyl group, a pyrazinyl group, and a triazinyl group;
2) sulfur-containing heteroaryl groups such as a thienyl group;
3) oxygen-containing heteroaryl groups such as a furyl group; and
4) heteroaryl groups containing two or more different heteroatoms, such as a thiazolyl group, an oxazolyl group, an isothiazolyl group, and an isoxazolyl group. In this context, the “heteroaryl group” includes a pyridonyl group optionally having a substituent on a nitrogen atom. The “5- or 6-membered heteroaryl” in the present disclosure preferably contains one or two heteroatoms in the atoms constituting the ring of the cyclic group. The heteroatom is preferably a nitrogen atom (N), and the 5- or 6-membered heteroaryl is preferably a pyrrolyl group, a pyrazolyl group, a pyridyl group, a pyrazinyl group, a pyridazinyl group, or a pyrimidinyl group, more preferably a pyrazolyl group, a pyridyl group, a pyridazinyl group, or a pyrimidinyl group, further preferably a pyrimidinyl group.

“Hydroxy” or “hydroxy group” used in the present disclosure means a group represented by the formula —OH.

“Amino group” used in the present disclosure means a group represented by the formula —NH₂.

“Amide group” used in the present disclosure means a group represented by the formula —C(O)NH₂.

“Cyano” or “cyano group” used in the present disclosure means a group represented by the formula —CN.

“Carboxyl group” used in the present disclosure means a group represented by the formula —C(O)OH.

“Amino acid” in the present disclosure is a compound having a carboxyl group (—COOH) which is an acidic group and an amino group (—NH₂) which is a basic group in a molecule, and “amino acid structure” is a structure derived from each amino acid in a peptide composed of two or more amino acids linked through a peptide bond (—CONH—).

As shown in the formula (1), the compound according to an embodiment is a cyclic peptide obtained by cyclizing a peptide constituted by 14 amino acids. The peptide constituted by 14 amino acids is cyclized through a thioether structure. In the present disclosure, an amino acid structure having an unsaturated 6-membered ring substituted with OR^{1a} adjacent to the thioether structure in the formula (1) is defined as a first amino acid structure, and tyrosine (Tyr) at the (a) end of the sequence 1 adjacent to the first amino acid structure is defined as a second amino acid structure, followed by a third amino acid structure, a fourth amino acid structure, . . . a 13th amino acid structure, and a 14th amino acid structure in order. A structure derived from an amino acid having an indolyl group substituted with R¹³ is the 13th amino acid structure, and a structure derived from cysteine adjacent to the 13th amino acid structure, including the thioether structure, is the 14th amino acid structure. The first amino acid structure, the second amino acid structure, . . . the 13th amino acid structure, and the 14th amino acid structure are derived from a first amino acid, a second amino acid, . . . a 13th amino acid, and a 14th amino acid, respectively.

### (First Amino Acid Structure)

The first amino acid structure is a non-natural amino acid structure represented by the following general formula:

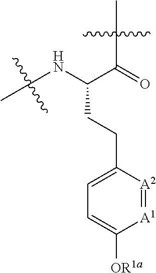

In the formula, A¹ and A² are each independently CH or N. In a preferred aspect, A² is N. In a preferred aspect, both of A¹ and A² may be N, or A¹ may be CH and A² may be N.

In the formula, R^{1a} is C₁₋₃ alkyl. Preferably, R^{1a} is C₁₋₃ alkyl, more preferably methyl or ethyl, further preferably methyl.

In one aspect, A² is N, and R^{1a} is methyl. In one aspect, the first amino acid structure has the following structure derived from H2PyOMe:

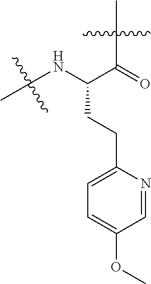 [SMILES: *N[C@@H](CCc1ccc(OC)cn1)C(*)=O]

or the following structure derived from H3Pyrd60Me:

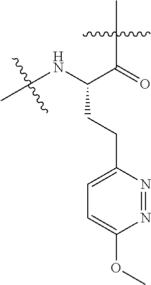 [SMILES: *N[C@@H](CCc1ccc(OC)nn1)C(*)=O]

### (Second Amino Acid Structure)

The second amino acid structure is the following structure derived from tyrosine (Tyr, Y):

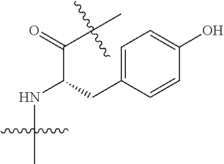 [SMILES: *N[C@@H](Cc1ccc(O)cc1)C(*)=O]

### (Third Amino Acid Structure)

The third amino acid structure is the following structure derived from serine (Ser, S):

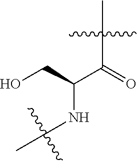 [SMILES: *N[C@@H](CO)C(*)=O]

### (Fourth Amino Acid Structure)

The fourth amino acid structure is the following structure derived from tryptophan (Trp, W):

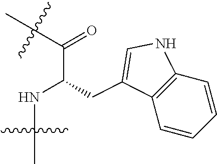 [SMILES: *N[C@@H](Cc1c[nH]c2ccccc12)C(*)=O]

### (Fifth Amino Acid Structure)

The fifth amino acid structure AA1 is an amino acid structure represented by the general formula (2-1) given below. The three-dimensional structure of the fifth amino acid structure is not limited and may be any of an L form and a D form. A bond between R^{5a} and a carbon atom represented by a straight line in the formula (2-1) may be a bond represented by a wedged line or a bond represented by a dashed line in dashed-wedged line drawing.

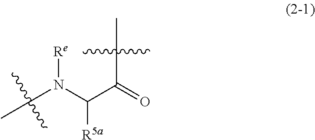

In the formula (2-1), R^{e} is —H or C₁₋₃ alkyl. R^{e} is preferably C₁₋₃ alkyl, further preferably methyl or ethyl. When R^{e} is C₁₋₃ alkyl, R^{e} is optionally substituted with —C(O)OH. The position of the substituent —C(O)OH in the C₁₋₃ alkyl is not limited.

In the formula (2-1), R^{5a} is —H, C₁₋₄ alkyl, halogeno-C₁₋₃ alkyl, or C₁₋₃ alkoxy-C₁₋₃ alkyl. R^{5a} is preferably —H or C₁₋₃ alkyl, further preferably —H, methyl, or ethyl.

In the formula (2-1), R^{5a} is optionally substituted with a substituent selected from a G¹ group. The G¹ group consists of hydroxy, —NHR^{5b}, —C(O)OH, and —C(O)NH₂. The position of the substituent of the G¹ group in R^{5a} is not limited. R^{5b} is a residue represented by the following general formula (2-1-1):

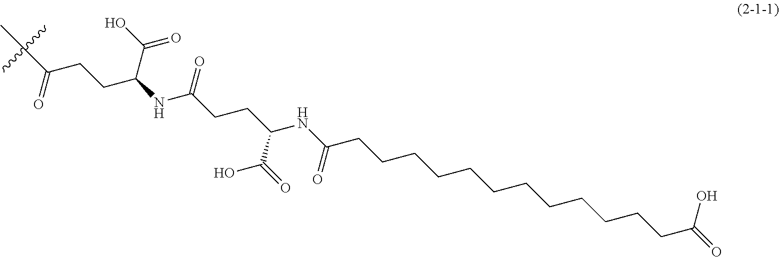

In one aspect, R^{e} is methyl or ethyl substituted with —C(O)OH, and R^{5a} is methyl or ethyl substituted with a substituent selected from the G¹ group.

In another aspect, R^{e} is —H or methyl or ethyl that is not substituted with —C(O)OH, and R^{5a} is methyl or ethyl that is not substituted with a substituent selected from the G¹ group. This aspect includes, for example, the following structure derived from MeA where both of R^{e} and R^{5a} are methyl:

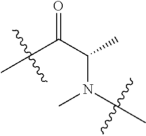 [SMILES: *C(=O)[C@H](C)N(*)C]

In a further alternative aspect, R^{e} is methyl or ethyl substituted with —C(O)OH, and R^{5a} is —H or methyl or ethyl that is not substituted with a substituent selected from the G¹ group. This aspect includes, for example, a structure derived from CeG where R^{e} is ethyl substituted with —C(O)OH, and R^{5a} is —H. Examples of the position of the substituent —C(O)OH in R^{e} include, but are not limited to, the following structure:

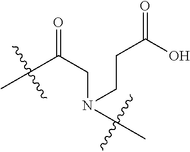 [SMILES: *C(=O)CN(*)CCC(=O)O]

In a further alternative aspect, R^{e} is —H or methyl or ethyl that is not substituted with —C(O)OH, and R^{5a} is methyl or ethyl substituted with a substituent selected from the G¹ group. The fifth amino acid structure according to this aspect includes, for example, a structure where R^{e} is —H, methyl, or ethyl, and R^{5a} is C₁₋₃ alkyl substituted with hydroxy, —NHR^{5b}, —C(O)OH, or —C(O)NH₂. Specifically, the fifth amino acid structure according to this aspect includes the following structures derived from MeD or MeE where R^{e} is methyl, and R^{5a} is methyl or ethyl substituted with —C(O)OH:

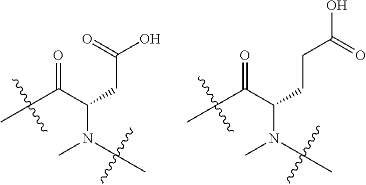 [SMILES: *C(=O)[C@H](CC(=O)O)N(*)C ; *C(=O)[C@H](CCC(=O)O)N(*)C]

and the following structure with a fatty acid derived from MeK where R^{e} is methyl, and R^{5a} is butyl substituted with —NHR^{5b}:

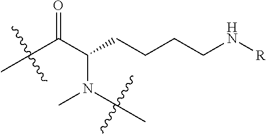

wherein R is

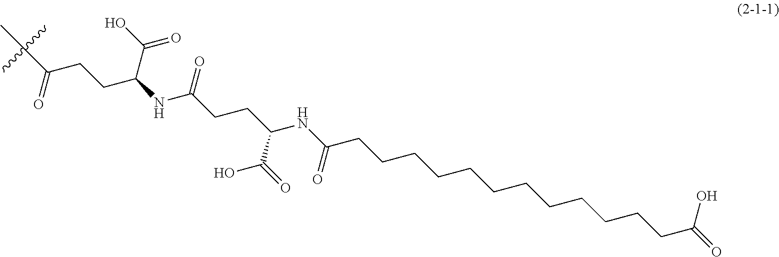 [SMILES: *C(=O)CC[C@H](NC(=O)CC[C@H](NC(=O)CCCCCCCCCCCCC(=O)O)C(=O)O)C(=O)O]

### (Sixth Amino Acid Structure)

The sixth amino acid structure AA2 is an amino acid structure represented by the following general formula (2-2) or the general formula (2-3):

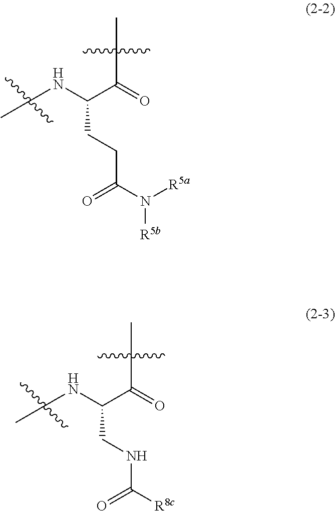

In the formula (2-2), R^{6a} and R^{6b} are each independently —H, C₁₋₄ alkyl, halogeno-C₁₋₄ alkyl, hydroxy-C₁₋₄ alkyl, C₁₋₃ alkoxy-C₁₋₄ alkyl, or a 3- to 6-membered saturated ring group.

Each of R^{6a} and R^{6b} is optionally substituted with a substituent selected from a G² group. The G² group is the group consisting of —NH₂, 5- or 6-membered heteroaryl, and a 4- to 6-membered saturated ring group. The 4- to 6-membered saturated ring group in the G² group optionally has an N atom. The position of the substituent of the G² group in R^{6a} and R^{6b} is not limited. The 4- to 6-membered saturated ring group having an N atom is, for example, an azetidinyl group, a pyrrolidinyl group, a pyrazolidinyl group, an imidazolidinyl group, a piperidyl group, or a piperazinyl group, preferably an azetidinyl group, a pyrrolidinyl group, or a piperidyl group, further preferably a pyrrolidinyl group.

In the formula (2-2), R^{6a} and R^{6b} optionally form a 4- to 6-membered saturated ring together. The 4- to 6-membered saturated ring is optionally substituted with C₁₋₃ alkyl-NH₂. The position of the substituent C₁₋₃ alkyl-NH₂ in the 4- to 6-membered saturated ring is not limited.

In the formula (2-3), Roc is C₁₋₃ alkyl. R^{6c} is preferably methyl or ethyl, further preferably methyl.

The structure of AA2 is preferably as follows:

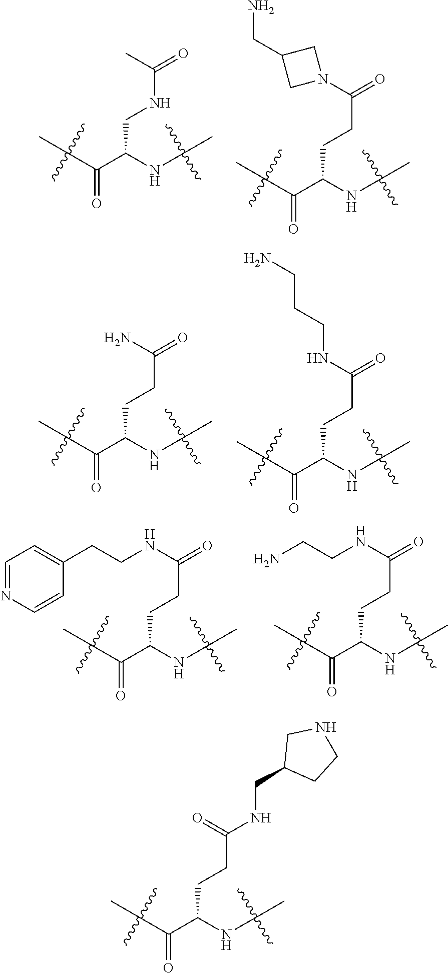

### (Seventh Amino Acid Structure)

The seventh amino acid structure AA3 is an amino acid structure represented by the following general formula (2-4).

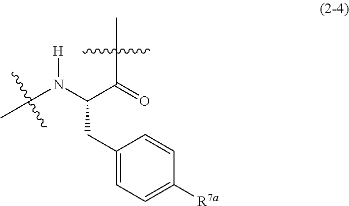

In the formula (2-4), R^{7a} is —H, hydroxy, or —C(O)OH. R^{7a} is preferably hydroxy or —C(O)OH.

The structure of AA3 is preferably as follows:

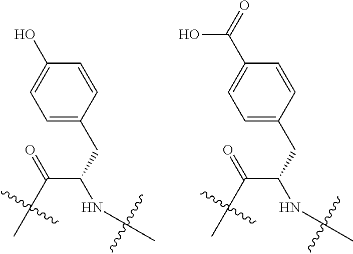 [SMILES: *N[C@@H](Cc1ccc(O)cc1)C(*)=O ; *N[C@@H](Cc1ccc(C(=O)O)cc1)C(*)=O]

### (Eighth Amino Acid Structure)

The eighth amino acid structure is the following structure derived from 7-chlorotryptophan (W7C):

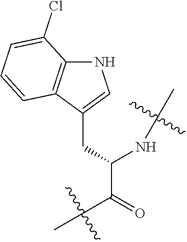 [SMILES: *N[C@@H](Cc1c[nH]c2c(Cl)cccc12)C(*)=O]

### (Nineth Amino Acid Structure)

The nineth amino acid structure is the following structure derived from serine (Ser, S):

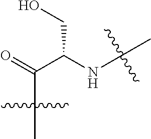 [SMILES: *N[C@@H](CO)C(*)=O]

### (10th Amino Acid Structure)

The 10th amino acid structure is the following structure derived from tyrosine (Tyr, Y):

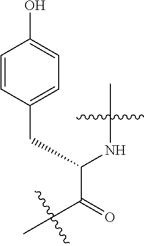 [SMILES: *N[C@@H](Cc1ccc(O)cc1)C(*)=O]

### (11th Amino Acid Structure)

The 11th amino acid structure is a non-natural amino acid structure represented by the following general formula:

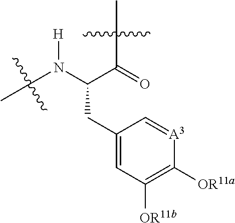

In the formula, A³ is CH or N.

In the formula, R^{11a} and R^{11b} are each independently C₁₋₃ alkyl, preferably methyl or ethyl, further preferably methyl. R^{11a} and R^{11b} may be the same or different. Preferably, R^{11a} and R^{11b} are the same, and both of them are, for example, methyl or ethyl. Further preferably, both of R^{11a} and R^{11b} are methyl. Particularly preferably, A³ is N, and both of R^{11a} and R^{11b} are methyl.

The structure of the 11th amino acid structure is preferably as follows:

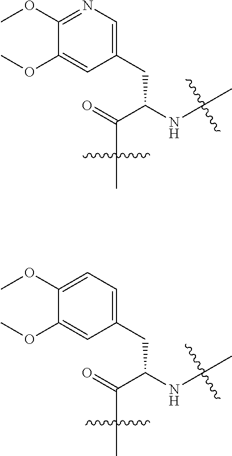 [SMILES: *N[C@@H](Cc1cnc(OC)c(OC)c1)C(*)=O ; *N[C@@H](Cc1ccc(OC)c(OC)c1)C(*)=O]

### (12th Amino Acid Structure)

The 12th amino acid structure is the following structure derived from methylglycine (MeG) (sarcosine):

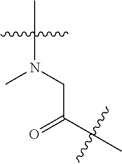 [SMILES: *C(=O)CN(*)C]

### (13th Amino Acid Structure)

The 13th amino acid structure is a non-natural amino acid structure represented by the following general formula:

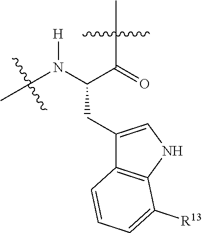

In the formula, R¹³ is 5- or 6-membered heteroaryl. The 5- or 6-membered heteroaryl is preferably nitrogen-containing heteroaryl having a nitrogen atom (N) as a heteroatom. The 5- or 6-membered heteroaryl having a nitrogen atom as a heteroatom includes, for example, a pyrrolyl group, an imidazolyl group, a pyrazolyl group, a pyridyl group, a pyrazinyl group, a pyridazinyl group, and a pyrimidinyl group and preferably includes a pyrazolyl group, a pyridyl group, a pyrazinyl group, a pyridazinyl group, and a pyrimidinyl group. In this context, the pyrazolyl group may be any of 1-pyrazolyl, 3-pyrazolyl, and 4-pyrazolyl and is preferably 3-pyrazolyl. The pyridyl group may be any of 2-pyridyl, 3-pyridyl, and 4-pyridyl and is preferably 2-pyridyl. The pyrazinyl group is preferably 2-pyrazinyl. The pyridazinyl group may be any of 3-pyridazinyl and 4-pyridazinyl and is preferably 3-pyridazinyl. The pyrimidinyl group may be any of 2-pyrimidinyl and 4-pyrimidinyl and preferably has a structure represented by

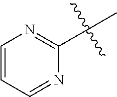 [SMILES: *c1ncccn1]

In the formula of the 13th amino acid structure, R¹³ is optionally substituted with one or two identical or different substituents selected from a G^{Ar} group. The G^{Ar} group is the group consisting of —F, —Cl, cyano, C₁₋₃ alkyl, halogeno-C₁₋₃ alkyl, C₁₋₃ alkoxy-C₂₋₃ alkyl, —C(O)OH, and —C(O)NH₂. The substituents included in the G^{Ar} group are preferably —F, C₁₋₃ alkyl, —C(O)OH, and —C(O)NH₂, further preferably —F, methyl, —C(O)OH, and —C(O)NH₂.

The position of the substituent of the G^{Ar} group in R¹³ is not limited. In one aspect, for example, when R¹³ is a 3-pyrazolyl group, the 1-position of the pyrazole ring may be substituted. When R¹³ is a 2-pyridyl group, the 5-position of the pyridine ring may be substituted. When R¹³ is a 2-pyrimidinyl group, the 5-position of the pyrimidine ring may be substituted.

The 13th amino acid structure is preferably a structure selected from the following structures:

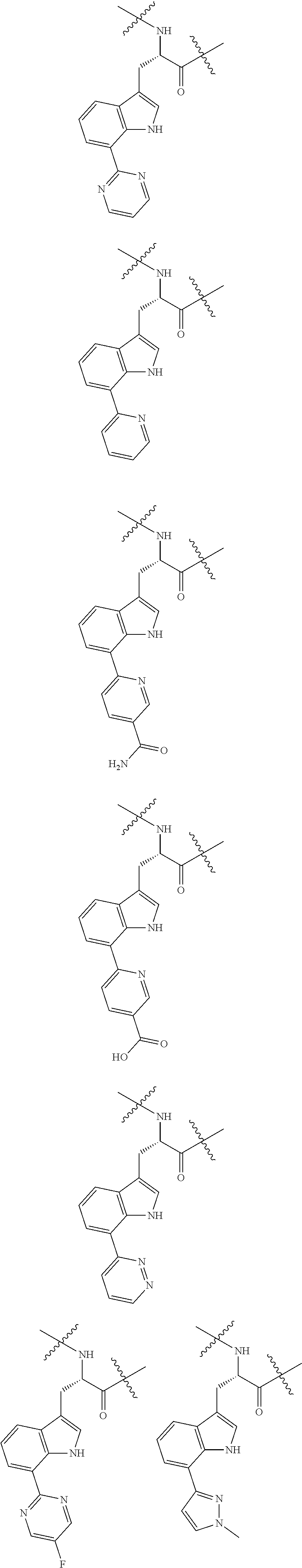

The 13th amino acid structure is particularly preferably the following structure:

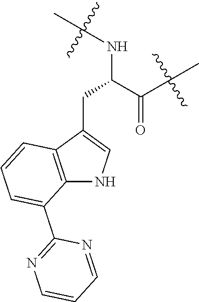 [SMILES: *N[C@@H](Cc1c[nH]c2c(-c3ncccn3)cccc12)C(*)=O]

### (14th Amino Acid Structure)

The 14th amino acid structure is an amino acid structure represented by the following formula:

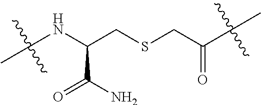 [SMILES: *N[C@@H](CSCC(*)=O)C(N)=O]

The first amino acid structure, the fifth amino acid structure (AA1), the sixth amino acid structure (AA2), the seventh amino acid structure (AA3), the 11th amino acid structure, and the 13th amino acid structure described above can be appropriately selected as proper structures from their respective aspects to form a cyclic peptide of the formula (1). A combination of the first amino acid structure to the 14th amino acid structure is not particularly limited and is preferably the following aspect:
a combination of the first amino acid structure to the 14th amino acid structure, wherein
in the first amino acid structure, A¹ is CH, A² is N, and R^{1a} is methyl;
in the fifth amino acid structure (AA1), R^{e} is ethyl substituted with —C(O)OH, and R^{5a} is —H;
in the sixth amino acid structure (AA2), AA2 is an amino acid represented by the general formula (2-3), and R^{6c} is methyl;
in the seventh amino acid structure (AA3), R^{7a} is hydroxy;
in the 11th amino acid structure, A³ is N, and both of R^{11a} and R^{11b} are methyl; and
in the 13th amino acid structure, R¹³ is a pyrimidinyl group having the following structure:

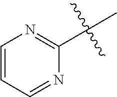 [SMILES: *c1ncccn1]

### [Salt]

The compound of the formula (1) described above may be in the form of a salt. The “salt” in the present disclosure is not particularly limited as long as the salt is formed with the compound according to an embodiment and is pharmacologically acceptable. Examples thereof include hydrohalide (e.g., hydrochloride, hydrobromide, and hydroiodide), inorganic acid salts (e.g., sulfate, nitrate, perchlorate, phosphate, carbonate, and bicarbonate), organic carboxylate (e.g., acetate, trifluoroacetate, maleate, tartrate, fumarate, and citrate), organic sulfonate (e.g., methanesulfonate, ethanesulfonate, benzenesulfonate, toluenesulfonate, and camphor sulfonate), amino acid salts (e.g., aspartate and glutamate), quaternary ammonium salts, alkali metal salts (e.g., sodium salt and potassium salt), and alkaline earth metal salts (magnesium salt and calcium salt). The salt is preferably hydrochloride, sulfate, methanesulfonate, acetate, or the like.

### [Method for Producing Compound of Formula (1) or Salt Thereof]

The cyclic peptide of the formula (1) described above may be chemically synthesized, may be recombinantly synthesized in a cell-free system, may be recombinantly synthesized in cells, or may be isolated from a biological source. The cyclic peptide of the formula (1) described above can be produced by a method known in the art such as these methods. The cyclic peptide can be chemically synthesized by use of diverse methods known in the art including, for example, stepwise solid-phase synthesis, liquid-phase synthesis, semisynthesis of peptide fragments through conformationally supported re-ligation, cloning, and enzymatic ligation or chemical ligation of synthetic peptide segments. For example, the synthesis method of liquid-phase synthesis is chemical synthesis as described in Anamika, S. et al., eds., CHEMICAL REVIEWS, p. 13516-13546 (2022). The method for synthesizing the cyclic peptide described herein and analogs thereof is preferably chemical synthesis using various solid-phase techniques as described in Chan, W. C. et al., eds., Fmoc Solid Phase Synthesis (572) JP 6387082 B2 2018.9.5, Oxford University Press, Oxford (2000); Barany, G. et al., The Peptides: Analysis, Synthesis, Biology, Vol. 2: “Special Methods in Peptide Synthesis, Part A”, pp. 3-284, Gross, E. et al., eds., Academic Press, New York (1980); and Stewart, J. M. et al., Solid-Phase Peptide Synthesis, 2nd Edition, Pierce Chemical Co., Rockford, IL (1984). A preferred synthesis strategy is a combination of a Fmoc (9-fluorenylmethylmethyl-oxycarbonyl) group for transient protection of an α-amino group and a tert-butyl group for transient protection of an amino acid side chain (e.g., Therton, E. et al., “The Fluorenylmethoxycarbonyl Amino Protecting Group”, in The Peptides: Analysis, Synthesis, Biology, Vol. 9: “Special Methods in Peptide Synthesis, Part C”, pp. 1-38, Undenfriend, S. et al., eds., Academic Press, San Diego (1987)).

In general, a peptide can be synthesized, starting at the C terminus of the peptide, by a stepwise method on an insoluble polymer support (also called “resin”). The synthesis is initiated by the binding of the C-terminal amino acid of the peptide to the resin through the formation of an amide or ester bond. This permits liberation at a final stage as C-terminal amide or carboxylic acid of the obtained peptide.

The C-terminal amino acid and all other amino acids for use in synthesis need to have differently protected α-amino groups and side chain functionality (if present) so as to selectively remove α-amino protective groups during synthesis. Amino acid coupling is carried out by the activation of carboxyl groups thereof as active ester and the reaction of an N-terminal amino acid bound to the resin with a free α-amino group. The order of α-amino group deprotection and coupling is continued until a complete peptide sequence is formed. Subsequently, the peptide is usually liberated from the resin in the presence of an appropriate scavenger in order to control side reaction, while side chain functionality is deprotected. The obtained peptide is finally purified by reverse-phase HPLC.

A peptidyl-resin as a precursor of the final peptide can be synthesized using a commercially available cross-linked polystyrene polymer resin (Novabiochem, San Diego, CA; Applied Biosystems, Foster City, CA). The solid-phase support is preferably 4-(2′,4′-dimethoxyphenyl-Fmoc-aminomethyl)-phenoxyacetyl-p-methylbenzhydrylamineresin (Rink amide-MBHA resin) for C-terminal carboxamide; 9-Fmoc-amino-xanthen-3-yloxy-Merrifield resin (Sieber amide resin); or 4-(9-Fmoc)aminomethyl-3,5-dimethoxyphenoxy) valeryl-aminomethyl-Merrifield resin (PAL resin). The coupling of the first and subsequent amino acids can be achieved using HOBt or using 6-Cl-HOBt, Oxyma, or HOAt active ester produced from DIC/HOBt, HBTU/HOBt, BOP, PyBOP, DIC/6-Cl-HOBt, HCTU, DIC/HOAt, DIC/Oxyma pure, or HATU. The solid-phase support is preferably 9-Fmoc-amino-xanthen-3-yloxy-Merrifield resin (Sieber amide resin) of a protected peptide fragment. The loading of the Sieber amide resin with the first amino acid is optimally achieved by reacting the resin having the deprotected Fmoc group and a Fmoc-protected amino acid with a condensing agent that forms the active ester in DMF. If necessary, DIEA may be added thereto, and DCM or NMP may be used for promoting the dissolution of amino acids.

The synthesis of peptide analogs described herein can be carried out using a single- or multi-channel peptide synthesizer, for example, Liberty PRIME Microwave synthesizer or Liberty Blue Microwave synthesizer from CEM Corp., or Syro I or Syro II from Biotage Japan Ltd.

Thus, the cyclic peptide of the formula (1) described above can be synthesized, for example, as follows.

First, the 14 amino acids constituting the formula (1) are transiently protected at their N termini with a protective group. For example, an Fmoc group can be used as the protective group. The side chains of the amino acids may be transiently protected. For example, a monomethoxytrityl group, a trityl group, a tert-butyl group, an allyl group, a 3-(3-methyl) pentyl group, a 9-xanthenyl group, a tert-butoxycarbonyl group, and an Fmoc group can be used for the transient protection of the amino acid side chains. The first amino acid may be chloroacetylated. The chloroacetyl group may be introduced to the first amino acid before peptide synthesis or after synthesis of a peptide chain.

Next, a peptide is elongated by repeating an operation of sequentially coupling the N-terminally protected amino acids in order from the 14th amino acid to the first amino acid and removing the N-terminal protective group from the coupled amino acids. The peptide during elongation may be supported on a solid phase. The elongation of the peptide can be performed by automated synthesis. For example, Liberty PRIME or Liberty Blue from CEM Corp., or Syro I or Syro II from Biotage Japan Ltd. can be used as a solid-phase synthesizer in the automated synthesis. The N-terminal protective group can be removed, for example, by washing the resin on which the peptide is supported with N, N-dimethylformamide (DMF) or the like.

The peptide chain composed of the 14th amino acid to the first amino acid bound in order is cyclized by connecting the first amino acid structure and the 14th amino acid structure in the peptide chain to obtain a compound represented by the formula (1) or a salt thereof. The peptide cyclization can be performed, for example, by reacting the chloroacetyl group added to the first amino acid structure with a structure derived from cysteine of the 14th amino acid structure to form a thioether bond.

### [TNFR1 Inhibitor]

The compound of the formula (1) described above has TNFR1 inhibitory activity and can be used as a TNFR1 inhibitor. Thus, the compound of the formula (1) can be used as a medicament for the prevention and/or treatment of a disease related to the inhibition of TNFR1. The disease related to the inhibition of INFR1 is a disease that can be suppressed by the inhibition of TNFR1 and is not particularly limited as long as, more specifically, the disease can be prevented and/or treated by suppressing the production of an inflammatory mediator such as TNFα or IL-8 by the inhibition of the TNFα signal transduction system.

The TNFR1 inhibitory activity of the compound of the formula (1) can be measured, for example, by a method shown in Test Example 1 or Test Example 2 mentioned later.

The disease related to the inhibition of TNFR1 is not particularly limited as long as the disease can be suppressed by the inhibition of TNFR1. The disease is, for example, ulcerative colitis, Crohn's disease, rheumatoid arthritis, psoriasis, ankylosing spondylitis, or hidradenitis suppurative, further preferably Crohn's disease.

### [Pharmaceutical Composition]

An embodiment of the present invention may be a pharmaceutical composition including the compound of the formula (1) described above. The pharmaceutical composition preferably includes the compound of the formula (1) as an active ingredient.

The compound of the formula (1) or the pharmaceutical composition including the same is excellent in safety (various toxicities and safety pharmacology), pharmacokinetic performance, and the like. The usefulness of the compound as an active ingredient in the pharmaceutical composition can be confirmed, for example, by the following methods.

Examples of the test related to safety include, but are not limited to, cytotoxicity tests (tests using HL60 cells or hepatocytes, etc.), genotoxicity tests (Ames test, mouse lymphoma TK test, chromosomal aberration test, micronucleus test, etc.), skin sensitization tests (Buehler method, GPMT method, APT method, LLNA test, etc.), skin photosensitization tests (adjuvant and strip method, etc.), local irritation tests (single subcutaneous dose, repetitive subcutaneous doses, etc.), hemolysis tests, safety pharmacology tests for the cardiovascular system (telemetry method, APD method, hERG inhibition assay, etc.), safety pharmacology tests for the central nervous system (FOB method, modified version of Irwin method, etc.), safety pharmacology tests for the respiratory system (measurement method using a respiratory function measurement apparatus, measurement method using a blood gas analyzer, etc.), general toxicity tests, and reproductive and developmental toxicity tests.

Examples of the test related to pharmacokinetic performance include, but are not limited to, cytochrome P450 enzyme inhibition or induction tests, cell permeability tests (parallel artificial membrane permeability assay (PAMPA), tests using Caco-2 cells or MDCK cells, etc.), evaluation tests on interaction involving drug transporters, blood concentration transition measurement tests, metabolism tests (stability test, metabolite molecular species test, reactive metabolite production confirmation test, etc.), solubility tests (solubility test based on turbidity method, etc.), and tissue distribution tests (test to evaluate potential transfer into tissues including potential drug transfer into intestinal tissues).

The compound of the formula (1) according to a certain aspect can be confirmed to be safe as an active ingredient in the pharmaceutical composition by conducting, for example, a cytotoxicity test. Examples of the cytotoxicity test include methods using various cultured cells, for example, HL-60 cells which are human preleukemia cells, primary isolated cultured cells of hepatocytes, or neutrophil fractions prepared from human peripheral blood. This test can be carried out by the method described below, though the method is not limited only by the following description. Cells are prepared as a suspension of 10⁵ to 10⁷ cells/ml, and 0.01 to 1 mL of the suspension is added to a microtube, a microplate, or the like. A solution of the compound dissolved therein is added thereto in 1/100 to 1 times the amount of the cell suspension, and the cells are cultured at 37° C. under 5% CO₂ for 30 minutes to several days in a cell culture solution that attains, for example, 0.001 UM to 1000 UM as the final concentration of the compound. After the completion of culture, the survival rate of the cells is evaluated by use of the MTT method, the WST-1 method (Ishiyama, M., et al., In Vitro Toxicology, 8, p. 187, 1995), or the like. The safety as an active ingredient in the pharmaceutical composition can be confirmed by measuring the cytotoxicity of the compound to cells.

The compound of the formula (1) according to a certain aspect can be confirmed to be safe as an active ingredient in the pharmaceutical composition by conducting, for example, a genotoxicity test. Examples of the genotoxicity test include Ames test, mouse lymphoma TK test, chromosomal aberration test, and micronucleus test. The Ames test is a method of determining reverse mutation by using Salmonella or Escherichia bacteria of designated species and culturing the bacteria on a culture dish or the like supplemented with the compound (see IYAKUSHIN (Notification by the chief of Evaluation and Licensing Division, Pharmaceutical and Medical Safety Bureau, Ministry of Health, Labor and Welfare, Japan), No. 1604, 1999, “Guideline for Genotoxicity Test”, II-1. Genotoxicity Test, etc.). The mouse lymphoma TK test is a genetic mutagenic potential detection test targeting the thymidine kinase gene of mouse lymphoma L5178Y cells (see IYAKUSHIN No. 1604, 1999, “Guideline for Genotoxicity Test”, II-3. Mouse Lymphoma TK Test; Clive, D. et al., Mutat. Res., 31, pp. 17-29, 1975; Cole, J., et al., Mutat. Res., 111, pp. 371-386, 1983, etc.). The chromosomal aberration test is a method of determining activity of causing chromosomal aberration by culturing mammalian cultured cells in the presence of the compound, then fixing the cells, and staining and observing the chromosomes of the cells (refer to IYAKUSHIN No. 1604, 1999, “Guideline for Genotoxicity Test”, II-2. Chromosomal Aberration Test Using Mammalian Cultured Cells, etc.). The micronucleus test is a method of evaluating micronucleus forming ability caused by chromosomal aberration, the method using a rodent (in vivo test) (IYAKUSHIN No. 1604, 1999, “Guideline for Genotoxicity Test”, II-4. Micronucleus Test Using Rodent; Hayashi, M. et al., Mutat. Res., 312, pp. 293-304, 1994; and Hayashi, M. et al., Environ. Mol. Mutagen., 35, pp. 234-252, 2000) or the method using cultured cells (in vitro test) (Fenech, M. et al., Mutat. Res., 147, pp. 29-36, 1985; and Miller, B., et al., Mutat. Res., 392, pp. 45-59, 1997). The safety as an active ingredient in the pharmaceutical composition can be confirmed by elucidating genotoxicity of the compound by use of any one or two or more of these methods.

The compound of the formula (1) according to a certain aspect can be confirmed to be safe as an active ingredient in the pharmaceutical composition by conducting, for example, a skin sensitization test. The skin sensitization test includes skin sensitization tests using guinea pigs, such as the Buehler method (Buehler, E. V., Arch. Dermatol., 91, pp. 171-177, 1965), the GPMT method (maximization method; Magnusson B., et al., J. Invest. Dermatol., 52, pp. 268-276, 1969), and the APT method (adjuvant and patching method (Sato, Y. et al., Contact Dermatitis, 7, pp. 225-237, 1981)). This test also includes skin sensitization tests using mice such as the LLNA (local lymph node assay) method (OECD Guideline for the testing of chemicals 429, skin sensitization 2002; Takeyoshi, M. et al., Toxicol. Lett., 119 (3), pp. 203-8, 2001; and Takeyoshi, M. et al., J. Appl. Toxicol., 25 (2), pp. 129-34, 2005). The safety as an active ingredient in the pharmaceutical composition can be confirmed by elucidating skin sensitizing properties of the compound by use of any one or two or more of these methods.

The compound of the formula (1) according to a certain aspect can be confirmed to be safe as an active ingredient in the pharmaceutical composition by conducting, for example, a skin photosensitization test. Examples of the skin photosensitization test include a skin photosensitization test using guinea pigs (see “Drug Nonclinical Test Guideline Commentary 2002”, Yakuji Nippo, published on 2002, 1-9: Skin Photosensitization Test, etc.). Examples of the method include the adjuvant and strip method (Ichikawa, H. et al., J. Invest. Dermatol., 76, pp. 498-501, 1981), the Harber method (Harber, L. C., Arch. Dermatol., 96, pp. 646-653, 1967), the Horio method (Horio, T., J. Invest. Dermatol., 67, pp. 591-593, 1976), the Jordan method (Jordan, W. P., Contact Dermatitis, 8, pp. 109-116, 1982), the Kochever method (Kochever, I. E. et al., J. Invest. Dermatol., 73, pp. 144-146, 1979), the Maurer method (Maurer, T. et al., Br. J. Dermatol., 63, pp. 593-605, 1980), the Morikawa method (Morikawa, F. et al., “Sunlight and Man”, Tokyo Univ. Press, Tokyo, pp. 529-557, 1974), and Vinson method (Vinson, L. J., J. Soc. Cosm. Chem., 17, pp. 123-130, 1966). The safety as an active ingredient in the pharmaceutical composition can be confirmed by elucidating skin photosensitizing properties of the compound by use of any one or two or more of these methods.

The compound of the formula (1) according to a certain aspect can be confirmed to be safe as an active ingredient in the pharmaceutical composition by conducting, for example, a local irritation test. Examples of the local irritation test include irritation tests at subcutaneous administration sites using rats, monkeys, or other animal species. Such methods involving evaluating irritation by the observation of general conditions at an administration site after single or repetitive subcutaneous doses or the autopsy and pharmacological histological examination of the skin or subcutaneous tissues. The safety as an active ingredient in the pharmaceutical composition can be confirmed by elucidating local irritation of the compound by use of any one or two or more of these methods.

The compound of the formula (1) according to a certain aspect can be confirmed to be safe as an active ingredient in the pharmaceutical composition by conducting, for example, a hemolysis test. Examples of the hemolysis test include a method using erythrocytes collected from rats, humans, or the like (Greco, et al., Sci Rep 10, 13206, 2020). Such a method involves evaluating the hemolysis of erythrocytes mixed with the compound as an index for cell membrane damaging properties. The safety as an active ingredient in the pharmaceutical composition can be confirmed by elucidating cell membrane damaging properties of the compound by use of such a method.

The compound of the formula (1) according to a certain aspect can be confirmed to be safe as an active ingredient in the pharmaceutical composition by conducting, for example, a safety pharmacology test for the cardiovascular system. Examples of the safety pharmacology test for the cardiovascular system include the telemetry method (method for measuring the influence of administration of the compound without anesthetization on electrocardiograms, heart rates, blood pressures, blood streams, and the like (Electrocardiogra Echocardiographic, Blood Pressure and Pathological Tests of Animals for Fundamental and Clinical Medicine, edited by Shigeru Sugano, Hirokazu Tsubone, and Yoshinori Nakada, published on 2003, MARUZEN-YUSHODO Co., Ltd.), the APD method (method for measuring cardiac muscle cell action potential retention times (Muraki, K. et al., A M. J. Physiol., 269, H524-532, 1995; and Ducic, I. et al., J. Cardiovasc. Pharmacol., 30(1), pp. 42-54, 1997)), the hERG inhibition evaluation method (patch clamping method (Chachin, M. et al., Nippon Yakurigaku Zasshi, 119, pp. 345-351, 2002), the binding assay method (Gilbert, J. D. et al., J. Pharm. Tox. Methods, 50, pp. 187-199, 2004), the Rb⁺ efflux assay method (Cheng, C. S. et al., Drug Develop. Indust. Pharm., 28, pp. 177-191, 2002), and the membrane potential assay method (Dorn, A. et al., J. Biomol. Screen., 10, pp. 339-347, 2005). The safety as an active ingredient in the pharmaceutical composition can be confirmed by elucidating action of the compound on the cardiovascular system by use of any one or two or more of these methods.

The compound of the formula (1) according to a certain aspect can be confirmed to be safe as an active ingredient in the pharmaceutical composition by conducting, for example, a safety pharmacology test for the central nervous system. Examples of the safety pharmacology test for the central nervous system include the FOB method (functional observational battery (Mattson, J. L. et al., J. American College of Technology, 15 (3), pp. 239-254, 1996)) and a modified version of the Irwin method (method for evaluating general symptoms and behavior observation (Irwin, S., Comprehensive Observational Assessment (Berl.) 13, pp. 222-257, 1968)). The safety as an active ingredient in the pharmaceutical composition can be confirmed by elucidating action of the compound on the central nervous system by use of any one or two or more of these methods.

The compound of the formula (1) according to a certain aspect can be confirmed to be safe as an active ingredient in the pharmaceutical composition by conducting, for example, a safety pharmacology test for the respiratory system. Examples of the safety pharmacology test for the respiratory system include the measurement method using a respiratory function measurement apparatus (measurement of respiration rates, single ventilation volumes, minute ventilations, and the like) (Drorbaugh, J. E. et al., Pediatrics, 16, pp. 81-87, 1955; and Epstein, M. A. et al., Respir. Physiol., 32, pp. 105-120, 1978), and the measurement method using a blood gas analyzer (measurement of blood gas, hemoglobin oxygen saturation, and the like) (Matsuo, S., Medicina, 40, pp. 188-, 2003). The safety as an active ingredient in the pharmaceutical composition can be confirmed by elucidating action of the compound on the respiratory system by use of any one or two or more of these methods.

The compound of the formula (1) according to a certain aspect can be confirmed to be safe as an active ingredient in the pharmaceutical composition by conducting, for example, a general toxicity test. The general toxicity test is a method of subcutaneously or intravenously administering the compound dissolved or suspended in an appropriate solvent at single or repetitive doses (for several days) to rodents such as rats or mice or non-rodent animals such as monkeys or dogs, and thereby evaluating observation of general conditions, clinicochemical change, pathohistological change, and the like of the recipient animals. The safety as an active ingredient in the pharmaceutical composition can be confirmed by elucidating general toxicity of the compound by use of such a method.

The compound of the formula (1) according to a certain aspect can be confirmed to be safe as an active ingredient in the pharmaceutical composition by conducting, for example, a reproductive and developmental toxicity test. The reproductive and developmental toxicity test is a test for studying the induction of a harmful effect of the compound on the reproductive and developmental processes using rodents such as rats or mice or non-rodent animals such as monkeys or dogs (see “Drug Nonclinical Test Guideline Commentary 2002”, Yakuji Nippo, published on 2002, 1-6: Reproductive and Developmental Toxicity Test, etc.). Examples of the reproductive and developmental toxicity test include tests on fertility and early embryogenesis up to nidation, tests on development and maternal functions before and after birth, and tests on embryogenesis and fetal development (see IYAKUSHIN No. 1834, 2000, Appendix, “Guideline for Drug Toxicity Test”, [3] Reproductive and Developmental Toxicity Test, etc.). The safety as an active ingredient in the pharmaceutical composition can be confirmed by elucidating reproductive and developmental toxicity of the compound by use of such a testing method.

The compound of the formula (1) according to a certain aspect can be confirmed to be safe as an active ingredient in the pharmaceutical composition by conducting, for example, a cytochrome P450 enzyme inhibition or induction test (Gomez-Lechon, M. J. et al., Curr. Drug Metab., 5 (5), pp. 443-462, 2004). Examples of the cytochrome P450 enzyme inhibition or induction test include a method of determining in vitro whether the compound inhibits activity of the cytochrome P450 enzyme using cytochrome P450 enzyme of each molecular species purified from cells or prepared using a genetic recombinant, or human P450 expression system microsomes (Miller, V. P. et al., Ann. N.Y. Acad. Sci., 919, pp. 26-32, 2000), a method of measuring change in expression of cytochrome P450 enzyme of each molecular species or its enzymatic activity using human liver microsomes or cell homogenates (Hengstler, J. G. et al., Drug Metab. Rev., 32, pp. 81-118, 2000), and a method of extracting RNA from human hepatocytes exposed to the compound, and comparing an mRNA expression level with a control to examine the ability of the compound to induce the enzyme (Kato, M. et al., Drug Metab. Pharmacokinet., 20 (4), pp. 236-243, 2005). The safety as an active ingredient in the pharmaceutical composition can be confirmed by elucidating action of the compound on the inhibition or induction of the cytochrome P450 enzyme by use of any one or two or more of these methods.

The compound of the formula (1) according to a certain aspect can be confirmed to be safe as an active ingredient in the pharmaceutical composition by conducting, for example, a reactive metabolite production confirmation test. Examples of the reactive metabolite production confirmation test include a method of incubating the compound with human liver microsomes in the presence of NADPH and glutathione fluorescently labeled with a dansyl group (dGSH), trapping reactive metabolites as dGSH adducts, and comprehensively detecting peaks ascribable to the reactive metabolites from the amounts of dGSH adducts produced with fluorescence intensity as an index (Junping Gan. et al., Chem. Res. Toxicol. 2005, 18, 896-903), and a method of incubating a 14C-labeled form of the compound with human liver microsomes in the presence of NADPH and measuring radioactivity of the compound covalently bound to protein (Baillie T. A., Drug Metabolizing Enzymes. Cytochrome P450 and Other Enzymes in Drug Discovery and Development, pp. 147-154, 2003). The safety as an active ingredient in the pharmaceutical composition can be confirmed by elucidating risk of generation of idiosyncratic drug toxicity caused by the reactive metabolite production of the compound by use of any one or two or more of these methods.

The compound of the formula (1) according to a certain aspect can be confirmed to be useful as an active ingredient in the pharmaceutical composition by conducting, for example, a membrane permeability test. Examples of the membrane permeability test include a method of measuring cell membrane permeability of the compound in a cell culture system using Caco-2 cells (Delie, F. et al., Crit. Rev. Ther. Drug Carrier Syst., 14, pp. 221-286, 1997; Yamashita, S. et al., Eur. J. Pham. Sci., 10, pp. 195-204, 2000; and Ingels, F. M. et al., J. Pham. Sci., 92, pp. 1545-1558, 2003), a method of measuring cell membrane permeability of the compound in a cell culture system using MDCK cells (Irvine, J. D. et al., J. Pham. Sci., 88, pp. 28-33, 1999), and a method of measuring membrane permeability of the compound using artificial lipid membranes (Wohnsland, F. et al., J. Med. Chem., 44, pp. 923-930, 2001). The usefulness as an active ingredient in the pharmaceutical composition can be confirmed by elucidating cell permeability of the compound by use of any one or two or more of these methods.

The compound of the formula (1) according to a certain aspect can be confirmed to be useful as an active ingredient in the pharmaceutical composition by conducting, for example, an evaluation test on interaction involving a drug transporter. Examples of the evaluation test on interaction involving a drug transporter include a method of evaluating the interaction of the compound with P-gp using MDCKII cells forced to express MDR1 (Poirier, A. et al., Drug Metab. Dispos., 42 (9), pp. 1411-1422, 2014). Another example thereof includes a method of evaluating the interaction of the compound with OAT1 using CHO cells forced to express OAT1 (Juhasz, V. et al., J. Pharm. Sci., 102 (5), pp. 1683-1687, 2013). The usefulness as an active ingredient in the pharmaceutical composition can be confirmed by elucidating interaction of the compound with an ATP-binding cassette (ABC) transporter or a solute carrier (SLC) transporter by use of any one or two or more of these methods.

The compound of the formula (1) according to a certain aspect can be confirmed to be useful as an active ingredient in the pharmaceutical composition by conducting, for example, a blood concentration transition measurement test. Examples of the blood concentration transition measurement test include a method of administering the compound to rodents, monkeys, or dogs, and then measuring transition of the concentration of the compound in blood by use of LC-MS/MS (“Newest Mass Spectrometry for Life Science”, Kodansha Scientific, 2002, edited by Kenichi Harada et al, etc.). The usefulness as an active ingredient in the pharmaceutical composition can be confirmed by elucidating blood concentration transition of the compound by use of such a method.

The compound of the formula (1) according to a certain aspect can be confirmed to be useful as an active ingredient in the pharmaceutical composition by conducting, for example, a metabolism test. Examples of the metabolism test include the metabolic stability test method (method of calculating a metabolic rate of the compound in liver microsomes of humans or other animal species (see Shou, W. Z. et al., J. Mass Spectrom., 40 (10) pp. 1347-1356, 2005; L1, C. et al., Drug Metab. Dispos., 34 (6), 901-905, 2006, etc.), the blood (plasma) stability test method, the metabolite molecular species test method, and the reactive metabolite production confirmation test method. The usefulness as an active ingredient in the pharmaceutical composition can be confirmed by elucidating a metabolic profile of the compound by use of any one or two or more of these methods.

The compound of the formula (1) according to a certain aspect can be confirmed to be useful as an active ingredient in the pharmaceutical composition by conducting, for example, a solubility test. The testing method is not particularly limited, and a flask shaking method can be used as an example. The type of a solvent, pH, and other compositional profiles, for example, the type or concentration of a buffer solution or a surfactant, are determined, and a sufficient amount of the compound is added to such a component to obtain a saturated solution. The concentration of the compound in this saturated solution is measured by liquid chromatography, ultraviolet-visible absorption spectroscopy, or any of other appropriate methods (see US Pharmacopeia (2024) <1236> Solubility measurements, etc.). The usefulness as an active ingredient in the pharmaceutical composition can be confirmed by elucidating solubility of the compound by use of such a method.

The compound of the formula (1) according to a certain aspect can be confirmed to be useful as an active ingredient in the pharmaceutical composition by examining, for example, renal dysfunction. Examples of the pharmacological test for kidney functions include the renal blood flow and glomerular filtration rate measurement method [Physiology, 18th edition, Bunkodo Co., Ltd., 1986, Chapter 17]. The usefulness as an active ingredient in the pharmaceutical composition can be confirmed by elucidating action of the compound on kidney functions by use of any one or two or more of these methods.

The pharmaceutical composition may further include an additional pharmaceutically acceptable component other than the compound of the formula (1) described above without impairing the effects of embodiments. Examples of the additional component include excipients, fluidizers, surfactants, suspending agents, emulsifiers, preservatives, colorants, fragrances, sweeteners, and corrigents. One or two or more known components in combination can be appropriately used as the additional component.

The pharmaceutical composition according to the present embodiment can be subcutaneously administered or intravenously administered and is preferably subcutaneously administered. The dosage form is not particularly limited as long as the dosage form is suitable for subcutaneous administration or intravenous administration. For example, an injection, an injectable filler, or an intravenous drip can be used.

The pharmaceutical composition according to the present embodiment can be used in the treatment of the disease related to the inhibition of TNFR1 described above. In this context, the “treatment” includes suppression of progression of the disease, remission of symptoms of the disease, and cure of the disease.

### [Treatment Method]

An embodiment of the present invention may be a method for treating a disease, including administering the compound of the formula (1) described above or the pharmaceutical composition including the same to a subject.

In the method according to the present embodiment, preferably, the disease is the disease related to the inhibition of TNFR1 described above, and the subject has the disease related to the inhibition of TNFR1 described above. The administration is subcutaneous administration or intravenous administration, preferably subcutaneous administration.

### EXAMPLES

Hereinafter, the present invention will be further specifically described with reference to Examples and Test Examples. However, the present invention is not limited to these examples by any means. Changes or modifications may be made therein without departing from the scope of the present invention. Compounds shown in Reference Examples and Examples given below do not necessarily abide by the IUPAC nomenclature. For brief description, abbreviations described in Table 1 below may be used.

TABLE 1

| Abbreviation | Structure |
|---|---|
| 3Py56dOMe | 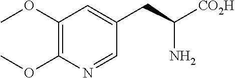 [SMILES: COc1cc(C[C@H](N)C(=O)O)cnc1OC] |
| C | 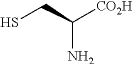 |
| cC12COO | 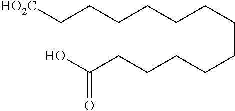 [SMILES: O=C(O)CCCCCCCCCCCCC(=O)O] |
| CeG | 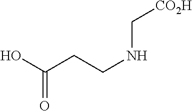 |
| ClAc | 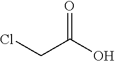 |
| DapAc | 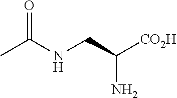 |
| E | 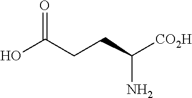 |
| F34dOMe | 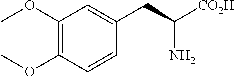 [SMILES: COc1ccc(C[C@H](N)C(=O)O)cc1OC] |
| F4COO | 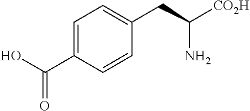 [SMILES: N[C@@H](Cc1ccc(C(=O)O)cc1)C(=O)O] |
| H2PyOMe | 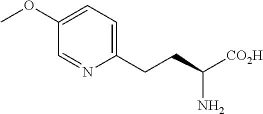 |
| H3Pyrd6OMe | 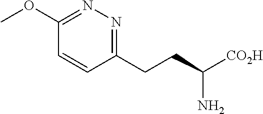 |
| MeA | 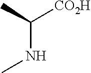 |
| MeD | 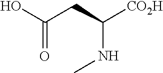 |
| MeE | 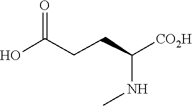 |
| MeG | 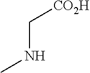 |
| MeK | 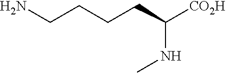 |
| NH2 | H—NH₂ |
| OH | H—OH |
| Q | 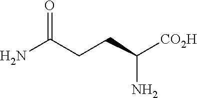 |
| Q4Pye | 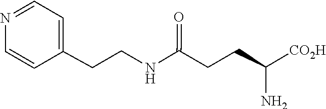 [SMILES: N[C@@H](CCC(=O)NCCc1ccncc1)C(=O)O] |
| QaEt | 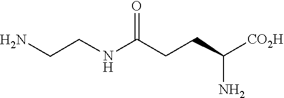 [SMILES: NCCNC(=O)CC[C@H](N)C(=O)O] |
| QaMe3Az | 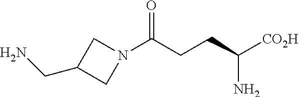 [SMILES: NCC1CN(C(=O)CC[C@H](N)C(=O)O)C1] |
| QaPr | 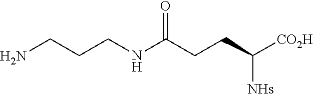 |
| Qpyrli3RMe | 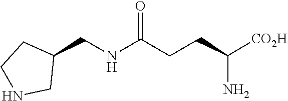 [SMILES: N[C@@H](CCC(=O)NC[C@@H]1CCNC1)C(=O)O] |
| S | 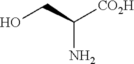 |
| W | 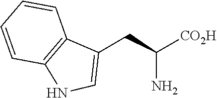 [SMILES: N[C@@H](Cc1c[nH]c2ccccc12)C(=O)O] |
| W72Py | 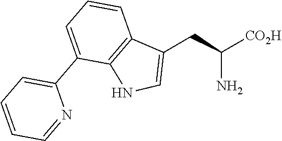 [SMILES: N[C@@H](Cc1c[nH]c2c(-c3ccccn3)cccc12)C(=O)O] |
| W72Py5CON | 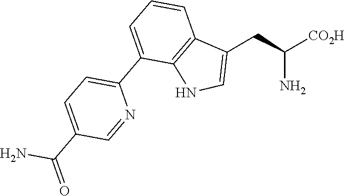 [SMILES: NC(=O)c1ccc(-c2cccc3c(C[C@H](N)C(=O)O)c[nH]c23)nc1] |
| W72Py5COO | 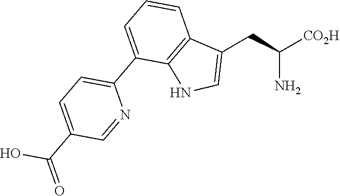 [SMILES: N[C@@H](Cc1c[nH]c2c(-c3ccc(C(=O)O)cn3)cccc12)C(=O)O] |
| W72pyrm | 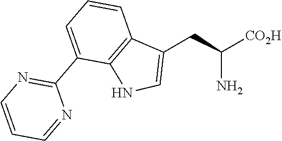 [SMILES: N[C@@H](Cc1c[nH]c2c(-c3ncccn3)cccc12)C(=O)O] |
| W72Pyrm5F | 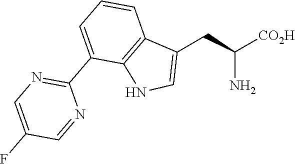 [SMILES: N[C@@H](Cc1c[nH]c2c(-c3ncc(F)cn3)cccc12)C(=O)O] |
| W73Pyrd | 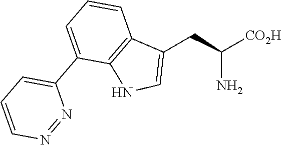 [SMILES: N[C@@H](Cc1c[nH]c2c(-c3cccnn3)cccc12)C(=O)O] |
| W73Pyz1Me | 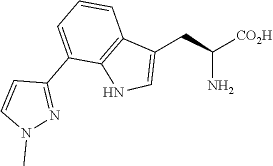 [SMILES: Cn1ccc(-c2cccc3c(C[C@H](N)C(=O)O)c[nH]c23)n1] |
| W7C | 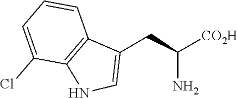 [SMILES: N[C@@H](Cc1c[nH]c2c(Cl)cccc12)C(=O)O] |
| Y | 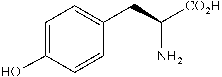 [SMILES: N[C@@H](Cc1ccc(O)cc1)C(=O)O] |

### Abbreviation (General)

AA: amino acid;
Alloc: allyloxycarbonyl;
Allyl: allyl;
Boc: tertiary butoxycarbonyl;
ClAc: chloroacetyl;
ClAcOH: chloroacetic acid;
ClAcOSu: N-(chloroacetoxy) succinimide;
conc: concentration;
DCM: dichloromethane;
DIC or DIPCI: N,N′-diisopropylcarbodiimide;
DIEA or DIPEA: N, N-diisopropylethylamine;
DMF: N, N-dimethylformamide;
DMSO: dimethyl sulfoxide;
DODT: 6-dioxa-1,8-octane-dithiol;
EDCI: 1-(3-dimethylaminopropyl)-3-ethylcarbodiimide;
ESI-MS: electrospray ionization-mass spectrometry;
Et2O: diethyl ether;
Et2NH: diethylamine
Fmoc: 9-fluorenylmethyloxycarbonyl;
g: gram (unit);
h: hour (unit);
H₂O: water;
HATU: O-(7-azabenzotriazol-1-yl)-N, N,N′,N′-tetramethyluronium hexafluorophosphate;
HCl: hydrogen chloride;
HCTU: O-(6-chlorobenzotriazol-1-yl)-N, N,N′,N′-tetramethyluronium hexafluorophosphate;
HFIP: 1,1,1,3,3,3-hexafluoro-2-propanol;
HOAt: 1-hydroxybenzotriazole;
HOBt: 2,2,4,6,7-pentamethyldihydrobenzofuran-5-sulfonyl;
HOSu: N-hydroxysuccinimide;
HPLC: high-performance liquid chromatography;
IPA: 2-propanol;
L: litter (unit);
LC-MS or LC/MS: liquid chromatography-mass spectrometer;
M: mole (unit);
MeCN or CH₃CN: acetonitrile;
MeOH: methanol;
mg: milligram (unit);
min: minute (unit);
mL: milliliter (unit);
μL: microliter (unit);
mM: millimole (unit);
μM: micromole (unit);
mmol: millimole (unit);
mm: millimeter (unit);
μm: micrometer (unit);
Mmt: monomethoxytrityl;
Mpe: 3-(3-methyl) pentyl;
m/z: mass-charge ratio;
nM: nanomole (unit);
nm: nanometer (unit);
NMP: N-methyl-2-pyrrolidone;
ODS: octadecyl-bound column;
Osu: succinimide;
Oxyma pure: ethyl cyano (hydroxyimino)acetate;
rpm: rotation per minute (unit);
tert: tertiary;
tBu: tertiary butyl;
TEA: triethylamine;
TEAA: triethylamine acetate;
TFA: trifluoroacetic acid;
THF: tetrahydrofuran;
TIS: triisopropylsilane;
Trt or Tr: trityl;
UV: ultraviolet;
Xan: 9-xanthenyl.

Starting materials, building blocks, reagents, acids, bases, solid-phase resins, and solvents used in the chemical synthesis of the compounds according to Examples were commercially available products as is or can be synthesized by those skilled in the art using organic chemical approaches.

Peptide residues are counted by counting a ClAc-amino acid residue as the first residue, followed by the second residue, the third residue, and the like toward a resin. General amino acids and starting materials used are listed below, and side chain protective groups are shown within parentheses.

Fmoc-3Py56dOMe-OH; Fmoc-C(Mmt)-OH; Fmoc-C(Trt)-OH; HO-cC12COO-tBu; Fmoc-CeG (tBu)-OH; ClAc—OH; ClAc—NHS; Fmoc-DapAc-OH; Fmoc-E (Allyl)-OH; Fmoc-E-OtBu; Fmoc-F34dOMe-OH; Fmoc-F4COO(tBu)-OH; Fmoc-H2PyOMe-OH; Fmoc-H3Pyrd6OMe-OH; Fmoc-MeA-OH; Fmoc-MeD (tBu)-OH; Fmoc-MeD (Mpe)-OH; Fmoc-MeE (tBu)-OH; Fmoc-MeG-OH; Alloc-Mek (Fmoc)-OH; Fmoc-Q (Trt)-OH; Fmoc-Q (Xan)-OH; Fmoc-QaEt (Boc)-OH; Fmoc-QaMe3Az (Boc)-OH; Fmoc-QaPr (Boc)-OH; Fmoc-Qpyrli3RMe (Boc)-OH; Fmoc-S(tBu)-OH; Fmoc-S(Trt)-OH; Fmoc-W(Boc)-OH; Fmoc-W72Py-OH; Fmoc-W72Py5CON—OH; Fmoc-W72Py5COO(Boc, tBu)-OH; Fmoc-W72pyrm (Boc)-OH; Fmoc-W72Pyrm5F(Boc)-OH; Fmoc-W73Pyrd (Boc)-OH; Fmoc-W73pyz1Me (Boc)-OH; Fmoc-W7C—OH; Fmoc-Y (tBu)-OH.

A peptide chain on a solid-phase resin was elongated by the Fmoc method using a resin described in each Example as a starting material and using peptide coupling reaction conditions and Fmoc removal reaction conditions usually used. A peptide of interest was synthesized, for example, by using Sieber amide resin (Watanabe Chemical Industries, Ltd.), starting by removal of the Fmoc group, and repeating introduction of each Fmoc-amino acid and deprotection of the Fmoc group. In this respect, the elongation of the peptide chain was performed by automated synthesis, and the synthesis was performed using Liberty PRIME or Liberty Blue from CEM Corp. or Syro I or Syro II from Biotage Japan Ltd. as a solid-phase synthesizer in accordance with manufacturer's manual, unless otherwise specified. For the introduction of each residue, double coupling which repetitively performs peptide coupling reaction twice was performed, if necessary. On the other hand, some amino acids were introduced by isolating the resin from automated synthesizer and performing reaction using a reagent or the like prepared before use (manual coupling).

For example, approximately 4 to 11 equivalents of an Fmoc-amino acid, approximately 8 to 20 equivalents of DIC, and approximately 4 to 10 equivalents of Oxyma pure, or approximately 4 to 6 equivalents of an Fmoc-amino acid, approximately 8 to 13 equivalents of DIEA, and approximately 4 to 6 equivalents of HATU were added to a peptide chain, from which Fmoc was removed, supported on a solid-phase resin in DMF, NMP, DMF/NMP, or DMF/DCM, and shaken under any of reaction conditions shown in Table 2 below, and this step was performed once or twice, followed by the washing of the resin with DMF.

TABLE 2

| Method | Temperature (° C.) | Time (min) |
|---|---|---|
| 1-1 | Room temperature | 30-360 |
| | (10-30° C.) | |
| 1-2 | 40-50 | 10-30 |
| 1-3 | 75 | 10-30 |
| 1-4 | 90 | 3-15 |
| 1-5 | 105 | 2-10 |

The Fmoc group was removed in accordance with, for example, the following method: a peptide chain supported on a solid-phase resin, after introduction of an Fmoc-amino acid, was shaken under any of reaction conditions shown in Table 3 below, and this step was performed once or twice, followed by washing of the resin with DMF.

TABLE 3

| Method | Reagent | Temperature | Time | |
|---|---|---|---|---|
| 2-1 | 20% solution of piperidine in DMF | 50-90° C. | 1-5 | min |
| 2-2 | 20% solution of piperidine in DMF | Room | 1-15 | min |
| | | temperature | | |
| 2-3 | Solution of 4% pyrrolidine/83 mM Oxyma | 110° C. | 1-5 | min |
| | pure in DMF | | | |
| 2-4 | 10% solution of pyrrolidine in DMF | 50-90° C. | 1-5 | min |
| 2-5 | 10% solution of pyrrolidine in DMF | Room | 1-15 | min |
| | | temperature | | |
| 2-6 | Solution of 5% piperazine/0.1M HOBt in | 50-90° C. | 1-5 | min |
| | DMF | | | |
| 2-7 | Solution of 5% piperazine/0.1M HOBt in | Room | 1-15 | min |

| | |
|---|---|
| DMF | temperature |

The introduction of each Fmoc-amino acid and the deprotection of the Fmoc group were repeated in accordance with the method described above for elongation into a peptide chain of interest, followed by introduction of a chloroacetyl group. The introduction method is any method of Table 4 below. The method was performed by, after removal of the Fmoc group, adding a solution containing approximately 4 to 10 equivalents of chloroacetic acid, approximately 4 to 10 equivalents of a condensing agent, and approximately 4 to 10 equivalents of an additive in a solvent given below to the solid-phase resin carrying the Fmoc-protected peptide obtained in the preceding step, or, after removal of the Fmoc group, adding a solution containing approximately 4 to 10 equivalents of N-(chloroacetoxy) succinimide in a solvent given below to the solid-phase resin carrying the Fmoc-protected peptide, and shaking the mixture at room temperature for 30 to 60 minutes.

TABLE 4

| Method | Condensing agent | Additive | Solvent |
|---|---|---|---|
| 3-1 | DIC | HOSu | DMF/DCM |
| 3-2 | HATU | DIPEA | DMF |
| 3-3 | DIC | HOSu | DCM/NMP |
| 3-4 | HCTU | DIPEA | DMF |
| 3-5 | HOAt | DIPEA | DMF |
| 3-6 | ClAcOSu | None | DMF |
| 3-7 | DIC | Oxyma pure | DMF |

The deprotection of a side chain and excision from the solid-phase resin were performed by adding any reactant cocktail (TFA/H₂O/TIS/DODT mixture or HFIP/DCM) of Table 5 below to the peptide bound to the resin and shaking the mixture at room temperature for 30 to 120 minutes.

TABLE 5

| Method | Reactant cocktail |
|---|---|
| 4-1 | TFA/H₂O/TIS/DODT(v/v/v/v) = 92.5/2.5/2.5/2.5 |
| 4-2 | TFA/H₂O/TIS/DODT(v/v/v/v) = 90/2.5/2.5/5 |
| 4-3 | HFIP/DCM(v/v) = 1/4 |

Then, the reaction solution was collected by filtration from the frit, and the filtrate was added to an excess of diisopropyl ether, diethyl ether, or a mixed solvent of diisopropyl ether and hexane or diethyl ether and hexane (e.g., 1/1, v/v) cooled to 0° C. so that cloudy precipitation occurred. Centrifugation was performed in order to obtain this precipitate solid, and the supernatant was decanted. The obtained solid was washed again with a small amount of diisopropyl ether, diethyl ether, or a mixed solvent of diisopropyl ether and hexane or diethyl ether and hexane (e.g., 1/1, v/v) cooled to 0° C., and then dried under reduced pressure.

The solid thus obtained was used in peptide cyclization reaction. For the cyclization reaction of the peptide, the peptide was dissolved in DMSO or a mixed solvent of DMSO/H₂O, MeCN/H₂O, DMSO/IPA/H₂O, or DMSO/MeCN/H₂O such that the final concentration of the peptide was 2 mM to 5 mM based on the number of moles of the solid-phase resin used. Then, 5 to 35 equivalents of triethylamine were added to the solution, and the mixture was shaken at room temperature overnight or for 2 hours. The obtained reaction solution was concentrated under reduced pressure and then purified.

A method for purifying the obtained residue or partially purified peptide involved using AutoPurification System from Waters Corp., AutoPurification System with SQD from Waters Corp., Preparative HPLC System from Waters Corp., or Shimazu prep-HPLC System (LC-20AP, SPDM20A, CTO-20AC, and CBM-20A) as a reverse-phase preparative purification apparatus and eluting the compound of interest while monitoring m/z ions derived from the compound, unless otherwise specified. Mass spectra obtained in an ESI-positive scan mode were confirmed to be consistent with mass spectra including polyvalent ions calculated from the molecular formula of the compound of interest, within the error range of the mass spectrometer used. Purification conditions involving the column used were any condition of Table 6 below, under which purification was performed.

TABLE 6

| | | Mobile | Mobile | | | |
|---|---|---|---|---|---|---|
| Method | Column | phase A | phase B | Temperature | Flow rate | |
| P-A | Waters XBridge(R) C18 | 0.1% TFA | 0.1% TFA in | 40° C. or | 17 | mL/min |
| | 5 μm, 19 × 150 mm | in H₂O | CH₃CN | 60° C. | | |
| P-B | Waters XBridge C18 | 0.1% TFA | 0.1% TFA in | 40° C. or | 45-120 | mL/min |
| | 5 μm, 30 × 150 mm | in H₂O | CH₃CN | 50° C. | | |
| P-C | Waters XBridge C18 | 0.1% TFA | 0.1% TFA in | 40° C. | 20-120 | mL/min |
| | 5 μm, 50 × 150 mm | in H₂O | CH₃CN | | | |
| P-D | Phenomenex Kinetex | 1.0% AcOH | 1.0% AcOH | 40° C. | 45 | mL/min |

| | | |
|---|---|---|
| EVO C18 5 μm, 30 × | in H₂O | in CH₃CN |
| 150 mm | | |

For the structural determination of the chemically synthesized peptide, a molecular weight calculated in consideration of the amino acids used and building blocks optionally used according to the sequence of interest was confirmed by ESI-MS(+) in mass spectrometry. The “ESI-MS(+)”refers to electrospray ionization-mass spectrometry carried out in a positive ion mode. The detected mass was reported by “m/z” unit notation. A compound having a molecular weight of larger than approximately 1000 was highly frequently detected as divalent or trivalent ions.

For the mass spectrometry of peptides synthesized in Examples given below, analysis was conducted using Shimazu LC/MS system (LC-20AD, CTO-20AC, SPD-M20A, SIL-20AHT, CBM-20A and LCMS-2020) or Shimazu LC/MS system (LC-20ADXR, CTO-20AC, SPD-M20A, SIL-20AXR, CBM-20A and LCMS-2020) as an analysis apparatus under any condition of Table 7 below, unless otherwise specified.

Column: Kinetex EVO C18 2.6 μm, 2.1 ID×150 mm, 100 angstroms

Mobile phase: A=0.025% TFA in H₂O; B=0.025% TFA in CH₃CN

Detection: UV wavelength 225 nm

TABLE 7

| Method | Temperature | Flow rate | | Gradient (% B conc) |
|---|---|---|---|---|
| Q-A | 60° C. | 0.5 | mL/min | 5-45/ 7.15 min, 45-95/ 0.3 min |
| Q-B | 60° C. | 0.5 | mL/min | 20-60/ 7.15 min, 60-95/ 0.3 min |
| Q-C | 60° C. | 0.25 | mL/min | 20-60/ 20.0 min, 60-95/ 1.0 min |
| Q-D | 40° C. | 0.25 | mL/min | 20-60/ 20.0 min, 60-95/ 1.0 min |

Hereinafter, specific structures and the like of the synthesized peptides will be shown.

### Synthesis of Compound According to Example 1 Sequence: ClAc—H2PyOMe-Y—S—W-MeA-Qpyrli3RMe-F4COO—W7C—S—Y—F34dOMe-MeG-W72Py-C—NH2

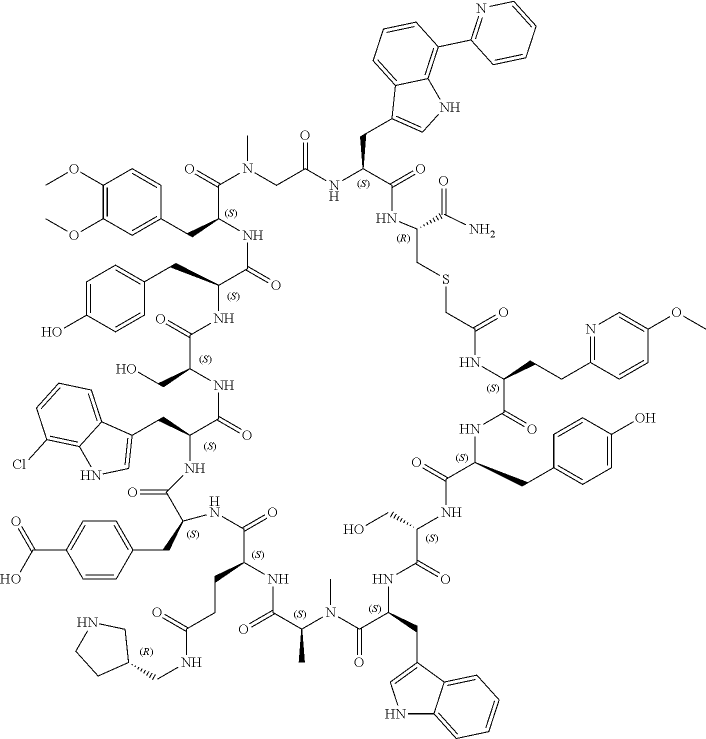

Elongation into the peptide chain of interest was performed in accordance with the general method described above. Specifically, the peptide of interest was synthesized, starting at the removal of the Fmoc group, by the general method mentioned above using Sieber amide resin (Watanabe Chemical Industries, Ltd., 0.47 mmol/g, 0.11 g). This synthesis was performed using Liberty Blue from CEM Corp. as a solid-phase synthesizer in accordance with manufacturer's manual. Each residue was introduced using Fmoc-AA/DIC/Oxyma pure (5.25 equivalents/10 equivalents/5 equivalents) per equivalent of the resin, and reaction was performed under conditions of the method 1-1, 1-2, or 1-4. Specifically, the reaction was basically performed once at 90° C. for 3 minutes in DMF under microwave irradiation. However, the reaction was performed once at 40° C. for 30 minutes for the first residue. The reaction was performed twice at 90° C. for 3 minutes for the fourth residue and the 11th residue. The reaction was performed once at room temperature for 45 minutes for the 13th residue. The reaction was performed once at 50° C. for 15 minutes for the 14th residue. Fmoc was removed through reaction with a 20% solution of piperidine in DMF at 75° C. for 3 minutes in accordance with the method 2-1 or 2-2. However, the Fmoc removal for the fourth residue and the 11th residue was performed through reaction at room temperature for 5 minutes and subsequent reaction for 5 minutes. The chloroacetyl group was introduced by, after removal of the Fmoc group of an α-amino group by the method mentioned above, adding chloroacetic acid (approximately 5 equivalents), HATU (approximately 5 equivalents), and DIPEA (approximately 10 equivalents) to the solid-phase resin carrying the Fmoc-protected peptide obtained in the preceding step in accordance with the method 3-2, and shaking the mixture at room temperature for 30 minutes in DMF. For the deprotection of a side chain and excision from the solid-phase resin, first, the resin obtained after the chloroacetyl group introduction step was washed with DMF and DCM, then washed with diethyl ether, and then dried under reduced pressure. Subsequently, the reactant cocktail (2.5 mL, TFA/H2O/TIS/DODT mixture at a volume ratio of 92.5/2.5/2.5/2.5) was added to a reaction container containing the solid-phase resin in accordance with the method 4-1, and shaken at room temperature for 90 minutes. The reaction solution was collected by filtration from the frit. The solid-phase resin remaining in the reaction container was shaken again with the cocktail for excision, and a solution component was collected from the frit and mixed with the filtrate mentioned above. This filtrate was added to an excess of a diethyl ether/hexane (1/1) mixed solvent cooled to 0° C. so that cloudy precipitation occurred. This mixture was centrifuged (9000 rpm, 1 min), and the solution was decanted. The obtained solid was washed again with a small amount of diethyl ether cooled to 0° C., and then dried under reduced pressure. The obtained solid was used in subsequent cyclization reaction. For the cyclization reaction of the peptide, the peptide was dissolved in DMSO such that the final concentration of the peptide was 5 mM based on the number of moles of the solid-phase resin used. Then, 6 equivalents of triethylamine were added to the solution, and the mixture was stirred overnight at room temperature. Then, the reaction solution was concentrated under reduced pressure.

The obtained crude product was purified under the following conditions (column: Waters Xbridge (R) C18 5 μm 50×150 mm; mobile phase: A=0.1% TFA in H₂O, B=0.1% TFA in MeCN; temperature: 40° C.; gradient (% B): 5-26% over 3 minutes, subsequently 26-31% over 8 minutes, and subsequently 31-60% over 1 minute; flow rate: 120 mL/min).

The purity of the compound of interest was calculated from an area ratio of an LC/MS (UV wavelength: 225 nm) chromatogram obtained under the following analysis condition and was consequently 93.4%.

Analysis condition Q-D: retention time=9.49 min

ESI-MS(+) found m/z=1145.19 (M+2H)²⁺

(calcd: 2287.91)

### Examples 2 to 6, 10, 11, 14 to 19, 24 to 28, and 31

By the same approach as in Example 1 mentioned above, the peptide of interest was synthesized, starting at the removal of the Fmoc group, in accordance with the general method described above using Sieber amide resin (Watanabe Chemical Industries, Ltd.). This synthesis was performed using Liberty Blue from CEM Corp. as a solid-phase synthesizer in accordance with manufacturer's manual, and each residue was introduced using DIC/Oxyma for elongation into the peptide chain of interest. For the introduction of each residue, the reaction was performed under conditions of the method 1-1, 1-2, 1-3, 1-4, or 1-5. Fmoc removal was carried out in accordance with conditions of the method 2-1 or 2-2.

After elongation into the peptide chain of interest, the chloroacetyl group was introduced. The introduction method is any of the methods 3-1, 3-2, and 3-3 described above.

The deprotection of a side chain and excision from the solid-phase resin were performed using the reactant cocktail of the method 4-1 or 4-2. The filtrate was added to an excess of diisopropyl ether or a mixed solvent of diisopropyl ether and hexane or diethyl ether and hexane cooled to 0° C. to obtain precipitates. The obtained solid was washed again with a small amount of diethyl ether or a mixed solvent of diisopropyl ether and hexane or diethyl ether and hexane cooled to 0° C., and then dried under reduced pressure.

The solid thus obtained was used in peptide cyclization reaction. For the cyclization reaction of the peptide, the peptide was dissolved in DMSO or a mixed solvent of DMSO/H₂O, MeCN/H₂O, DMSO/IPA/H₂O, or DMSO/MeCN/H₂O such that the final concentration of the peptide was 2 mM to 5 mM based on the number of moles of the solid-phase resin used. Then, 5 to 35 equivalents of triethylamine were added to the solution, and the mixture was shaken at room temperature overnight or for 2 hours. The obtained reaction solution was concentrated under reduced pressure.

The obtained residue was separated and purified under conditions described in Table 8 below, and freeze-dried to obtain the peptide of interest.

Compounds described in Table 8 below were obtained by the synthesis method described above. For the compounds of Examples 6, 14, 15, 19, 24, 25, and 28, the introduction of the 13th residue was carried out using HATU/DIEA. For the compounds of Examples 26 and 27, the introduction of the 13th residue and the 14th residue was carried out using HATU/DIEA.

The synthesized peptides, purification conditions, analysis conditions, and retention times, and found values in ESI-MS(+) were described in Table 8 below.

TABLE 8

| | | Purification | |
|---|---|---|---|
| | | condition | |
| | | Analysis | |
| | | condition | |
| Example | Sequence and structure of synthesized | (retention time | ESI-MS(+) |
| No. | peptide | (min)) | found (calcd) |
| 2 | ClAc-H2PyOMe-Y-S-W-MeA-QaEt-Y- W7C-S-Y-3Py56dOMe-MeG- W72pyrm-C-NH2 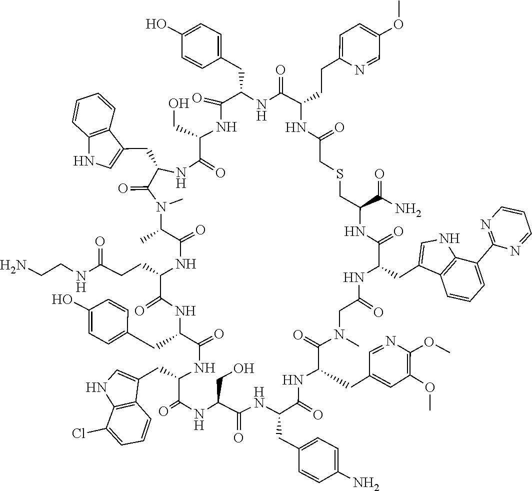 [SMILES: COc1ccc(CC[C@@H]2NC(=O)CSC[C@@H](C(N)=O)NC(=O)[C@H](Cc3c[nH]c4c(-c5ncccn5)cccc34)NC(=O)CN(C)C(=O)[C@H](Cc3cnc(OC)c(OC)c3)NC(=O)[C@H](Cc3ccc(N)cc3)NC(=O)[C@H](CO)NC(=O)[C@H](Cc3c[nH]c4c(Cl)cccc34)NC(=O)[C@H](Cc3ccc(O)cc3)NC(=O)[C@H](CCC(=O)NCCN)NC(=O)[C@H](C)N(C)C(=O)[C@H](Cc3c[nH]c4ccccc34)NC(=O)[C@H](CO)NC(=O)[C@H](Cc3ccc(O)cc3)NC2=O)nc1] | P-C Q-C (7.98) | (M + 2H)²⁺ = 1112.37 (2221.88) |
| 3 | ClAc-H2PyOMe-Y-S-W-MeA- Qpyrli3RMe-F4COO-W7C-S-Y- 3Py56dOMe-MeG-W72pyrm-C-NH2 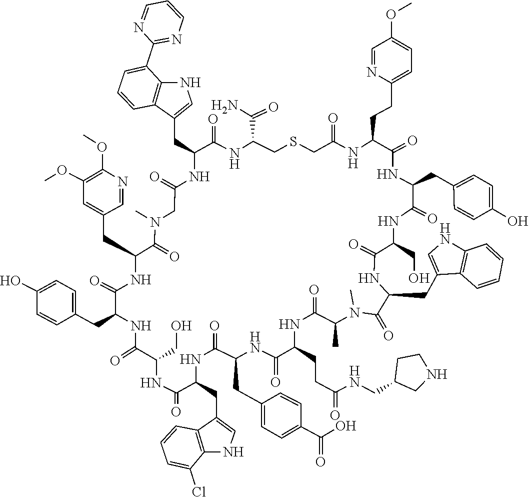 [SMILES: COc1ccc(CC[C@@H]2NC(=O)CSC[C@@H](C(N)=O)NC(=O)[C@H](Cc3c[nH]c4c(-c5ncccn5)cccc34)NC(=O)CN(C)C(=O)[C@H](Cc3cnc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(C(=O)O)cc3)NC(=O)[C@H](CCC(=O)NC[C@@H]3CCNC3)NC(=O)[C@H](C)N(C)C(=O)[C@H](Cc3c[nH]c4ccccc34)NC(=O)[C@H](CO)NC(=O)[C@H](Cc3ccc(O)cc3)NC2=O)nc1] | P-C Q-D (8.55) | (M + 2H)²⁺ = 1146.45 (2289.90) |
| 4 | ClAc-H2PyOMe-Y-S-W-MeA-QaPr- F4COO-W7C-S-Y-3Py56dOMe-MeG- W72pyrm-C-NH2 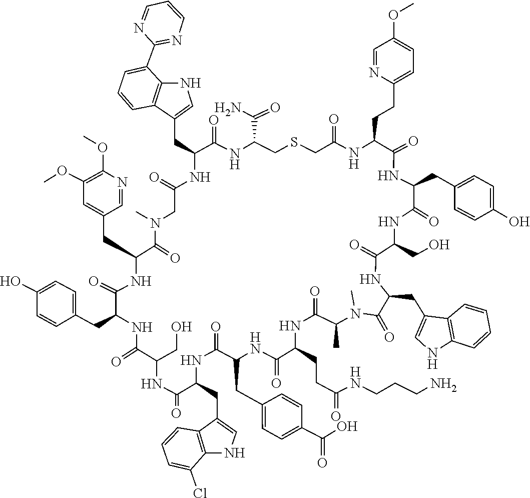 | P-C Q-D (8.46) | (M + 2H)²⁺ = 1133.44 (2263.89) |
| 5 | ClAc-H2PyOMe-Y-S-W-MeA-DapAc- Y-W7C-S-Y-3Py56dOMe-MeG- W72pyrm-C-NH2 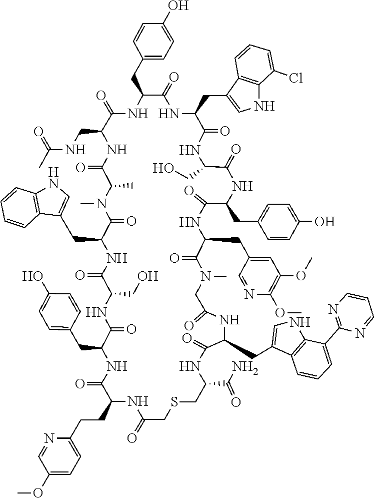 [SMILES: COc1ccc(CC[C@@H]2NC(=O)CSC[C@@H](C(N)=O)NC(=O)[C@H](Cc3c[nH]c4c(-c5ncccn5)cccc34)NC(=O)CN(C)C(=O)[C@H](Cc3cnc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(O)cc3)NC(=O)[C@H](CNC(C)=O)NC(=O)[C@H](C)N(C)C(=O)[C@H](Cc3c[nH]c4ccccc34)NC(=O)[C@H](CO)NC(=O)[C@H](Cc3ccc(O)cc3)NC2=O)nc1] | P-C Q-D (10.54) | (M + 2H)²⁺= 1090.91 (2178.84) |
| 6 | ClAc-H2PyOMe-Y-S-W-MeA- Qpyrli3RMe-F4COO-W7C-S-Y- 3Py56dOMe-MeG-W72Pyrm5F-C-NH2 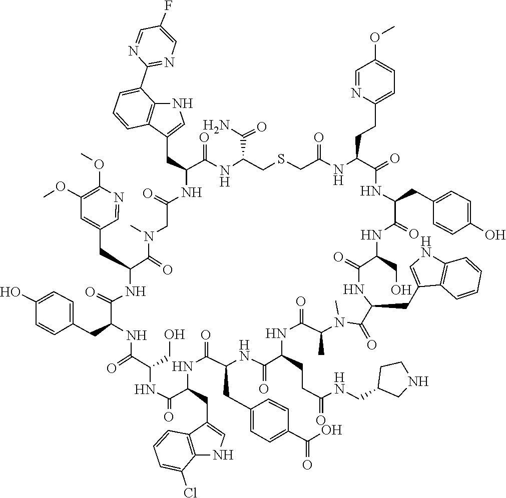 [SMILES: COc1ccc(CC[C@@H]2NC(=O)CSC[C@@H](C(N)=O)NC(=O)[C@H](Cc3c[nH]c4c(-c5ncc(F)cn5)cccc34)NC(=O)CN(C)C(=O)[C@H](Cc3cnc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(C(=O)O)cc3)NC(=O)[C@H](CCC(=O)NC[C@@H]3CCNC3)NC(=O)[C@H](C)N(C)C(=O)[C@H](Cc3c[nH]c4ccccc34)NC(=O)[C@H](CO)NC(=O)[C@H](Cc3ccc(O)cc3)NC2=O)nc1] | P-B Q-C (9.40) | (M + 2H)²⁺ = 1155.23 (2307.89) |
| 10 | ClAc-H2PyOMe-Y-S-W-CeG-Q-Y- W7C-S-Y-3Py56dOMe-MeG- W72pyrm-C-NH2 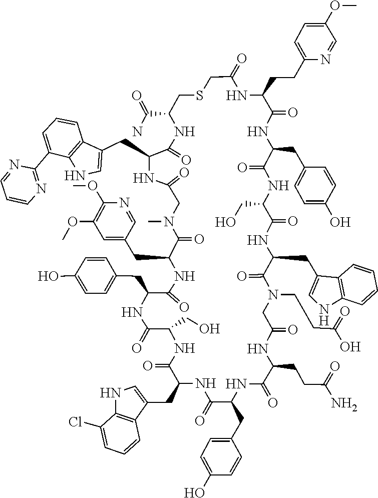 | P-C Q-D (10.34) | (M + 2H)²⁺ = 1112.67 (2222.83) |
| 11 | ClAc-H2PyOMe-Y-S-W-CeG- Qpyrli3RMe-Y-W7C-S-Y-3Py56dOMe- MeG-W72pyrm-C-NH2 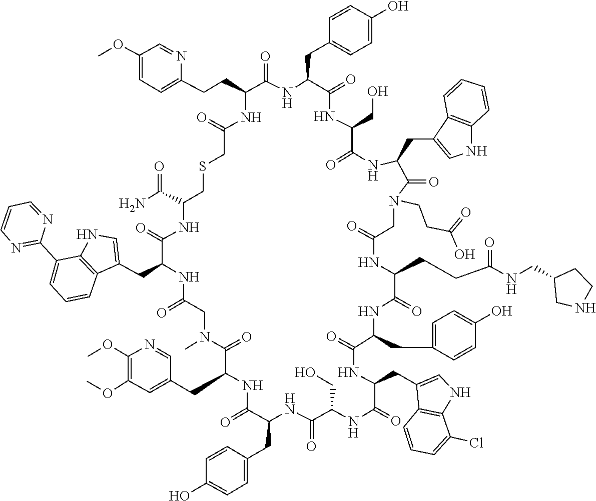 | P-B Q-C (7.28) | (M + 2H)²⁺ = 1154.45 (2305.90) |
| 14 | ClAc-H2PyOMe-Y-S-W-MeA- Qpyrli3RMe-F4COO-W7C-S-Y- 3Py56dOMe-MeG-W72Py-C-NH2 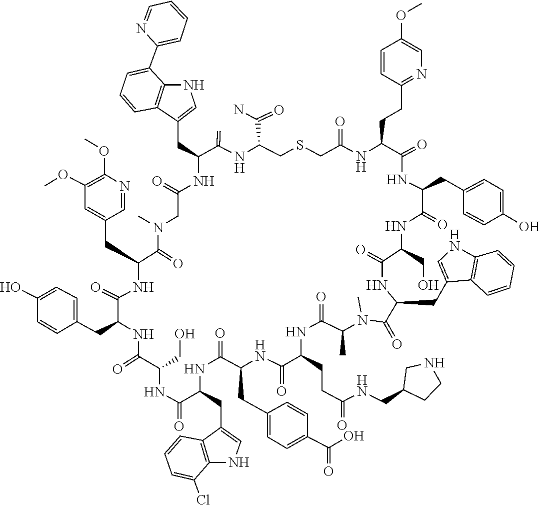 | P-C Q-C (9.08) | (M + 2H)²⁺ = 1145.91 (2288.91) |
| 15 | ClAc-H2PyOMe-Y-S-W-MeA- QaMe3Az-F4COO-W7C-S-Y- 3Py56dOMe-MeG-W72Py-C-NH2 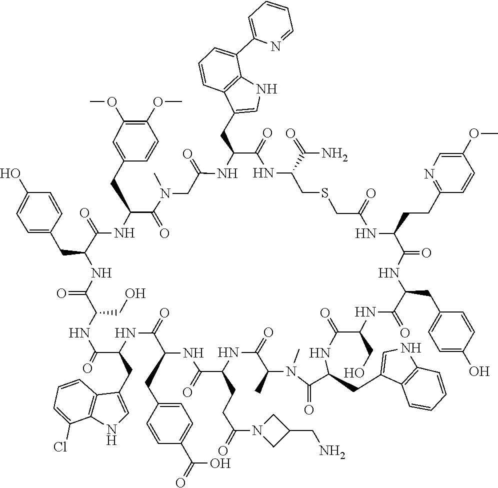 [SMILES: COc1ccc(CC[C@@H]2NC(=O)CSC[C@@H](C(N)=O)NC(=O)[C@H](Cc3c[nH]c4c(-c5ccccn5)cccc34)NC(=O)CN(C)C(=O)[C@H](Cc3ccc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(C(=O)O)cc3)NC(=O)[C@H](CCC(=O)N3CC(CN)C3)NC(=O)[C@H](C)N(C)C(=O)[C@H](Cc3c[nH]c4ccccc34)NC(=O)[C@H](CO)NC(=O)[C@H](Cc3ccc(O)cc3)NC2=O)nc1] | P-C Q-C (8.91) | (M + 2H)²⁺ = 1138.93 (2274.89) |
| 16 | ClAc-H2PyOMe-Y-S-W-MeA- QaMe3Az-Y-W7C-S-Y-3Py56dOMe- MeG-W73Pyrd-C-NH2 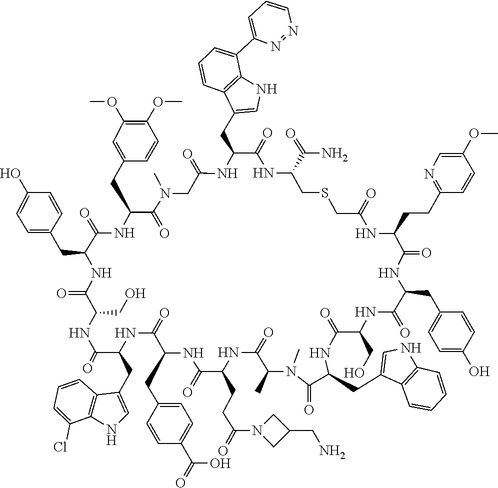 [SMILES: COc1ccc(CC[C@@H]2NC(=O)CSC[C@@H](C(N)=O)NC(=O)[C@H](Cc3c[nH]c4c(-c5cccnn5)cccc34)NC(=O)CN(C)C(=O)[C@H](Cc3ccc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(C(=O)O)cc3)NC(=O)[C@H](CCC(=O)N3CC(CN)C3)NC(=O)[C@H](C)N(C)C(=O)[C@H](Cc3c[nH]c4ccccc34)NC(=O)[C@H](CO)NC(=O)[C@H](Cc3ccc(O)cc3)NC2=O)nc1] | P-C Q-C (7.10) | (M + 2H)²⁺ = 1125.48 (2247.89) |
| 17 | ClAc-H2PyOMe-Y-S-W-MeA-DapAc- F4COO-W7C-S-Y-3Py56dOMe-MeG- W73Pyrd-C-NH2 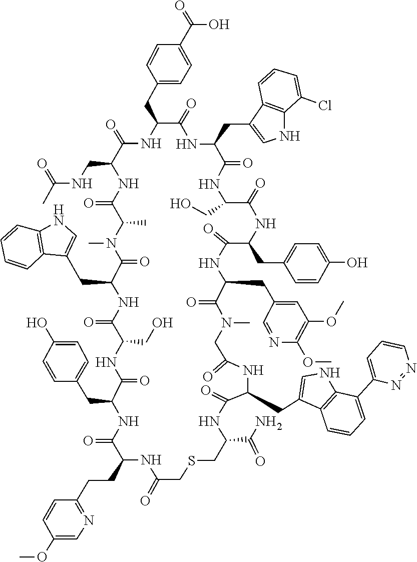 [SMILES: COc1ccc(CC[C@@H]2NC(=O)CSC[C@@H](C(N)=O)NC(=O)[C@H](Cc3c[nH]c4c(-c5cccnn5)cccc34)NC(=O)CN(C)C(=O)[C@H](Cc3cnc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(C(=O)O)cc3)NC(=O)[C@H](CNC(C)=O)NC(=O)[C@H](C)N(C)C(=O)[C@H](Cc3c[nH]c4ccccc34)NC(=O)[C@H](CO)NC(=O)[C@H](Cc3ccc(O)cc3)NC2=O)nc1] | P-C Q-C (9.56) | (M + 2H)²⁺ = 1104.84 (2206.83) |
| 18 | ClAc-H2PyOMe-Y-S-W-MeA-DapAc- Y-W7C-S-Y-3Py56dOMe-MeG- W73Pyrd-C-NH2 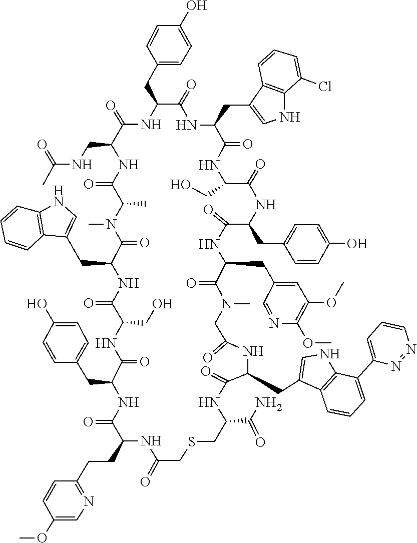 [SMILES: COc1ccc(CC[C@@H]2NC(=O)CSC[C@@H](C(N)=O)NC(=O)[C@H](Cc3c[nH]c4c(-c5cccnn5)cccc34)NC(=O)CN(C)C(=O)[C@H](Cc3cnc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(O)cc3)NC(=O)[C@H](CNC(C)=O)NC(=O)[C@H](C)N(C)C(=O)[C@H](Cc3c[nH]c4ccccc34)NC(=O)[C@H](CO)NC(=O)[C@H](Cc3ccc(O)cc3)NC2=O)nc1] | P-C Q-C (9.52) | (M + 2H)²⁺ = 1090.87 (2178.84) |
| 19 | ClAc-H2PyOMe-Y-S-W-MeA- QaMe3Az-F4COO-W7C-S-Y- F34dOMe-MeG-W72Py5COO-C-NH2 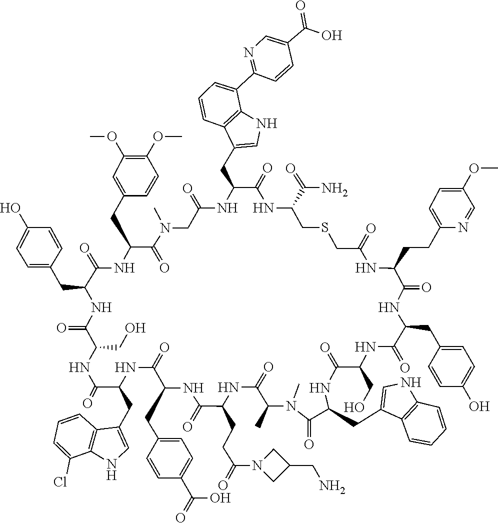 [SMILES: COc1ccc(CC[C@@H]2NC(=O)CSC[C@@H](C(N)=O)NC(=O)[C@H](Cc3c[nH]c4c(-c5ccc(C(=O)O)cn5)cccc34)NC(=O)CN(C)C(=O)[C@H](Cc3ccc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(C(=O)O)cc3)NC(=O)[C@H](CCC(=O)N3CC(CN)C3)NC(=O)[C@H](C)N(C)C(=O)[C@H](Cc3c[nH]c4ccccc34)NC(=O)[C@H](CO)NC(=O)[C@H](Cc3ccc(O)cc3)NC2=O)nc1] | P-C Q-D (10.79) | (M + 2H)²⁺ = 1160.18 (2317.89) |
| 24 | ClAc-H2PyOMe-Y-S-W-MeA-QaPr- F4COO-W7C-S-Y-3Py56dOMe-MeG- W73PyzlMe-C-NH2 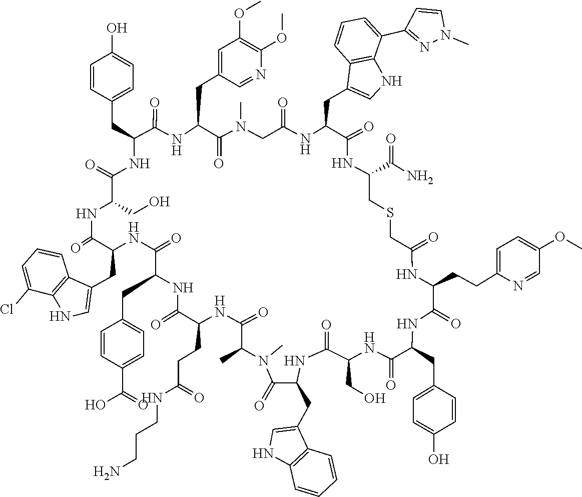 [SMILES: COc1ccc(CC[C@@H]2NC(=O)CSC[C@@H](C(N)=O)NC(=O)[C@H](Cc3c[nH]c4c(-c5ccn(C)n5)cccc34)NC(=O)CN(C)C(=O)[C@H](Cc3cnc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(C(=O)O)cc3)NC(=O)[C@H](CCC(=O)NCCCN)NC(=O)[C@H](C)N(C)C(=O)[C@H](Cc3c[nH]c4ccccc34)NC(=O)[C@H](CO)NC(=O)[C@H](Cc3ccc(O)cc3)NC2=O)nc1] | P-B Q-B (3.18) | (M + 2H)²⁺ = 1134.30 (2265.90) |
| 25 | ClAc-H2PyOMe-Y-S-W-MeA-DapAc- Y-W7C-S-Y-3Py56dOMe-MeG- W72Py5COO-C-NH2 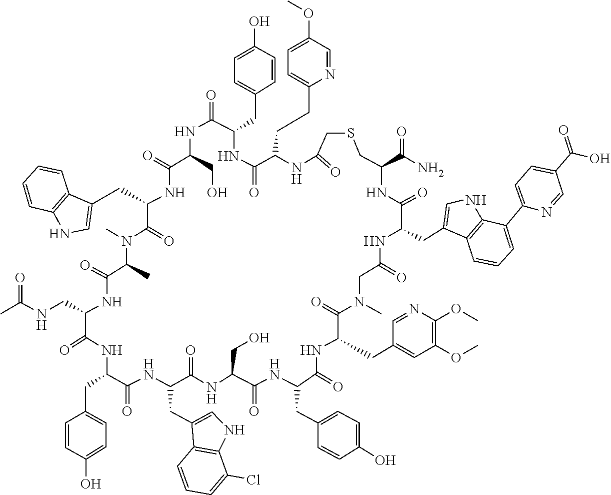 | P-C Q-B (3.84) | (M + 2H)²⁺ = 1112.37 (2221.83) |
| 26 | ClAc-H2PyOMe-Y-S-W-MeA- Qpyrli3RMe-Y-W7C-S-Y-3Py56dOMe- MeG-W72Py5CON-C-NH2 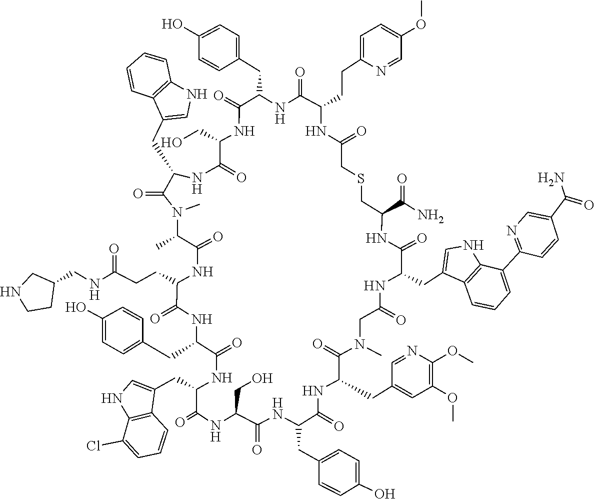 | P-A Q-B (2.85) | (M + 2H)²⁺ = 1153.21 (2303.92) |
| 27 | ClAc-H2PyOMe-Y-S-W-MeA- QaMe3Az-F4COO-W7C-S-Y- F34dOMe-MeG-W72Py5CON-C-NH2 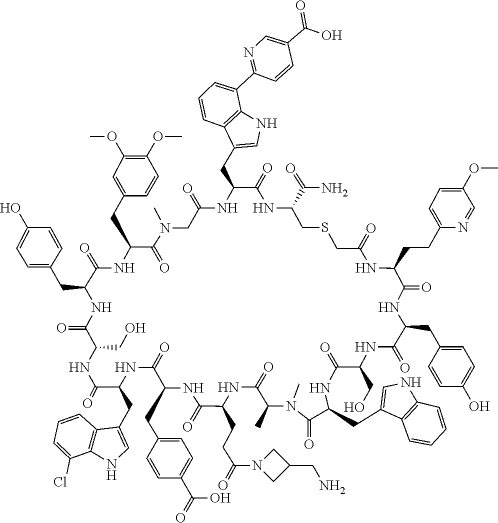 [SMILES: COc1ccc(CC[C@@H]2NC(=O)CSC[C@@H](C(N)=O)NC(=O)[C@H](Cc3c[nH]c4c(-c5ccc(C(=O)O)cn5)cccc34)NC(=O)CN(C)C(=O)[C@H](Cc3ccc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(C(=O)O)cc3)NC(=O)[C@H](CCC(=O)N3CC(CN)C3)NC(=O)[C@H](C)N(C)C(=O)[C@H](Cc3c[nH]c4ccccc34)NC(=O)[C@H](CO)NC(=O)[C@H](Cc3ccc(O)cc3)NC2=O)nc1] | P-C Q-B (3.07) | (M + 2H)²⁺ = 1159.94 (2316.90) |
| 28 | ClAc-H2PyOMe-Y-S-W-MeA- QaMe3Az-F4COO-W7C-S-Y- 3Py56dOMe-MeG-W73Pyrd-C-NH2 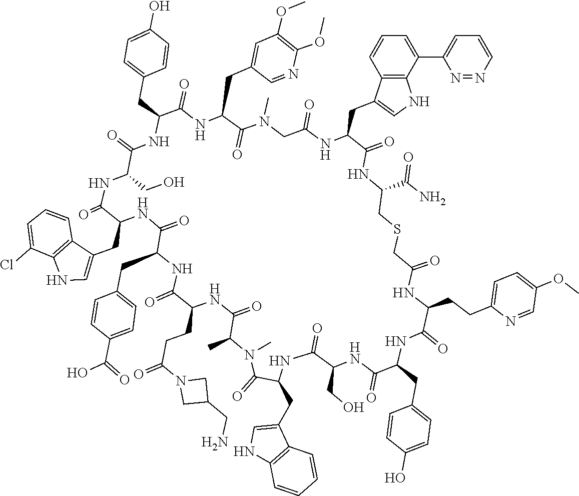 [SMILES: COc1ccc(CC[C@@H]2NC(=O)CSC[C@@H](C(N)=O)NC(=O)[C@H](Cc3c[nH]c4c(-c5cccnn5)cccc34)NC(=O)CN(C)C(=O)[C@H](Cc3cnc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(C(=O)O)cc3)NC(=O)[C@H](CCC(=O)N3CC(CN)C3)NC(=O)[C@H](C)N(C)C(=O)[C@H](Cc3c[nH]c4ccccc34)NC(=O)[C@H](CO)NC(=O)[C@H](Cc3ccc(O)cc3)NC2=O)nc1] | P-B Q-A (5.50) | (M + 2H)²⁺ = 1139.45 (2275.89) |
| 31 | ClAc-H2PyOMe-Y-S-W-CeG-Q- F4COO-W7C-S-Y-3Py56dOMe-MeG- W72pyrm-C-NH2 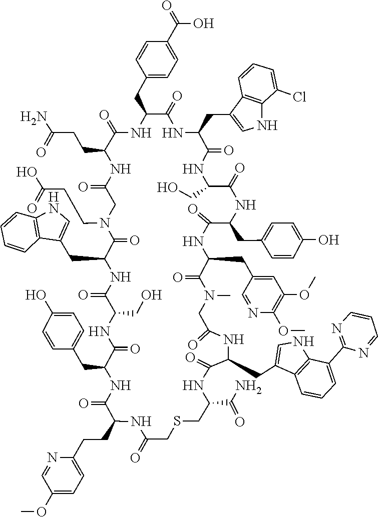 [SMILES: COc1ccc(CC[C@@H]2NC(=O)CSC[C@@H](C(N)=O)NC(=O)[C@H](Cc3c[nH]c4c(-c5ncccn5)cccc34)NC(=O)CN(C)C(=O)[C@H](Cc3cnc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(C(=O)O)cc3)NC(=O)[C@H](CCC(N)=O)NC(=O)CN(CCC(=O)O)C(=O)[C@H](Cc3c[nH]c4ccccc34)NC(=O)[C@H](CO)NC(=O)[C@H](Cc3ccc(O)cc3)NC2=O)nc1] | P-C Q-D (10.43) | (M + 2H)²⁺ = 1126.64 (2250.82) |

### Synthesis of Compound According to Example 7 Sequence: ClAc—H2PyOMe-Y—S—W—CeG-DapAc—Y—W7C—S—Y-3Py56dOMe-MeG-W72pyrm-C—NH2

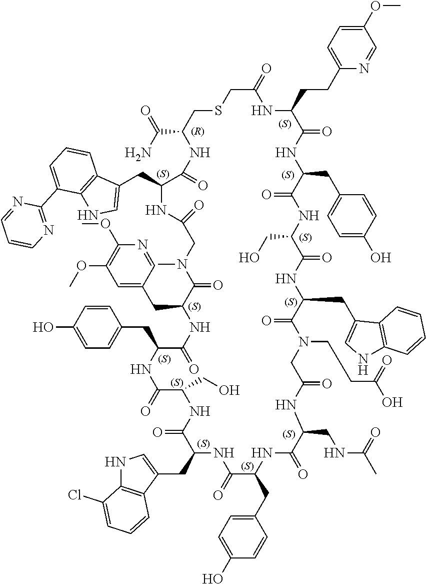

Elongation into the peptide chain of interest was performed in accordance with the general method described above. Specifically, the peptide of interest was synthesized, starting at the removal of the Fmoc group, by the general method mentioned above using Sieber amide resin (Watanabe Chemical Industries, Ltd., 0.47 mmol/g, 0.21 g). This synthesis was performed using Syro I from Biotage Japan Ltd. as a solid-phase synthesizer in accordance with manufacturer's manual. Each residue was introduced using Fmoc-AA/HATU/DIEA (6.3 equivalents/6 equivalents/12.6 equivalents) per equivalent of the resin, and reaction was performed under conditions of the method 1-1 or 1-3. Specifically, the reaction was basically performed twice at 75° C. for 20 minutes in DMF under microwave irradiation. However, the reaction was performed twice at room temperature for 30 minutes for the 14th residue. The reaction was performed once at room temperature for 120 minutes in DMF using Fmoc-AA/DIPCI/Oxyma pure (6.3 equivalents/12.6 equivalents/6 equivalents) for the first residue, the 11th residue, and the 13th residue. Fmoc was removed through reaction with a 20% solution of piperidine in DMF at room temperature for 5 minutes and subsequent reaction for 15 minutes in accordance with the method 2-2. The chloroacetyl group was introduced by, after removal of the Fmoc group of an α-amino group by the method mentioned above, adding chloroacetic acid (approximately 6.3 equivalents), HCTU (approximately 6 equivalents), and DIEA (approximately 6.3 equivalents) to the solid-phase resin carrying the Fmoc-protected peptide obtained in the preceding step in accordance with the method 3-4, and shaking the mixture at room temperature for 30 minutes in DMF. For the deprotection of a side chain and excision from the solid-phase resin, first, the resin obtained after the chloroacetyl group introduction step was washed with DMF and DCM, then washed with diethyl ether, and then dried under reduced pressure. Subsequently, the reactant cocktail (4 mL) of the method 4-1 was added to a reaction container containing the solid-phase resin, and shaken at room temperature for 90 minutes. The reaction solution was collected by filtration from the frit. The solid-phase resin remaining in the reaction container was shaken again with the cocktail for excision, and a solution component was collected from the frit and mixed with the filtrate mentioned above. This filtrate was added to an excess of a diethyl ether/hexane (1/1) mixed solvent cooled to 0° C. so that cloudy precipitation occurred. This mixture was centrifuged (9000 rpm, 2 min), and the solution was decanted. The obtained solid was washed again with a small amount of a diethyl ether/hexane (1/1) mixed solvent cooled to 0° C., and then dried under reduced pressure. The obtained solid was used in subsequent cyclization reaction. For the cyclization reaction of the peptide, the peptide was dissolved in a DMSO/MeCN/H₂O (18/1/1) mixed solvent such that the final concentration of the peptide was 2 mM based on the number of moles of the solid-phase resin used. Then, 10 equivalents of triethylamine were added to the solution, and the mixture was stirred overnight at room temperature. Then, the reaction solution was concentrated under reduced pressure.

The obtained reaction mixture was purified under the following conditions (column: Waters Xbridge (R) C18 5 μm 50× 150 mm; mobile phase: A=0.1% TFA in H₂O, B=0.1% TFA in MeCN; temperature: 40° C.; gradient (% B): 5-26% over 3 minutes, subsequently 26-31% over 8 minutes, and subsequently 31-60% over 1 minute; flow rate: 120 mL/min).

The purity of the compound of interest was calculated from an area ratio of an LC/MS (UV wavelength: 225 nm) chromatogram obtained under the following analysis condition and was consequently 97.4%.

Analysis condition Q-C: retention time=10.21 min

ESI-MS(+) found m/z=1112.62 (M+2H)²⁺

(calcd: 2222.83)

### Examples 8, 9, 13, 20 to 23, and 30

By the same approach as in Example 7 mentioned above, the peptide of interest was synthesized, starting at the removal of the Fmoc group, in accordance with the general method described above using Sieber amide resin (Watanabe Chemical Industries, Ltd.). This synthesis was performed using Syro I from Biotage Japan Ltd. as a solid-phase synthesizer in accordance with manufacturer's manual. Each residue was introduced using HATU/DIEA for elongation into the peptide chain of interest. For the introduction of each residue, the reaction was performed under conditions of the method 1-1 or 1-3. Fmoc removal was carried out in accordance with conditions of the method 2-2 or 2-7.

After elongation into the peptide chain of interest, the chloroacetyl group was introduced. The introduction method is any of the methods 3-2, 3-3, and 3-4 described above.

The deprotection of a side chain and excision from the solid-phase resin were performed using the reactant cocktail of the method 4-1. The filtrate was added to an excess of diethyl ether, diisopropyl ether, or a mixed solvent of diethyl ether and hexane cooled to 0° C. to obtain precipitates. The obtained solid was washed again with a small amount of diethyl ether, diisopropyl ether, or a mixed solvent of diethyl ether and hexane cooled to 0° C., and then dried under reduced pressure.

The solid thus obtained was used in peptide cyclization reaction. For the cyclization reaction of the peptide, the peptide was dissolved in DMSO or a mixed solvent of DMSO/H₂O or DMSO/IPA/H₂O such that the final concentration of the peptide was 2 mM to 5 mM based on the number of moles of the solid-phase resin used. Then, 5 to 10 equivalents of triethylamine were added to the solution, and the mixture was shaken at room temperature overnight or for 1 hour. The obtained reaction solution was concentrated under reduced pressure.

The obtained residue was separated and purified under conditions described in Table 9 below, and freeze-dried to obtain the peptide of interest.

Compounds described in Table 9 below were obtained by the synthesis method described above. For the compounds of Examples 8, 9, and 30, the introduction of the first residue, the 11th residue, and the 13th residue was carried out using DIC/Oxyma pure. The synthesized peptides, purification conditions, analysis conditions, and retention times, and found values in ESI-MS(+) were described in Table 9 below.

TABLE 9

| | | Purification | |
|---|---|---|---|
| | | condition | |
| | | Analysis | |
| | | condition | |
| Example | Sequence and structure of synthesized | (retention time | ESI-MS(+) |
| No. | peptide | (min)) | found (calcd) |
| 8 | ClAc-H2PyOMe-Y-S-W-MeA-Q-Y- W7C-S-Y-3Py56dOMe-MeG- W72pyrm-C-NH2 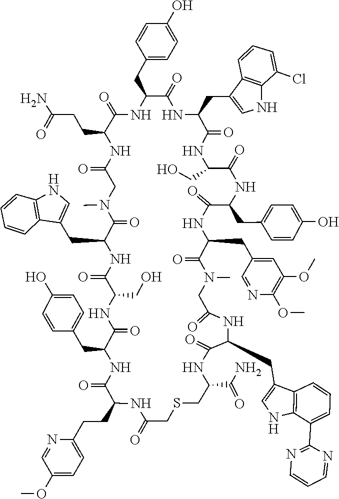 [SMILES: COc1ccc(CC[C@@H]2NC(=O)CSC[C@@H](C(N)=O)NC(=O)[C@H](Cc3c[nH]c4c(-c5ncccn5)cccc34)NC(=O)CN(C)C(=O)[C@H](Cc3cnc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(O)cc3)NC(=O)[C@H](CCC(N)=O)NC(=O)CN(C)C(=O)[C@H](Cc3c[nH]c4ccccc34)NC(=O)[C@H](CO)NC(=O)[C@H](Cc3ccc(O)cc3)NC2=O)nc1] | P-C Q-D (9.89) | (M + 2H)²⁺ = 1090.66 (2178.84) |
| 9 | ClAc-H2PyOMe-Y-S-W-MeA-Q- F4COO-W7C-S-Y-3Py56dOMe-MeG- W72pyrm-C-NH2 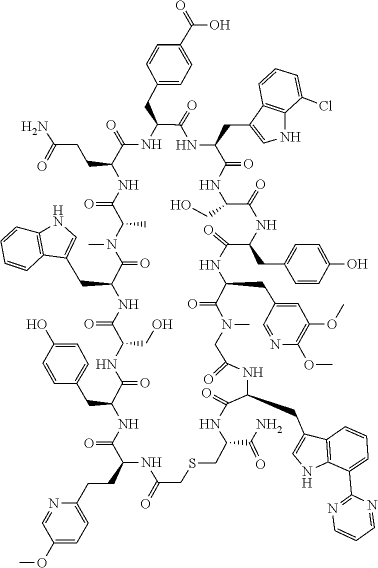 [SMILES: COc1ccc(CC[C@@H]2NC(=O)CSC[C@@H](C(N)=O)NC(=O)[C@H](Cc3c[nH]c4c(-c5ncccn5)cccc34)NC(=O)CN(C)C(=O)[C@H](Cc3cnc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(C(=O)O)cc3)NC(=O)[C@H](CCC(N)=O)NC(=O)[C@H](C)N(C)C(=O)[C@H](Cc3c[nH]c4ccccc34)NC(=O)[C@H](CO)NC(=O)[C@H](Cc3ccc(O)cc3)NC2=O)nc1] | P-C Q-D (9.93) | (M + 2H)²⁺ = 1104.58 (2206.83) |
| 13 | ClAc-H2PyOMe-Y-S-W-MeD-DapAc- Y-W7C-S-Y-3Py56dOMe-MeG- W72pyrm-C-NH2 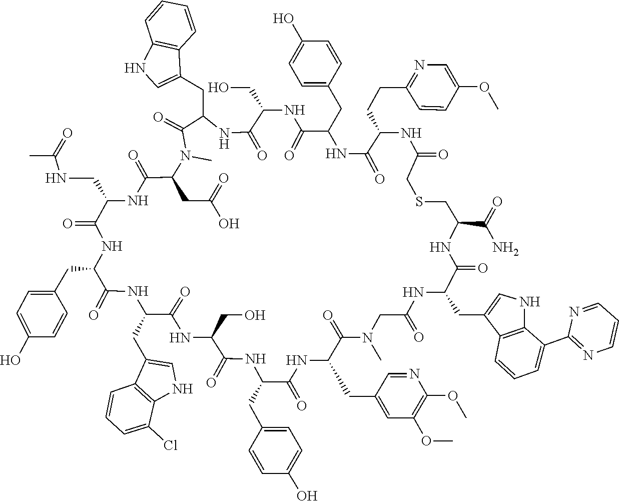 [SMILES: COc1ccc(CC[C@@H]2NC(=O)CSC[C@@H](C(N)=O)NC(=O)[C@H](Cc3c[nH]c4c(-c5ncccn5)cccc34)NC(=O)CN(C)C(=O)[C@H](Cc3cnc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(O)cc3)NC(=O)[C@H](CNC(C)=O)NC(=O)[C@H](CC(=O)O)N(C)C(=O)C(Cc3c[nH]c4ccccc34)NC(=O)[C@H](CO)NC(=O)C(Cc3ccc(O)cc3)NC2=O)nc1] | P-B Q-C (9.62) | (M + 2H)²⁺ = 1112.84 (2222.83) |
| 20 | ClAc-H2PyOMe-Y-S-W-MeA- QaMe3Az-F4COO-W7C-S-Y- F34dOMe-MeG-W72pyrm-C-NH2 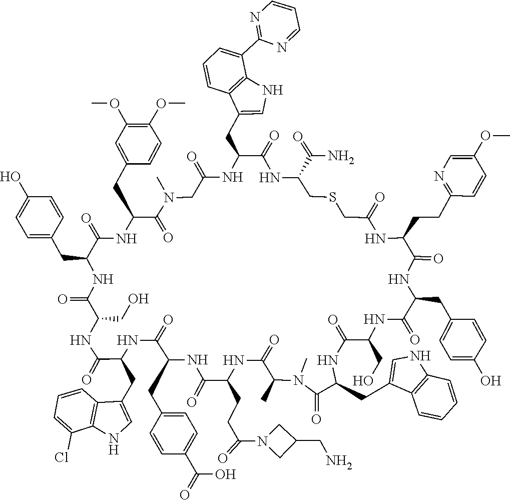 [SMILES: COc1ccc(CC[C@@H]2NC(=O)CSC[C@@H](C(N)=O)NC(=O)[C@H](Cc3c[nH]c4c(-c5ncccn5)cccc34)NC(=O)CN(C)C(=O)[C@H](Cc3ccc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(C(=O)O)cc3)NC(=O)[C@H](CCC(=O)N3CC(CN)C3)NC(=O)[C@H](C)N(C)C(=O)[C@H](Cc3c[nH]c4ccccc34)NC(=O)[C@H](CO)NC(=O)[C@H](Cc3ccc(O)cc3)NC2=O)nc1] | P-C Q-C (8.90) | (M + 2H)²⁺ = 1138.86 (2274.89) |
| 21 | ClAc-H2PyOMe-Y-S-W-CeG- QaMe3Az-F4COO-W7C-S-Y- F34dOMe-MeG-W72pyrm-C-NH2 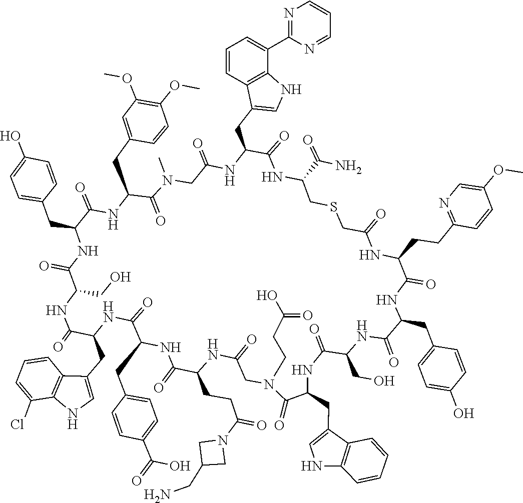 [SMILES: COc1ccc(CC[C@@H]2NC(=O)CSC[C@@H](C(N)=O)NC(=O)[C@H](Cc3c[nH]c4c(-c5ncccn5)cccc34)NC(=O)CN(C)C(=O)[C@H](Cc3ccc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(C(=O)O)cc3)NC(=O)[C@H](CCC(=O)N3CC(CN)C3)NC(=O)CN(CCC(=O)O)C(=O)[C@H](Cc3c[nH]c4ccccc34)NC(=O)[C@H](CO)NC(=O)[C@H](Cc3ccc(O)cc3)NC2=O)nc1] | P-C Q-C (8.07) | (M + 2H)²⁺ = 1160.72 (2318.88) |
| 22 | ClAc-H3Pyrd60Me-Y-S-W-MeA- QaMe3Az-F4COO-W7C-S-Y- 3Py56dOMe-MeG-W72pyrm-C-NH2 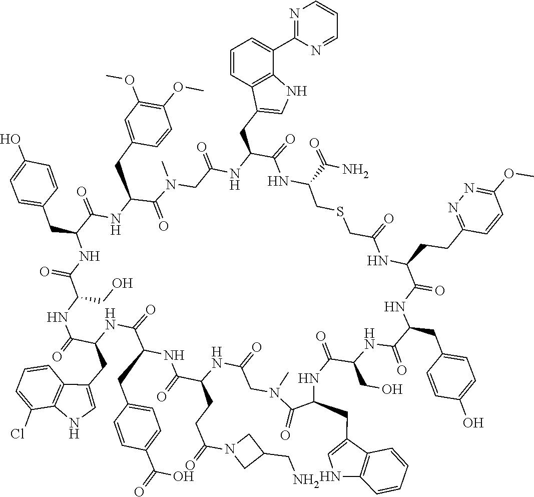 [SMILES: COc1ccc(CC[C@@H]2NC(=O)CSC[C@@H](C(N)=O)NC(=O)[C@H](Cc3c[nH]c4c(-c5ncccn5)cccc34)NC(=O)CN(C)C(=O)[C@H](Cc3ccc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(C(=O)O)cc3)NC(=O)[C@H](CCC(=O)N3CC(CN)C3)NC(=O)CN(C)C(=O)[C@H](Cc3c[nH]c4ccccc34)NC(=O)[C@H](CO)NC(=O)[C@H](Cc3ccc(O)cc3)NC2=O)nn1] | P-B Q-B (3.57) | (M + 2H)²⁺ = 1139.84 (2276.88) |
| 23 | ClAc-H2PyOMe-Y-S-W-MeE-QaPr-Y- W7C-S-Y-3Py56dOMe-MeG- W72pyrm-C-NH2 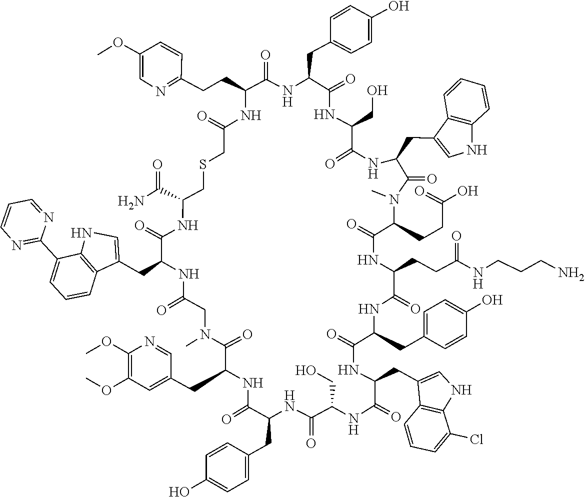 [SMILES: COc1ccc(CC[C@@H]2NC(=O)CSC[C@@H](C(N)=O)NC(=O)[C@H](Cc3c[nH]c4c(-c5ncccn5)cccc34)NC(=O)CN(C)C(=O)[C@H](Cc3cnc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(O)cc3)NC(=O)[C@H](CCC(=O)NCCCN)NC(=O)[C@H](CCC(=O)O)N(C)C(=O)[C@H](Cc3c[nH]c4ccccc34)NC(=O)[C@H](CO)NC(=O)[C@H](Cc3ccc(O)cc3)NC2=O)nc1] | P-B Q-B (2.76) | (M + 2H)²⁺ = 1148.22 (2293.90) |
| 30 | ClAc-H2PyOMe-Y-S-W-CeG-DapAc- F4COO-W7C-S-Y-3Py56dOMe-MeG- W72pyrm-C-NH2 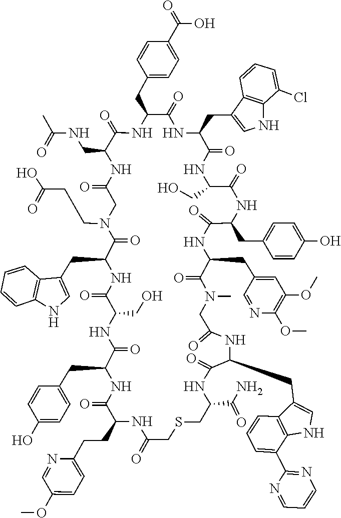 [SMILES: COc1ccc(CC[C@@H]2NC(=O)CSC[C@@H](C(N)=O)NC(=O)[C@H](Cc3c[nH]c4c(-c5ncccn5)cccc34)NC(=O)CN(C)C(=O)[C@H](Cc3cnc(OC)c(OC)c3)NC(=O)[C@H](Cc3ccc(O)cc3)NC(=O)[C@H](CO)NC(=O)[C@H](Cc3c[nH]c4c(Cl)cccc34)NC(=O)[C@H](Cc3ccc(C(=O)O)cc3)NC(=O)[C@H](CNC(C)=O)NC(=O)CN(CCC(=O)O)C(=O)[C@H](Cc3c[nH]c4ccccc34)NC(=O)[C@H](CO)NC(=O)[C@H](Cc3ccc(O)cc3)NC2=O)nc1] | P-C Q-C (10.17) | (M + 2H)²⁺ = 1126.62 (2250.82) |

### Synthesis of Compound According to Example 12 Sequence: ClAc—H2PyOMe-Y—S—W-MeA-Q4Pye-F4COO—W7C—S—Y-3Py56dOMe-MeG-W72pyrm-C—NH2

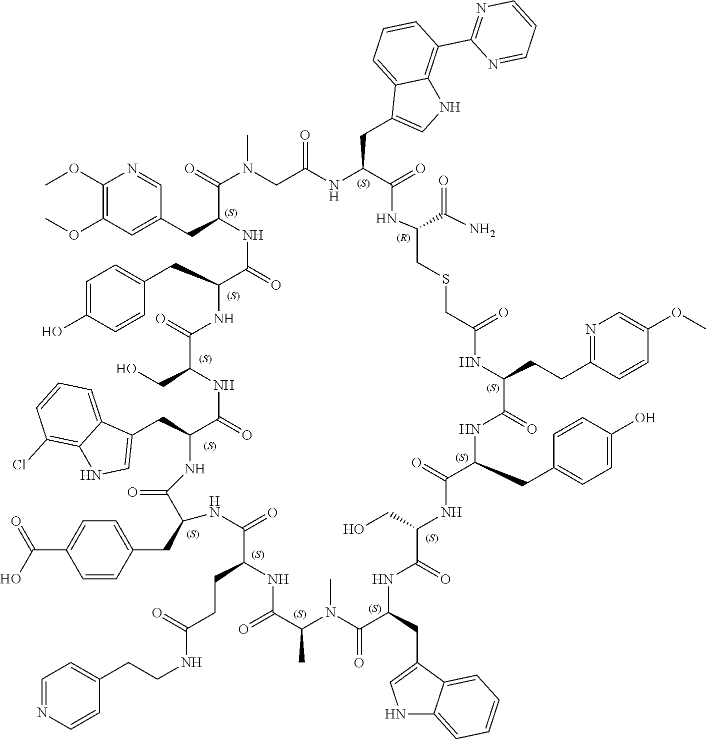

Elongation into the peptide chain of interest was performed in accordance with the general method described above. Specifically, the peptide of interest was synthesized, starting at the removal of the Fmoc group, by the general method mentioned above using Sieber amide resin (Watanabe Chemical Industries, Ltd., 0.47 mmol/g, 0.21 g). This synthesis was performed using Liberty Blue from CEM Corp. as a solid-phase synthesizer in accordance with manufacturer's manual. Each residue was introduced using Fmoc-AA/DIPCI/Oxyma pure (5.25 equivalents/10 equivalents/5 equivalents) per equivalent of the resin, and reaction was performed under conditions of the method 1-2, 1-3, or 1-4. Specifically, the reaction was basically performed once at 90° C. for 3 minutes in DMF under microwave irradiation. However, the reaction was performed once at 40° C. for 30 minutes for the first residue and the 11th residue. The reaction was performed once at 75° C. for 10 minutes for the 13th residue. The reaction was performed once at 50° C. for 15 minutes for the 14th residue. Fmoc was removed through reaction with a 20% solution of piperidine in DMF at 75° C. for 3 minutes in accordance with the method 2-1 or 2-2. However, the Fmoc removal for the first residue, the fourth residue, and the 11th residue was performed through reaction at room temperature for 5 minutes and subsequent reaction for 5 minutes. After elongation into the peptide chain of interest, allyl on the side chain of the sixth residue was removed through reaction at room temperature for 120 minutes with a catalytic amount of palladium (II) acetate, 1 equivalent of triphenylphosphine, and 10 equivalents of phenylsilane in DCM. Subsequently, the resin obtained after the preceding step was washed with DCM and then reacted at room temperature for 1 hour using 4-(2-aminoethyl)pyridine/HATU/DIEA (5 equivalents/5 equivalents/10 equivalents) in DMF. The chloroacetyl group was introduced by, after removal of the Fmoc group of an α-amino group by the method mentioned above, adding chloroacetic acid (approximately 10 equivalents), DIC (approximately 10 equivalents), and HOSu (approximately 10 equivalents) to the solid-phase resin carrying the Fmoc-protected peptide obtained in the preceding step in accordance with the method 3-1, and shaking the mixture at room temperature for 60 minutes in DMF. For the deprotection of a side chain and excision from the solid-phase resin, first, the resin obtained after the chloroacetyl group introduction step was washed with DMF and DCM, then washed with diethyl ether, and then dried under reduced pressure. Subsequently, the reactant cocktail (6 mL) of the method 4-1 was added to a reaction container containing the solid-phase resin, and shaken at room temperature for 90 minutes. The reaction solution was collected by filtration from the frit. The solid-phase resin remaining in the reaction container was shaken again with the cocktail for excision, and a solution component was collected from the frit and mixed with the filtrate mentioned above. This filtrate was added to an excess of a diethyl ether/hexane (1/1) mixed solvent cooled to 0° C. so that cloudy precipitation occurred. This mixture was centrifuged (9000 rpm, 2 min), and the solution was decanted. The obtained solid was washed again with a small amount of diethyl ether cooled to 0° C., and then dried under reduced pressure. The obtained solid was used in subsequent cyclization reaction. For the cyclization reaction of the peptide, the peptide was dissolved in a DMSO/IPA/H2O (90/5/5) mixed solvent such that the final concentration of the peptide was 5 mM based on the number of moles of the solid-phase resin used. Then, 5 equivalents of triethylamine were added to the solution, and the mixture was stirred at room temperature for 12 hours and then concentrated under reduced pressure.

The obtained crude product was purified under the following conditions (column: Waters Xbridge (R) C18 5 μm 30×150 mm; mobile phase: A=0.1% TFA in H₂O, B=0.1% TFA in MeCN; temperature: 40° C.; gradient (% B): 5-26% over 3 minutes, subsequently 26-31% over 8 minutes, and subsequently 31-60% over 1 minute; flow rate: 45 mL/min).

The purity of the compound of interest was calculated from an area ratio of an LC/MS (UV wavelength: 225 nm) chromatogram obtained under the following analysis condition and was consequently 93.6%.

Analysis condition Q-C: retention time=9.09 min

ESI-MS(+) found m/z=1157.22 (M+2H)²⁺ (calcd: 2311.89)

Synthesis of Compound According to Example 29 Sequence: ClAc—H2PyOMe-Y—S—W-MeK (cC12COO-isoE-isoE-)-QaPr—F4COO—W7C—S—Y-3Py56dOMe-MeG-W72pyrm-C—NH2

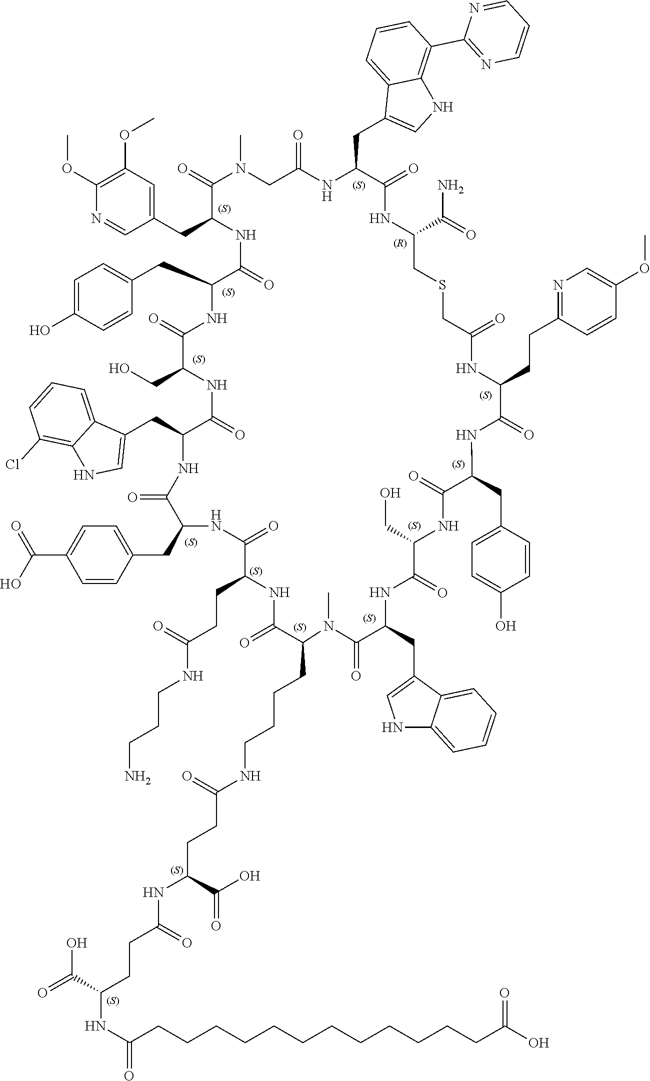

Elongation into the peptide chain of interest was performed in accordance with the general method described above. This synthesis was performed using Liberty Blue from CEM Corp. as a solid-phase synthesizer in accordance with manufacturer's manual. Specifically, a peptide was elongated from C at the 14th residue to MeK at the fifth residue (amino acid used: Alloc-MeK (Fmoc)-OH), starting at the removal of the Fmoc group, by the general method mentioned above using Sieber amide resin (Watanabe Chemical Industries, Ltd., 0.6 mmol/g, 0.44 g). Each residue was introduced using Fmoc-AA/DIPCI/Oxyma pure (4.2 equivalents/8 equivalents/4 equivalents) per equivalent of the resin, and reaction was performed under conditions of the method 1-2 or 1-4. Specifically, the reaction was basically performed once at 90° C. for 3 minutes in DMF under microwave irradiation. However, the reaction was performed once at 40° C. for 30 minutes for the sixth residue and the 13th residue. The reaction was performed once at 90° C. for 10 minutes for the 10th residue. The reaction was performed twice at 40° C. for 30 minutes for the 11th residue. The reaction was performed once at 50° C. for 15 minutes for the 14th residue. Fmoc was removed through reaction with a 10% solution of pyrrolidine in DMF at 90° C. for 1 minute in accordance with the method 2-4. However, the reaction for the 11th residue was performed twice at room temperature for 1 minutes in accordance with the method 2-5. Subsequently, Fmoc was removed from the fifth residue through reaction with a 10% solution of pyrrolidine in DMF at 90° C. for 1 minute in accordance with the method 2-4, followed by peptide chain elongation under conditions of the method 1-4 in the same manner as above. In this respect, Fmoc was removed from the fifth residue through reaction with a 10% solution of pyrrolidine in DMF at 90° C. for 1 minute in accordance with the method 2-4. Then, Alloc on the principal chain of the fifth residue was removed through reaction at room temperature for 60 minutes with a catalytic amount of tetrakis(triphenylphosphine) palladium (0) and 10 equivalents of phenylsilane in DCM with 2% HFIP. Subsequently, a peptide chain was elongated under conditions of the method 1-4 in the same manner as above. However, the reaction was performed twice at 90° C. for 10 minutes for the fourth residue. The reaction was performed once at 40° C. for 30 minutes for the first residue. In this respect, Fmoc was removed through reaction with a 10% solution of pyrrolidine in DMF at 90° C. for 1 minute in accordance with the method 2-4 or 2-5. However, the reaction was performed twice at room temperature for 1 minute for the fourth residue.

The chloroacetyl group was introduced by, after removal of the Fmoc group of an α-amino group by the method mentioned above, adding N-(chloroacetoxy) succinimide (approximately 5 equivalents) to the solid-phase resin carrying the Fmoc-protected peptide obtained in the preceding step in accordance with the method 3-6, and shaking the mixture at room temperature for 60 minutes in DMF. For the deprotection of a side chain and excision from the solid-phase resin, first, the resin obtained after the chloroacetyl group introduction step was washed with DMF and DCM, then washed with diethyl ether, and then dried under reduced pressure. Subsequently, the reactant cocktail (15 mL) of the method 4-1 was added to a reaction container containing the solid-phase resin in accordance with the method 4-1, and shaken at room temperature for 60 minutes. The reaction solution was collected by filtration from the frit. The solid-phase resin remaining in the reaction container was shaken again with the cocktail for excision, and a solution component was collected from the frit and mixed with the filtrate mentioned above. This filtrate was added to an excess of a diisopropyl ether/hexane (1/1) mixed solvent cooled to 0° C. so that cloudy precipitation occurred. This mixture was centrifuged, and the solution was decanted. The obtained solid was washed again with a small amount of diethyl ether and then dried under reduced pressure. The obtained solid was used in subsequent cyclization reaction. For the cyclization reaction of the peptide, the peptide was dissolved in a MeCN/H₂O (1/1) mixed solvent such that the final concentration of the peptide was 5 mM based on the number of moles of the solid-phase resin used. Then, 10 equivalents of triethylamine were added to the solution, and the mixture was stirred at room temperature for 6 hours. Then, the reaction solution was concentrated under reduced pressure.

The obtained crude product was purified under the following conditions (column: Phenomenex Kinetex EVO C18 5 μm, 30×150 mm; mobile phase: A=1.0% AcOH in H₂O, B=1.0% AcOH in MeCN; temperature: 40° C.; gradient (% B): 5-27% over 3 minutes, subsequently 27-32% over 8 minutes, and subsequently 32-60% over 1 minute; flow rate: 45 mL/min).

The purity of the compound of interest was calculated from an area ratio of an LC/MS (UV wavelength: 225 nm) chromatogram obtained under the following analysis condition and was consequently 93.8%.

Analysis condition Q-B: retention time=4.40 min

ESI-MS(+) found m/z=1411.06 (M+2H)²⁺

(calcd: 2819.20)

### Test Example 1: Test to Evaluate Intermolecular Interaction Between TNFR1 or TNER2 and Peptide by Surface Plasmon Resonance (SPR)

Each chemically synthesized peptide was evaluated for its intermolecular interaction with TNFR1 or TNFR2 by surface plasmon resonance (SPR) in accordance with a method given below. A specific testing method will be described below.

### (1) Measurement Method

CM5 sensor chip (Cytiva) was inserted to Biacore T200 (Cytiva), primed with a running buffer solution: HBS-EP+, pH 7.4 (Cytiva), and equilibrated at a flow rate of 30 μL/min.

Ligand immobilization operation was carried out at a flow rate of 10 μL/min. A 60 mM EDC solution (Cytiva) and a 650 mM NHS solution (Cytiva) were mixed in amounts each of 50 μL, then injected at a flow rate of 10 μL/min for 420 seconds, and reacted with a carboxy group on the CM5 sensor chip. A mixed solution of 100 nM TNFR1 or 100 nM TNFR2 and acetate, pH 5.0 (Cytiva) was reacted at a flow rate of 10 μL/min for 420 seconds to carry out amine coupling around 2000 R U. After immobilization, a 1.0 M aqueous ethanolamine solution (Cytiva) was reacted at a flow rate of 10 μL/min for 420 seconds for capping treatment.

A peptide solution prepared at 10 mM in a DMSO solution was diluted with a running buffer solution such that the peptide solution had a final peptide concentration of 10 μM. Then, the peptide solution was further diluted by any of four types of dilution methods (dilution series) of 1000-500-250-100-50 nM, 100-50-25-10-5 nM, 20-10-5-2-1 nM, and 10-5-2.5-1-0.5 nM using a running buffer solution containing 1% DMSO to prepare peptide solution groups containing 1% DMSO. Any of the peptide solution groups mentioned above was added at 25° C. at a flow rate of 30 μL/min for 120 seconds by the single cycle kinetics method, and dissociation was carried out for 600 seconds. The kinetics of the peptide for TNFR1 or TNER2 were obtained by SPR measurement.

Single cycle kinetics were used as a kinetics evaluation model, and curve fitting was performed using Biacore T200 Evaluation Software Version 3.0 (Cytiva). The obtained sensorgram was subjected to curve fitting by the least squares method. A KD value thereof was determined to evaluate the binding of the peptide to TNFR1 or TNFR2.

Each operation and condition in measurement can be appropriately changed or modified without largely influencing the measurement as long as those skilled in the art can understand the changes or the modifications.

### (2) Measurement Results

As shown in Table 10 below, the compounds according to Examples selectively bound to TNFR1 without binding to TNFR2, and thus exhibited excellent binding affinity and selectivity for TNFR1.

TABLE 10

| | TNFR1 | TNFR2 |
|---|---|---|
| Example | KD(nM) | KD(nM) |
| 1 | 0.26 | No signal |
| 2 | 0.04 | No signal |
| 3 | 0.04 | No signal |
| 4 | 0.05 | No signal |
| 5 | 0.05 | No signal |
| 6 | 0.06 | No signal |
| 7 | 0.04 | No signal |
| 8 | 0.04 | No signal |
| 9 | 0.09 | No signal |
| 10 | 0.07 | No signal |
| 11 | 0.05 | No signal |
| 12 | 0.11 | No signal |
| 13 | 0.03 | No signal |
| 14 | 0.01 | No signal |
| 15 | 0.02 | No signal |
| 16 | 0.02 | No signal |
| 17 | 0.06 | No signal |
| 18 | 0.03 | No signal |
| 19 | 0.02 | No signal |
| 20 | 0.03 | No signal |
| 21 | 0.11 | No signal |
| 22 | 0.07 | No signal |
| 23 | 0.04 | No signal |
| 24 | 0.03 | No signal |
| 25 | 0.08 | No signal |
| 26 | 0.01 | No signal |
| 27 | 0.03 | No signal |
| 28 | 0.07 | No signal |
| 30 | 0.08 | No signal |
| 31 | 0.09 | No signal |

### Test Example 2: Test to Inhibit TNFα-Stimulated IL-8 Production Using Human Fetal Lung Fibroblast (MRC-5)

### (1) Measurement Method

The MRC5 assay can evaluate the influence of a test substance on IL-8 production induced by TNFα stimulation. MRC5 cells (ATCC, Cat. CCL0-171) were inoculated at 5×10³ cells/100 μL/well to a 96-well plate and incubated overnight at 37° C. in a 5% CO₂ incubator. Then, a culture supernatant was aspirated, and 80 μL of a medium was newly added to each well. Then, 10 μL of a test substance was added to each well, followed by incubation at 37° C. for 1 hour. Next, 10 μL of TNFα (final concentration: 0.2 ng/ml, R&D systems, Inc., Cat. 210-TA-020) was added to each well, followed by further incubation for 18 hours. After culture, the plate was centrifuged, and 60 μL of a supernatant was collected from each well and used in the evaluation of an IL-8 concentration using Human IL-8/CXCL8 Quantikine ELISA Kit (R&D systems, Inc., Cat. PD8000C). The IL-8 concentration was evaluated in accordance with the manual of the ELISA Kit. Absorbance was measured at 450 nm and 570 nm with a microplate reader (Molecular Devices, LLC, VersaMAX), and the concentration of IL-8 contained in the culture supernatant was calculated from a calibration curve. The rate of suppression of IL-8 production without the addition of TNFα was defined as 100%, and the rate of suppression of IL-8 production without the addition of the test substance was defined as 08. IC₅₀ of the test substance was calculate using log (inhibitor) vs. response-Variable slope (four parameters) model of data analysis software GraphPad Prism10 (GraphPad Software).

Each operation and condition in measurement can be appropriately changed or modified without largely influencing the measurement as long as those skilled in the art can understand the changes or the modifications.

### (2) Measurement Results

As shown in Table 11 below, the compounds according to Examples exhibited excellent inhibition of IL-8 production. The calculated IC₅₀ of the test substance was rounded off to two decimal places and used as an evaluation value.

TABLE 11

| Example | IC50 (nM) |
|---|---|
| 1 | 0.19 |
| 2 | 0.14 |
| 3 | 0.18 |
| 4 | 0.18 |
| 5 | 0.14 |
| 6 | 0.23 |
| 7 | 0.34 |
| 8 | 0.10 |
| 9 | 0.17 |
| 10 | 0.08 |
| 11 | 0.12 |
| 12 | 0.16 |
| 13 | 0.19 |
| 14 | 0.17 |
| 15 | 0.15 |
| 16 | 0.10 |
| 17 | 0.16 |
| 18 | 0.07 |
| 19 | 0.13 |
| 20 | 0.12 |
| 21 | 0.14 |
| 22 | 0.17 |
| 23 | 0.18 |
| 24 | 0.17 |
| 25 | 0.16 |
| 26 | 0.18 |
| 27 | 0.07 |
| 28 | 0.19 |
| 29 | 1.29 |
| 30 | 0.25 |
| 31 | 0.20 |

### Test Example 3: Test to Suppress TNFα-Induced Cytokine Production in Blood Using Human TNFR1 Knock-In Mouse

A test substance can be evaluated for its suppressive effect on cytokine production induced by TNFα by a method given below. A specific testing method will be described below.

### (1) Measurement Method

The compound of Example 7 was subcutaneously administered at 0.1 mg/kg, 0.3 mg/kg, or 1.3 mg/kg to 11-week-old male hTNFR1 KI mice (Biomedcode Hellas SA) in which human TNFR1 was knocked in. Human TNFα was intravenously administered at a dose of 0.2 μg/100 μL/mouse (R&D Systems, Inc., Cat. 210-TA) 24 hours after subcutaneous administration. Blood was collected from the abdominal vena cava 4 hours after intravenous administration under isoflurane (Pfizer Japan Inc., Cat. 4987114133403) anesthesia using a syringe supplemented with an appropriate amount of heparin (Mochida Pharmaceutical Co., Ltd., Cat. 3334401A6158). The obtained blood was centrifuged to obtain plasma. The concentration of mouse IL-6 in the plasma was measured by use of the ECL method (MESO SCALE DISCOVERY, Cat. K152QXD-1).

A control group without administration of human TNFα (−) was defined as a 100% suppression control, and a vehicle administration (+) control group was defined as a 0% suppression control. The rate of suppression of mouse IL-6 production in each group relative to these controls was calculated using Excel2010 (Microsoft).

Each operation and condition in measurement can be appropriately changed or modified without largely influencing the measurement as long as those skilled in the art can understand the changes or the modifications.

### (1) Measurement Results

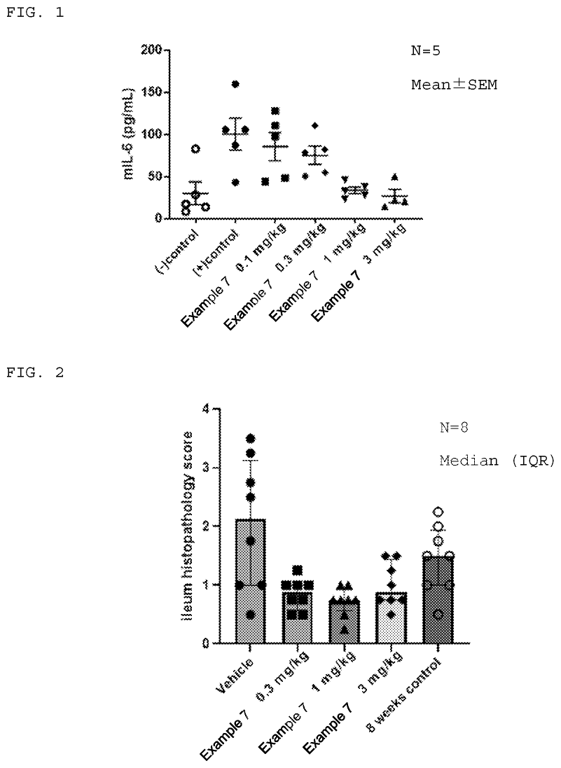

Results about the amount of mouse IL-6 produced are shown in FIG. 1. The ordinate depicts the amount of mouse IL-6 produced, and the abscissa depicts a group name. “n” represents the number of mice used.

The compound according to Example 7 subcutaneously administered at 0.1 mg/kg, 0.3 mg/kg, 1 mg/kg, and 3 mg/kg had 218, 31%, 95%, and 105% effects, respectively, of suppressing mouse IL-6 production.

Thus, the compound according to an embodiment (Example 7) exhibited an excellent cytokine suppressive effect.

### Test Example 4: Evaluation of Drug Efficacy of TNF-ΔARE/Human TNFR1 Knock-In Mouse on Enteritis

A test substance can be evaluated for its drug efficacy on enteritis by a method given below. A specific testing method will be described below.

### (1) Measurement Method

The compound of Example 7 was subcutaneously administered once a day at 0.3 mg/kg or 1.3 mg/kg by repetitive dosing from 8 weeks to 16 weeks of age to TNF-ΔARE/human TNFR1 KI mice (Biomedcode Hellas SA) obtained by the knock-in of human TNFR1 in TNF-ΔARE mice which spontaneously develop ileitis by the high expression of TNF. The ileum was excised from the mice at the start of administration for an 8-week control group and after 8-week administration for the administration groups, and then fixed in a 4% formaldehyde solution. The fixed ileum was embedded in paraffin, and sections of three different sites on the sagittal section were prepared and stained with HE to prepare pathological specimens. Three pathological specimens per individual were used and evaluated by pathological scoring in accordance with pathological criteria for scoring ileum inflammation shown in Table 12. Each operation and condition in measurement can be appropriately changed or modified without largely influencing the measurement as long as those skilled in the art can understand the changes or the modifications.

TABLE 12
HISTOPATHOLOGICAL CRITERIA FOR
SCORING ILEUM INFLAMMATION

| SCORE¹ | CRITERIA |
|---|---|
| 0 | No detectable pathology |
| 1 | Isolated inflammatory cells in stroma |
| 2 | Inflammatory cells in groups with focal infiltration to |
| | mucosa and submucosa layer |
| 3 | Inflammatory cells in groups with extensive mucosal and |
| | submucosal inflammation |
| 4 | Transmural inflammation |

¹Intermediate histopathology scores (e.g. 0.25, 0.5, 0.75) refer to the extent of the histopathological features. Generally, when a specific score applies to 25% or less of the total ileum length (about 6 cm), then the total score is accordingly reduced. Intermediate marks are also added when a specific score applies to more than 50% of the total ileum length. For example, when the histopathological features of a score 2 ileum are focal, then the total score will be less than 2, for example 1.75, but it will be 2.5 or 2.75 when these features apply to the whole specimen.

### (2) Measurement Results

Results of evaluating enteritis (ileum inflammation) by pathological scoring are shown in FIG. 2. The ordinate depicts a pathological score of enteritis, and the abscissa depicts a group name. “n” represents the number of mice used.

The compound of Example 7 subcutaneously administered once a day at 0.3 mg/kg or 1, 3 mg/kg by repetitive dosing had a lower pathological score of enteritis than that of the vehicle administration group.

Thus, the compound according to an embodiment (Example 7) exhibited excellent drug efficacy on enteritis.

### Test Example 5: Comparison with Compound of Conventional Technique

WO2024/163691 describes TNFR1-inhibitory peptides having a structure partially similar to that of the compound of the formula (1).

Pages 236 to 240 of WO2024/163691 describe measurement methods of biological assay. Pages 266 to 273 of WO2024/163691 describe measurement results thereof.

Table 13 below shows peptides (C-1, C-2, and C-3) structurally most analogous to the compound of the formula (1) among the peptides of Examples described in WO2024/163691.

TABLE 13

| Example | Example No. of | |
|---|---|---|
| No. | WO2024/16369 | Sequence and structure of peptide |
| C-1 | 265 | ClAc-homoF-Y-S-1inda-MeA-N-4Py-W-aMeS-F4COO-Y- MeG-W-C-NH2 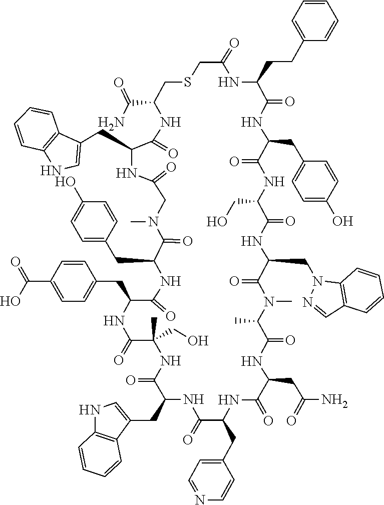 [SMILES: C[C@H]1C(=O)N[C@@H](CC(N)=O)C(=O)N[C@@H](Cc2ccncc2)C(=O)N[C@@H](Cc2c[nH]c3ccccc23)C(=O)N[C@@](C)(CO)C(=O)N[C@@H](Cc2ccc(C(=O)O)cc2)C(=O)N[C@@H](Cc2ccc(O)cc2)C(=O)N(C)CC(=O)N[C@@H](Cc2c[nH]c3ccccc23)C(=O)N[C@H](C(N)=O)CSCC(=O)N[C@@H](CCc2ccccc2)C(=O)N[C@@H](Cc2ccc(O)cc2)C(=O)N[C@@H](CO)C(=O)N[C@@H](Cn2ncc3ccccc32)C(=O)N1C] |
| C-2 | 263 | ClAc-homoF-Y-S-W7F-MeA-N-4Py-W-aMeS-F4COO-Y- MeG-W-C-NH2 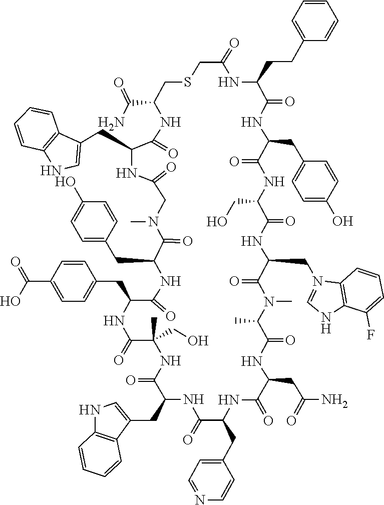 |
| C-3 | 233 | ClAc-homoF-4Py-Dap-W-dP-N-4Py-2Nal-aMeS-F4COO- 3Py-MeG-W-C-NH2 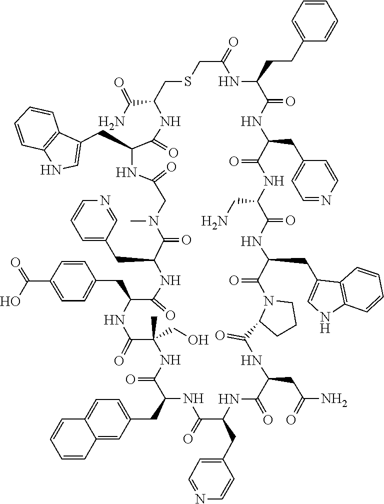 [SMILES: CN1CC(=O)N[C@@H](Cc2c[nH]c3ccccc23)C(=O)N[C@H](C(N)=O)CSCC(=O)N[C@@H](CCc2ccccc2)C(=O)N[C@@H](Cc2ccncc2)C(=O)N[C@@H](CN)C(=O)N[C@@H](Cc2c[nH]c3ccccc23)C(=O)N2CCC[C@@H]2C(=O)N[C@@H](CC(N)=O)C(=O)N[C@@H](Cc2ccncc2)C(=O)N[C@@H](Cc2ccc3ccccc3c2)C(=O)N[C@@](C)(CO)C(=O)N[C@@H](Cc2ccc(C(=O)O)cc2)C(=O)N[C@@H](Cc2cccnc2)C1=O] |

These compounds C1 to C3 were evaluated for their ability to inhibit IL-8 production in human fetal lung fibroblasts (MRC-5) by the method described in <Test Example 2>. The results about each compound are shown in Table 14. Table 14 also describes the result of evaluating the compound of Example 7 of the present invention to be compared.

TABLE 14

| | Example Number | MRC5 |
|---|---|---|
| Example | in WO2024163691 | IC₅₀ (nM) |
| C-1 | 265 | 12.97 |
| C-2 | 263 | 5.95 |
| C-3 | 233 | 8.61 |
| 7 | — | 0.34 |

The compound of Example 7 was superior in binding affinity for TNFR1 and ability to inhibit IL-8 production in human fetal lung fibroblasts (MRC-5) to all the compounds C-1 to C-3.

The following measurement of Test Examples 6 and 7 may be performed in order to evaluate the action and activity of the compound of the formula (1).

### Test Example 6: Ex Vivo Cytokine Assay Using Peripheral Blood of Cynomolgus Monkey Given Test Substance

A test substance can be evaluated for its suppressive effect on cytokine production caused by TNFα stimulation in ex vivo assay using peripheral blood of cynomolgus monkeys, for example, by a method given below.

### (1) Administration of Test Substance to Cynomolgus Monkey, Blood Collection Method, and Cytokine Measurement Method

A test substance is subcutaneously administered at 0.1 mg/kg, 0.3 mg/kg, or 1.3 mg/kg to the back. Blood is collected using a syringe supplemented with heparin sodium 24 hours after subcutaneous administration. The obtained cynomolgus monkey peripheral blood is added to each well of a 96-well plate, and TNFα or a blank vehicle is added to the peripheral blood and incubated. After culture, the plate is centrifuged, and the amount of IL-8 in the obtained supernatant is measured with ELISA Kit.

Each operation and condition can be appropriately changed or modified as long as those skilled in the art can understand the changes or the modifications.

### Test Example 7: Test to Inhibit TNFα-Stimulated IL-8 Production Using Human Peripheral Blood

A test substance can be evaluated for its suppressive effect on cytokine production caused by TNFα stimulation in ex vivo assay using human peripheral blood, for example, by a method given below.

### (1) Measurement Method

Fresh human peripheral blood is obtained by blood collection with added heparin. The human peripheral blood is added to each well of a 96-well plate, and a test substance is added thereto and incubated at 37° C. Subsequently, TNFα or a blank vehicle is added thereto and further incubated. After culture, the plate is centrifuged, and a supernatant is collected. An IL-8 concentration is measured with ELISA Kit.

Each operation and condition in measurement can be appropriately changed or modified as long as those skilled in the art can understand the changes or the modifications.

1. A compound represented by Formula (1) or a salt thereof: 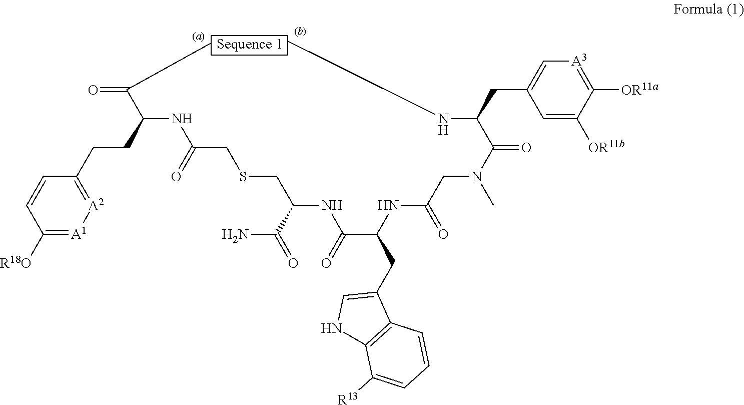
wherein
the sequence 1 is an amino acid sequence represented by formula (1-1): 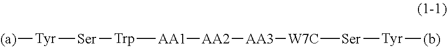
A¹ and A² are each independently CH or N;
R^{1a} is C₁₋₃ alkyl;
A³ is CH or N;
R^{11a} and R^{11b} are each independently C₁₋₃ alkyl;
R¹³ is 5- or 6-membered heteroaryl, and R¹³ is optionally substituted with one or two identical or different substituents selected from a G^{Ar} group;
the G^{Ar} group is the group consisting of —F, —Cl, cyano, C₁₋₃ alkyl, halogeno-C₁₋₃ alkyl, C₁₋₃ alkoxy-C₂₋₃ alkyl, —C(O)OH, and —C(O)NH₂;
AA1 is an amino acid structure represented by formula (2-1): 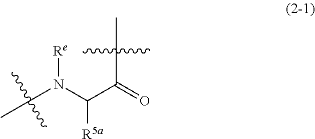
R^{e} is —H or C₁₋₃ alkyl, and C₁₋₃ alkyl of R^{e} is optionally substituted with —C(O)OH;
R^{5a} is —H, C₁₋₄ alkyl, halogeno-C₁₋₃ alkyl, or C₁₋₃ alkoxy-C₁₋₃ alkyl, and R^{5a} is optionally substituted with a substituent selected from a G¹ group;
the G¹ group is the group consisting of hydroxy, —NHR^{5b}, —C(O)OH, and —C(O)NH₂;
R^{5b} is a residue represented by formula (2-1-1); 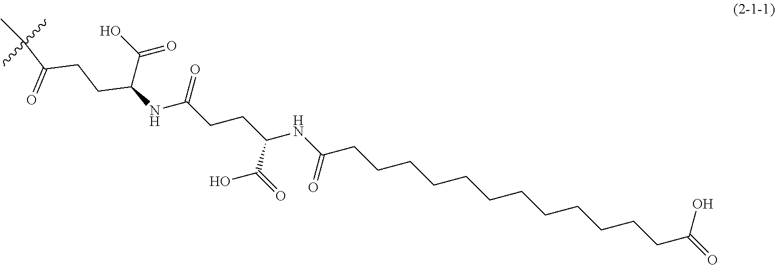 [SMILES: *C(=O)CC[C@H](NC(=O)CC[C@H](NC(=O)CCCCCCCCCCCCC(=O)O)C(=O)O)C(=O)O]
AA2 is an amino acid structure represented by formula (2-2): 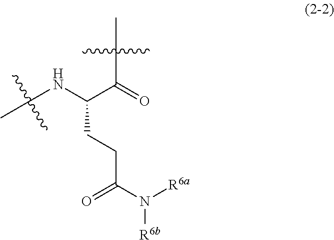
or an amino acid structure represented by formula (2-3): 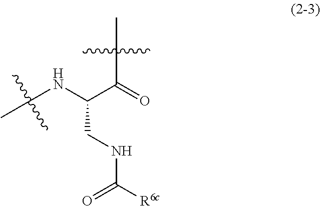
R^{6a} and R^{6b} are each independently —H, C₁₋₄ alkyl, halogeno-C₁₋₄ alkyl, hydroxy-C₁₋₄ alkyl, C₁₋₃ alkoxy-C₁₋₄ alkyl, or a 3- to 6-membered saturated ring group, and each of R^{6a} and R^{6b} is optionally substituted with a substituent selected from a G² group;
the G² group is the group consisting of —NH₂, 5- or 6-membered heteroaryl, and a 4- to 6-membered saturated ring group, and the 4- to 6-membered saturated ring group optionally has an N atom; or
R^{6a} and R^{6b} optionally form a 4- to 6-membered saturated ring together, and the 4- to 6-membered saturated ring is optionally substituted with C₁₋₃ alkyl-NH₂;
R^{6c} is C₁₋₃ alkyl;
AA3 is an amino acid structure represented by formula (2-4): 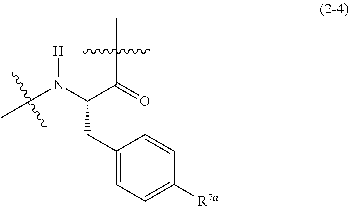
and
R^{7a} is —H, hydroxy, or —C(O)OH.

2. (canceled)

3. The compound or the salt thereof according to claim 1, wherein
A¹ is CH, A² is N, and
R^{1a} is methyl.

4. (canceled)

5. The compound or the salt thereof according to claim 1, wherein
A³ is N, and
both of R^{11a} and R^{11b} are methyl.

6. The compound or the salt thereof according to claim 1, wherein
A³ is CH, and
both of R^{11a} and R^{11b} are methyl.

7.-9. (canceled)

10. The compound or the salt thereof according to claim 1, wherein R¹³ is pyrazolyl, pyridyl, pyridazinyl, or pyrimidinyl.

11. (canceled)

12. The compound or the salt thereof according to claim 1, wherein the G^{Ar} group is the group consisting of —F, methyl, —C(O)OH, and —C(O)NH₂.

13. (canceled)

14. The compound or the salt thereof according to claim 1, wherein R¹³ has the following structure:

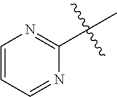 [SMILES: *c1ncccn1]

15. (canceled)

16. The compound or the salt thereof according to claim 1, wherein R¹³ is pyridyl substituted with one —C(O)OH.

17. The compound or the salt thereof according to claim 1, wherein R¹³ is pyridyl substituted with one —C(O)NH₂.

18. The compound or the salt thereof according to claim 1, wherein R¹³ is pyridazinyl.

19. The compound or the salt thereof according to claim 1, wherein R¹³ is pyrimidinyl substituted with one —F.

20. (canceled)

21. The compound or the salt thereof according to claim 1, wherein
R^{5a} is —H or C₁₋₃ alkyl, and
the G¹ group is —C(O)OH.

22. The compound or the salt thereof according to claim 1, wherein
R^{e} is methyl,
R^{5a} is methyl or ethyl, and
the G¹ group is —C(O)OH.

23. The compound or the salt thereof according to claim 1, wherein
both of R^{e} and R^{5a} are methyl.

24. The compound or the salt thereof according to claim 1, wherein
R^{e} is ethyl substituted with —C(O)OH, and
R^{5a} is —H.

25. The compound or the salt thereof according to claim 1, wherein
R^{e} is methyl,
R^{5a} is butyl substituted with —NHR^{5b}, and
R^{5b} is a residue represented by the general formula (2-1-1).

26. (canceled)

27. The compound or the salt thereof according to claim 1, wherein
AA2 is an amino acid represented by the formula (2-2), and
each of R^{6a} and R^{6b} is —H.

28. (canceled)

29. (canceled)

30. The compound or the salt thereof according to claim 1, wherein
AA2 is an amino acid represented by the formula (2-2), and
one of R^{6a} and R^{6b} is —H, and the other is ethyl substituted with nitrogen-containing 6-membered heteroaryl.

31.-33. (canceled)

34. The compound or the salt thereof according to claim 1, wherein
AA2 is an amino acid represented by the formula (2-3); and
R^{6c} is methyl.

35. The compound or the salt thereof according to claim 1, wherein
R^{7a} is hydroxy.

36. The compound or the salt thereof according to claim 1, wherein
R^{7a} is —C(O)OH.

37. (canceled)

38. A method for inhibiting TNFR1, comprising:
administering the compound or the salt thereof according to claim 1 to a subject in need thereof.

39. A pharmaceutical composition, comprising:
the compound or the salt thereof according to claim 1 as an active ingredient, and
an additional pharmaceutically acceptable component.

40. A method for treating a disease related to inhibition of TNER1, comprising:
administering the pharmaceutical composition according to claim 39 to a subject in need thereof.

41. The method according to claim 40, wherein the disease is selected from the group consisting of ulcerative colitis, Crohn's disease, rheumatoid arthritis, psoriasis, ankylosing spondylitis, and hidradenitis suppurative.

42. The method according to claim 40, wherein the disease is Crohn's disease.

43. (canceled)

44. The method according to claim 40, wherein the pharmaceutical composition is subcutaneously administered.

45. A compound represented by Formula (3) or a salt thereof:

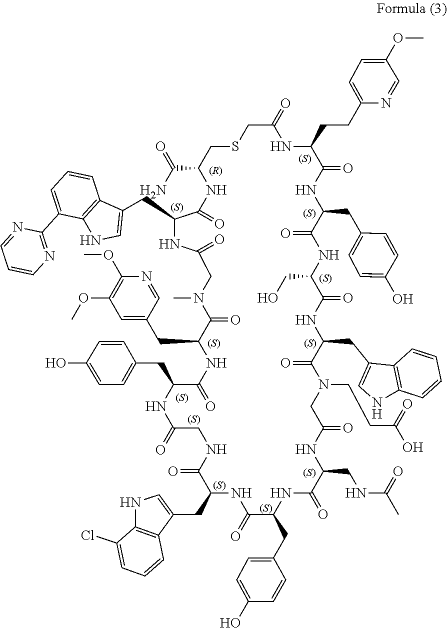

46. A method for producing a compound represented by Formula (1) or a salt thereof: 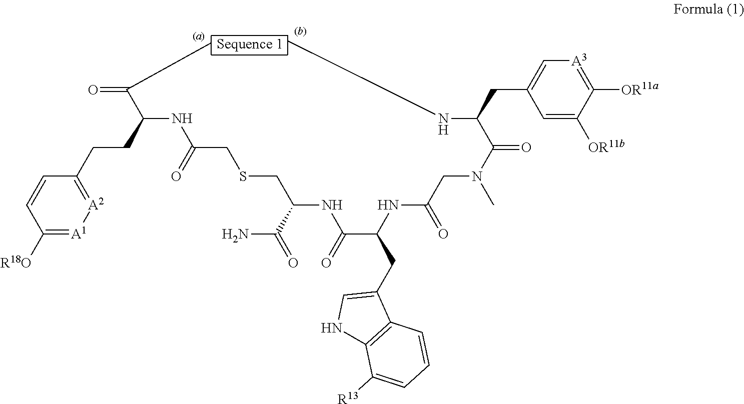
wherein
the sequence 1 is an amino acid sequence represented by formula (1-1): 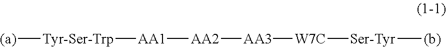
A¹ and A² are each independently CH or N;
R^{1a} is C₁₋₃ alkyl;
A³ is CH or N;
R^{11a} and R^{11b} are each independently C₁₋₃ alkyl;
R¹³ is 5- or 6-membered heteroaryl, and R¹³ is optionally substituted with one or two identical or different substituents selected from a G^{Ar} group;
the G^{Ar} group is the group consisting of —F, —Cl, cyano, C₁₋₃ alkyl, halogeno-C₁₋₃ alkyl, C₁₋₃ alkoxy-C₂₋₃ alkyl, —C(O)OH, and —C(O)NH₂;
AA1 is an amino acid structure represented by formula (2-1): 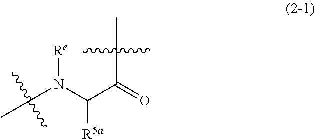
R^{e} is —H or C₁₋₃ alkyl, and C₁₋₃ alkyl of R^{e} is optionally substituted with —C(O)OH;
R^{5a} is —H, C₁₋₄ alkyl, halogeno-C₁₋₃ alkyl, or C₁₋₃ alkoxy-C₁₋₃ alkyl, and R^{5a} is optionally substituted with a substituent selected from a G¹ group;
the G¹ group is the group consisting of hydroxy, —NHR^{5b}, —C(O)OH, and —C(O)NH₂;
R^{5b} is a residue represented by formula (2-1-1); 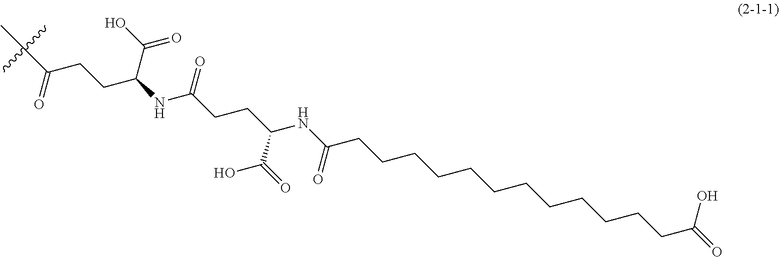 [SMILES: *C(=O)CC[C@H](NC(=O)CC[C@H](NC(=O)CCCCCCCCCCCCC(=O)O)C(=O)O)C(=O)O]
AA2 is an amino acid structure represented by formula (2-2): 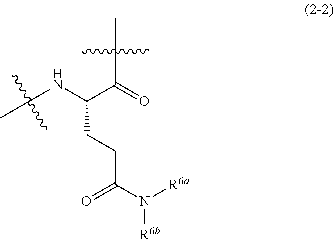
or an amino acid structure represented by formula (2-3): 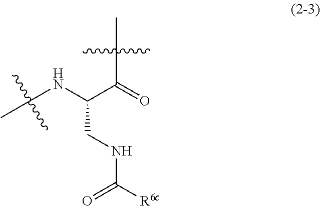
R^{6a} and R^{6b} are each independently —H, C₁₋₄ alkyl, halogeno-C₁₋₄ alkyl, hydroxy-C₁₋₄ alkyl, C₁₋₃ alkoxy-C₁₋₄ alkyl, or a 3- to 6-membered saturated ring group, and each of R^{6a} and R^{6b} is optionally substituted with a substituent selected from a G² group;
the G² group is the group consisting of —NH₂, 5- or 6-membered heteroaryl, and a 4- to 6-membered saturated ring group, and the 4- to 6-membered saturated ring group optionally has an N atom; or
R^{6a} and R^{6b} optionally form a 4- to 6-membered saturated ring together, and the 4- to 6-membered saturated ring is optionally substituted with C₁₋₃ alkyl-NH₂;
R^{6c} is C₁₋₃ alkyl;
AA3 is an amino acid structure represented by formula (2-4): 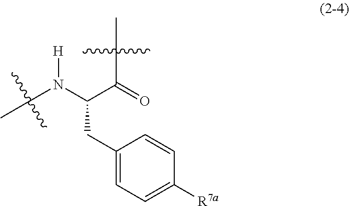
and
R^{7a} is —H, hydroxy, or —C(O)OH,
the method comprising:
providing 14 amino acids constituting the Formula (1), the amino acids being N-terminally protected with a 9-fluorenylmethyloxycarbonyl (Fmoc) group;
repeating an operation of sequentially coupling the amino acids and removing the Fmoc group from the coupled amino acids to form a peptide; and
cyclizing the peptide to obtain the compound represented by the Formula (1) or the salt thereof.

47. A cyclic peptide selected from the group consisting of:
(1) ClAc—H2PyOMe-Y—S—W-MeA-Qpyrli3RMe-F4COO—W7C—S—Y—F34dOMe-MeG-W72Py-C—NH2;
(2) ClAc—H2PyOMe-Y—S—W-MeA-QaEt-Y—W7C—S—Y-3Py56dOMe-MeG-W72pyrm-C—NH2;
(3) ClAc—H2PyOMe-Y—S—W-MeA-Qpyrli3RMe-F4COO—W7C—S—Y-3Py56dOMe-MeG-W72pyrm-C—NH2;
(4) ClAc—H2PyOMe-Y—S—W-MeA-QaPr—F4COO—W7C—S—Y-3Py56dOMe-MeG-W72pyrm-C—NH2;
(5) ClAc—H2PyOMe-Y—S—W-MeA-DapAc—Y—W7C—S—Y-3Py56dOMe-MeG-W72pyrm-C—NH2;
(6) ClAc—H2PyOMe-Y—S—W-MeA-Qpyrli3RMe-F4COO—W7C—S—Y-3Py56dOMe-MeG-W72Pyrm5F—C—NH2;
(7) ClAc—H2PyOMe-Y—S—W—CeG-DapAc—Y—W7C—S—Y-3Py56dOMe-MeG-W72pyrm-C—NH2;
(8) ClAc—H2PyOMe-Y—S—W-MeA-Q-Y—W7C—S—Y-3Py56dOMe-MeG-W72pyrm-C—NH2;
(9) ClAc—H2PyOMe-Y—S—W-MeA-Q-F4COO—W7C—S—Y-3Py56dOMe-MeG-W72pyrm-C—NH2;
(10) ClAc—H2PyOMe-Y—S—W—CeG-Q-Y—W7C—S—Y-3Py56dOMe-MeG-W72pyrm-C—NH2;
(11) ClAc—H2PyOMe-Y—S—W—CeG-Qpyrli3RMe-Y—W7C—S—Y-3Py56dOMe-MeG-W72pyrm-C—NH2;
(12) ClAc—H2PyOMe-Y—S—W-MeA-Q4Pye-F4COO—W7C—S—Y-3Py56dOMe-MeG-W72pyrm-C—NH2;
(13) ClAc—H2PyOMe-Y—S—W-MeD-DapAc—Y—W7C—S—Y-3Py56dOMe-MeG-W72pyrm-C—NH2;
(14) ClAc—H2PyOMe-Y—S—W-MeA-Qpyrli3RMe-F4COO—W7C—S—Y-3Py56dOMe-MeG-W72Py-C—NH2;
(15) ClAc—H2PyOMe-Y—S—W-MeA-QaMe3Az-F4COO—W7C—S—Y-3Py56dOMe-MeG-W72Py-C—NH2;
(16) ClAc—H2PyOMe-Y—S—W-MeA-QaMe3Az-Y—W7C—S—Y-3Py56dOMe-MeG-W73Pyrd-C—NH2;
(17) ClAc—H2PyOMe-Y—S—W-MeA-DapAc-F4COO—W7C—S—Y-3Py56dOMe-MeG-W73Pyrd-C—NH2;
(18) ClAc—H2PyOMe-Y—S—W-MeA-DapAc—Y—W7C—S—Y-3Py56dOMe-MeG-W73Pyrd-C—NH2;
(19) ClAc—H2PyOMe-Y—S—W-MeA-QaMe3Az-F4COO—W7C—S—Y—F34dOMe-MeG-W72Py5COO—C—NH2;
(20) ClAc—H2PyOMe-Y—S—W-MeA-QaMe3Az-F4COO—W7C—S—Y—F34dOMe-MeG-W72pyrm-C—NH2;
(21) ClAc—H2PyOMe-Y—S—W—CeG-QaMe3Az-F4COO—W7C—S—Y—F34dOMe-MeG-W72pyrm-C—NH2;
(22) ClAc—H3Pyrd60Me-Y—S—W-MeA-QaMe3Az-F4COO—W7C—S—Y-3Py56dOMe-MeG-W72pyrm-C—NH2;
(23) ClAc—H2PyOMe-Y—S—W-MeE-QaPr—Y—W7C—S—Y-3Py56dOMe-MeG-W72pyrm-C—NH2;
(24) ClAc—H2PyOMe-Y—S—W-MeA-QaPr—F4COO—W7C—S—Y-3Py56dOMe-MeG-W73Pyz1Me-C—NH2;
(25) ClAc—H2PyOMe-Y—S—W-MeA-DapAc—Y—W7C—S—Y-3Py56dOMe-MeG-W72Py5COO—C—NH2;
(26) ClAc—H2PyOMe-Y—S—W-MeA-Qpyrli3RMe-Y—W7C—S—Y-3Py56dOMe-MeG-W72Py5CON—C—NH2;
(27) ClAc—H2PyOMe-Y—S—W-MeA-QaMe3Az-F4COO—W7C—S—Y—F34dOMe-MeG-W72Py5CON—C—NH2;
(28) ClAc—H2PyOMe-Y—S—W-MeA-QaMe3Az-F4COO—W7C—S—Y-3Py56dOMe-MeG-W73Pyrd-C—NH2;
(29) ClAc—H2PyOMe-Y—S—W-MeK (cC12COO-isoE-isoE-)-QaPr—F4COO—W7C—S—Y-3Py56dOMe-MeG-W72pyrm-C—NH2;
(30) ClAc—H2PyOMe-Y—S—W—CeG-DapAc-F4COO—W7C—S—Y-3Py56dOMe-MeG-W72pyrm-C—NH2; and
(31) ClAc—H2PyOMe-Y—S—W—CeG-Q-F4COO—W7C—S—Y-3Py56dOMe-MeG-W72pyrm-C—NH2,
or a salt thereof.
